(19) United States
(12) Patent Application Publication
(10) Pub. No.: US 2024/0335470 A1
(43) Pub. Date: Oct. 10, 2024

(54) CAR T CELLS FOR TREATING CD19+, CD20+ OR CD22+ TUMORS OR B-CELL DERIVED AUTO-IMMUNE DISEASES

(71) Applicant: OSPEDALE PEDIATRICO BAMBINO GESU', Roma (IT)

(72) Inventors: Concetta QUINTARELLI, Roma (IT); Biagio DE ANGELIS, Roma (IT); Franco LOCATELLI, Roma (IT)

(21) Appl. No.: 18/266,493

(22) PCT Filed: Dec. 10, 2021
(86) PCT No.: PCT/IT2021/050402

§ 371 Date: Jun. 9, 2023

(30) Foreign Application Priority Data
Dec. 10, 2020 (IT) 102020000030266

(51) Int. Cl. A61K 35/17; A61K 39/00; A61P 35/02; C07K 16/28; C12N 5/0783; C12N 15/85

(52) U.S. Cl. CPC A61K 35/17; A61K 39/4611; A61K 39/4613; A61K 39/4631; A61K 39/464412; A61K 39/464413; A61K 39/464424; A61P 35/02; C07K 16/2803; C07K 16/2887; C12N 5/0636; C12N 5/0646; C12N 15/85; A61K 2239/13; A61K 2239/17; A61K 2239/21; A61K 2239/25; A61K 2239/48

(57) ABSTRACT

CAR T cells for treating CD19+, CD20+ OR CD22+ tumors, including leukemia and lymphoid malignances, provide increased safety in the therapy of the tumors and prevent epitope masking in CAR+ B-cell leukemia blasts. The CAR T cells decrease the potential risk of CD19−/CAR+, CD20−/CAR+ or CD22−/CAR+ leukemic relapse. In addition, the CAR T cells provide increased safety in the treatment of autoimmune diseases caused by B cells producing auto-antibodies.

### PRIORITY AND CROSS-REFERENCE TO RELATED APPLICATIONS

This application is the U.S. National Stage Application under 35 U.S.C. § 371 of International Application No. PCT/IT2021/050402, filed Dec. 10, 2021, designating the U.S. and published in the English language as WO 2022/123613 A1 on Jun. 16, 2022, which claims the benefit of Italian Application No. IT 102020000030266, filed on Dec. 10, 2020. Each of these applications is incorporated herein by reference in its entirety.

### REFERENCE TO ELECTRONIC SEQUENCE LISTING

The present application is being filed along with an Electronic Sequence Listing. The Electronic Sequence Listing is provided as a file entitled BARZ038003APCSEQLISTING.txt, which is 85,282 bytes in size, which was created on Jun. 8, 2023, and which was last modified on Jun. 9, 2023, which is replaced by a Replacement Electronic Sequence Listing submitted herewith as a file entitled BARZ038003APCREPLACEMENTSEQLIST.txt, which is 85,321 bytes in size, and which was created on Jan. 2, 2024. The information in the Electronic Sequence Listing is incorporated herein by reference in its entirety.

The present invention concerns CAR T cells for treating CD19+, CD20+ OR CD22+ tumors or autoimmune diseases caused by B cells producing auto-antibodies. In particular, the present invention concerns powerful CAR T cells for treating CD19+, CD20+ OR CD22+ tumors, such as leukemia and lymphoid malignances, which provide increased safety in the therapy of said tumors and prevent epitope masking in CAR+ B-cell leukemia blasts, said CAR T cells being able to decrease the potential risk of CD19−/CAR+, CD20−/CAR+ or CD22−/CAR+ leukemic relapse. In addition, CAR T cells according to the present invention provides increased safety also in the treatment of autoimmune diseases caused by B cells producing auto-antibodies.

Patient-derived T cells genetically modified to express chimeric antigen receptors (CARs) specific for CD19 represent a novel, effective option for patients with relapsed/refractory B-cell precursor acute lymphoblastic leukemia (Bcp-ALL)^{1,2}. Indeed, two recent shortcut FDA approvals (the Novartis' CD19-targeting CAR T cell product Kymriah™ and the Kite's product Yescarta™, based on a lentiviral and retroviral platform, respectively) have highlighted the rapid pace of progress made in this field. In view of the exciting results reported in patients with CD19+ malignancies given CAR T-cells^{3,4}, it is expected that a continuously growing number of patients will be considered for this treatment and, thus, will be exposed to gene-modified products. Since the techniques of gene manipulation are relatively new, some of the delayed side effects associated with CAR T-cell therapy are still unpredictable and medical researchers, institutions, and regulatory agencies are working to ensure that gene therapy is as safe as possible.

In the case of B-ALL, CAR T-cell therapy causes rapid and sustained clinical response, but a barrier to widespread is represented by life-threatening reactions such as relapse, cytokine release syndrome (CRS), and on-target off-tumor effect.

Relapse is a tough step to overcome for CAR T-cell anti-leukemia therapy, despite the high initial complete remission rates. Early relapses, CD19 positive (or CD20/CD22 positive for which early relapses are also expected), are related to the short in vivo persistence of CAR T-cells, or its inhibition induced by microenvironment. Over time, up to 30% of patients relapses are characterized by CD19-negative B-ALL progression, in which the loss of antigen occurs, due to an alternative exon splicing, or gene deletion. The mutation renders the tumor cells invisible again to the armored CAR-T⁵.

CRS is an immune-mediated disorder characterized by the activation of large number of T cells and an excessive secretion of inflammatory cytokines leading to visceral or vascular endothelial injury, heart failure and respiratory distress among other complications that can be fatal⁶.

On-target off-tumor is due to a reactivity of CAR-T cells against normal tissues, expressing the tumor associated antigens. So, the targeted antigens should be as specific as possible to reduce the off-tumor targeting.

In the contest of CAR.CD19 T cell treatment, the off-tumor action of T cells in patients is associated with long-term B cell aplasia⁷.

As a safety point, modification of the CAR construct to selectively eliminate adoptively transferred T cells is becoming a critical step for the successful clinical translation of this approach.

The inclusion of a suicide gene provides an additional safety measures in the event of serious toxicity.

Several switches have been developed, based on genetic integration of a transgenic enzyme to activate a cytotoxic pro-drug (HSV-TK), or surface molecules (CD20, EGFR) mediating an antibody-dependent depletion mechanisms of genetically modified T cell^{8,9}.

The clinical use of the suicide gene inducible caspace 9 (iC9) has also been reported in patients who received a haplo-identical hematopoietic stem cell transplant, in which donors' lymphocytes were modified to improve GVHD or CRS control. The infusion of AP1903 drug initiates cell apoptosis via activation of iC9 dimerization^{10,11}

In the field of CAR T cells, the presence of leukemic clonotypes in patient-derived Drug Products (DPs) obtained through a gene manipulation based on a lentiviral vector encoding for a second-generation CAR.CD19 was reported. In particular, two Bcp-ALL patients who relapsed with CD19-negative, CAR.CD19-expressing B leukemia have been reported. This observation being interpretable as inadvertent leukemic B cell transduction with second-generation CAR.CD19 lentivirus during CAR T-cell manufacturing. This leukemic clone resulted to be resistant to CAR.CD19 T cell killing in a xenograft model12. Basically, in the clinical practice of patients receiving standard CAR.CD19 T cell infusion, it was discovered the cases of patients that relapsed with a leukemia expressing the CAR.CD19. In the same CAR+ leukemia, the target antigen CD19 was not anymore detectable, for a masking effect of the CAR itself on the B cell leukemia.

Next-generation immunoglobulin heavy chain sequencing (NGIS) analysis of 17 additional infusion products also identified the leukemic clonotypes in six additional products (35%).

In vitro and in vivo experiments proved that these CAR+leukemia cell clones were not killed by CAR.CD19 T-cells¹². Therefore, CAR T-cell approaches revealed a potential risk of CD19−/CAR+ leukemic relapse. A study shows that CAR+ leukemia cell clones can be controlled by an anti-CAR.CD19 idiotype CAR¹³. According to this approach, both CAR-T and anti-CAR cells should be generated for each patient with a consequent increasing of costs.

Current treatments for autoimmune disease mainly depend on the use of nonsteroidal anti-inflammatory drugs, antimalarial drugs, glucocorticoids, and immunosuppression for severe symptoms with organ dysfunction. As B cells play a central role in the pathogenesis of these diseases, many B-cell-directed immunotherapies have been recently developed. However, these therapies have shown only modest success in the subgroup of SLE patients with serologically active disease. The anti-BAFF (B-cell-activating factor) agent belimumab, which was the first targeted biological treatment for SLE, has obtained approval from the Food and Drug Administration and the European Medicines Agency but can only partially deplete naive B cells. In contrast, two other BAFF-blocking agents, tabalumab and blisibimod, showed negative results in clinical trials for SLE treatment. Rituximab, an antibody against CD20, can deplete B cells more efficiently, but response rates in SLE patients vary widely between studies. Disease relapse after treatment with rituximab remains a problem. B cell markers, as CD19, CD20 and CD22 expression is maintained at a high level throughout all stages of B-cell differentiation. Thus, they are considered a good target to achieve more efficient and long-lasting therapeutic responses in these patients. For this reason, the transfer of autologous T cells expressing anti-CD19 chimeric antigen receptors has been used to treat three patients with SLE. (DOI: 10.1056/NEJMc2107725).

As the number of patients that can benefit from CAR T infusion is increasing, it is becoming mandatory to increase the level of safety of the gene therapy approach.

In the light of the above, it is therefore apparent the need to provide for further CD19, CD20 and CD22-targeting CAR T cells, which are able to overcome the disadvantages of the known CAR T cells.

In this contest, the aim of the present invention is to increase the safety of CD19, CD20 and CD22-targeting CAR T cell gene therapy.

This aim, besides being relevant in cancer therapy, is also relevant when CAR T cells are used in patients with autoimmune diseases caused by B cells generating auto-antibodies, including but not only limited to systemic lupus erythematosus (SLE), Systemic sclerosis (SSc), ANCA-Associated Vasculitis (AAV), Dermatomyositis (DM).

According to the present invention, it has been now found that a CAR comprising a short linker is a molecule with an increased level of security respect to standard conformation of CAR, since it is able to recognize and kill unwanted CAR+ tumor B cells.

According to the present invention, it is now provided a new CAR CD19, CD20 or CD22 design able to provide protection in the unlikely case of CAR+ leukemic B cell generation, even when CAR T-cell production was started from patient-derived material rich in leukemic cells (peripheral blood with more than 45% of leukemic blasts, as well as bone marrow-derived cells).

Experimental results show that the CAR design of the present invention provides CAR.CD19 T cells able to recognize and kill the potential generation of unwanted CAR+ leukemia cells.

In particular, in vitro studies showed that CD19+ leukemic cells transduced with the CAR.CD19 (iCas9CAR.CD19SL-LH) of the present invention, which is characterized by a short liker (SL) vector, besides having a long hinge (LH), show a reduced but not null expression of the target antigen CD19, allowing to be recognized by CAR.CD19 T cells in vitro and in vivo.

The experimental results show that CAR.CD19 of the present invention allows to control CAR+ leukemic cells in both in vitro and in vivo experiments, even when the production starts from biological material characterized by heavy contamination of leukemia blasts.

According to the present invention, the impact of high percentage of leukemia contamination in patient-derived starting material (SM) on CAR T cell drug product (DP) has been also evaluated. The results showed that although the presence of large number of CD19+ cells in SM did not affect transduction level of DPs, as well as the yield of final recover when SM had more than 45% of CD19+ B leukemic cell contamination. DPs were deeply characterized by cytofluorimetric-assay and molecular biology for Immunoglobulin (Ig)-rearrangements, showing that level of B cell contamination in DPs did not correlate with the percentage of CD19+ cells in SM.

Therefore, evidence is provided that the use of patient derived material highly enriched in B leukemic cells led to the generation of CAR T-cell products with a significant contamination of leukemic cells.

According to the present invention, peripheral blood mononuclear cells isolated from patients with a high percentage of circulating blasts (at either diagnosis or relapse to increase the likelihood of high level of B cell contamination in the starting materials) were genetically modified with a y-retroviral vector carrying a second-generation CAR.CD19.41bb molecule. By applying quantitative PCR for Ig rearrangements (molecular MRD), B-cell contamination in 50% of the CAR T-cell products has been observed, in the absence of any statistical correlation between MRD in the DPs and the percentage of CD19+ leukemic cells present in the starting material. In a CAR T-cell sample with a detection level of B cells below the sensitivity of the molecular assay, the EuroFlow flow-cytometry platform was indeed able to detect the presence of a significant percentage of contaminating B cells, that were also stained positive for the presence of CAR.CD19.

According to the present invention, as shown in the example, B leukemia cell lines genetically modified with CAR.CD19 vector with a short linker show a significant reduction in the CD19 MFI, with CD19 still detectable by cytofluorimetric assay, respect to what observed in B cell line expressing CAR.CD19 with the long liker, showing a complete negative binding of CD19 antibody.

Functional analysis confirmed these findings:

CAR.CD19 T cells of the invention were able to eliminate CAR+ leukemia cell lines in in vitro co-culture.

Finally, in vivo models corroborate the above-mentioned in vitro data.

CAR.CD19 according to the present invention can comprise also the suicide gene inducible caspase 9 (iC9) in the construct.

According to the present invention, a γ-retroviral vector coding for iC9.CAR.CD19 has been used, which is a bicistronic vector cloned in frame with the suicide gene iC9.

Therefore, CAR+ B cell leukemia could be in vivo controlled by either the systemic infusion of CAR.CD19 T cells or by the administration of AP1903 to activate the suicide gene inducible caspase 9 (iC9).

In fact, AP1903 (Rimiducid) is an inert small bio-molecule, which is able to activate the iC9-mediated caspase cascade by inducing dimerization of the FK-binding protein domain of the construct¹⁰. In a pivotal study conducted in patients given a T-cell depleted allograft followed by post-transplant infusion of donor T cells transduced with iC9, it was shown that AP1903 administration can trigger chemically-induced dimerization and eliminate genetically modified T cells from both peripheral blood and central nervous system (CNS), leading to rapid resolution of GVHD and CRS. Thus, iC9 activation by a single dose of AP1903 produce both rapid and long-term control of T cells carrying the suicide gene¹¹.

As described in the example below, CD19+ tumor cell lines were genetically modified with the bicistronic vector coding for iC9.CAR.CD19 to reproduce a CAR+ leukemic clonotype. In vitro experiments showed that the activation of iC9 by exposition to AP1903 was able to eliminate CAR+ leukemic cells. In particular, cytofluorimetric analysis of iC9.CAR+ leukemic cell lines treated with AP1903 shows that the remaining cells surviving in culture were characterized by a strongly reduced expression of the CAR respect to un-treated cells, but still with a detectable CAR MFI threshold. A more sensitive molecular analysis reveals that iC9 activation was associated with a significant reduction of transgene detection among the residual cells after the treatment, with leukemia B cells surviving after the AP1903 treatment showing a residual low VCN threshold.

These data confirmed several both in vitro^{14,15} and in vivo evidences¹⁶ showing that AP1903 treatment is highly efficient in eliminating the great majority of iC9+ cells, while sparing cells with a lower level of iC9 expression. Indeed, in the context of genetically modified T cells, patients who received a haploidentical iC9-T cells infusion after HSCT to treat GVHD, showed 1% of residual iC9-T cells after AP1903 administration. The residual iC9+ T cells were characterized by a remarkable dim expression of the transgene, possibly related to a low level of T cell activation. Indeed, iC9+ T cell elimination could be enhanced by T-cell activation during repeated AP1903 administrations¹⁶. By contrast, when iC9 is expressed in tumor cells, AP1903 is able to induce massive and rapid apoptosis, leading to a significant in vivo tumor control_{14,15}.

In the context of the present invention, in case of an unlikely event of genetic modification of leukemia cells by retroviral vector encoding a iC9.CAR.CD19 gene, the leukemia cells surviving to AP1903 exposure will lack high CAR expression on their surface, resulting in the possibility to target the CD19 antigen with high efficiency by both CAR.CD19 allogenic NK cells and CAR.CD19 T-cells, at the same extend of WT leukemia/lymphoma cells.

In addition, in the context of the present invention, CAR design is also a relevant factor regulating the ability of CAR.CD19 T-cells to recognize and kill CAR+ leukemia cells, without substantially modify the anti-leukemic activity of CAR.CD19 T cells. Indeed, CAR.CD19SL/LH or SL/SH T cells exert a significant anti-leukemia activity in both in vitro and in vivo models, at the same extent of the more conventional CAR.CD19LL/SH T cells. Nevertheless, when the CD19 and the short-linker CAR.CD19 are expressed in CIS on the same cellular membrane of leukemia B-cell lines, these latter showed significantly reduced CD19 MFI respect to wild-type B cells, with CD19 being still detectable by flow-cytometry, suggesting a non complete CIS masking of the antigen.

It is therefore specific object of the present invention a chimeric antigen receptor comprising or consisting of, from the N-terminus to the C-terminus:
a) a signal peptide,
b) a single chain antibody domain chosen from the group consisting of anti CD19 single chain antibody domain, anti CD20 single chain antibody domain or anti CD22 single chain antibody domain, said single chain antibody domain comprising or consisting of VL and VH sequences linked each other by a linker,
c) a hinge,
d) a trans membrane domain,
e) a co-stimulatory signaling domain, and
f) CD3Zeta chain sequence,
wherein said linker is a short flexible linker with a length from 7 to 14 (i.e., with a length of 7, 8, 9, 10, 11, 12, 13 or 14 amino acids), for example from 7 to 12 or from 7 to 10 or 8 amino acids.

Unless specified, as used herein, an scFv may have the VL and VH variable regions in either order, e.g. with respect to the N-terminal and C-terminal ends of the polypeptide, the scFv may comprise VL-linker-VH or may comprise VH-linker-VL.

According to the present invention, anti CD19 single chain antibody domain can comprise anti CD19 FMC63 hybridoma VL and VH sequences, in either order, wherein anti CD19 FMC63 hybridoma VL sequence comprises CDR1 sequence QDISKY (SEQ ID NO:1), CDR2 sequence HTS and CDR3 sequence GNTLP (SEQ ID NO:2), whereas anti CD19 FMC63 hybridoma VH sequence comprises CDR1 sequence GVSLPDYG (SEQ ID NO:3), CDR2 sequence IWGSETT (SEQ ID NO:4) and CDR3 sequence AKHYYYGGSYAMDY (SEQ ID NO:5);

anti CD20 single chain antibody domain can comprise anti CD20 VL and VH sequences, wherein anti CD20 VL sequence²² comprises CDR1 sequence SSVSY (SEQ ID NO:6), CDR2 sequence ATS and CDR3 sequence QQWTSNPPT (SEQ ID NO:7), whereas anti CD20 VH sequence comprises CDR1 sequence GYTFTSYN (SEQ ID NO:8), CDR2 sequence IYPGNGDT (SEQ ID NO:9) and CDR3 sequence ARSTYYGGDWYFNV (SEQ ID NO:10);

anti CD22 single chain antibody domain can comprise anti CD22 VL and VH sequences, wherein anti CD22 VL sequence comprises CDR1 sequence QSLANSYGNTF (SEQ ID NO:11), CDR2 sequence GIS and CDR3 sequence LQGTHQP (SEQ ID NO:12), whereas anti CD 22 VH sequence comprises CDR1 sequence GYRFTNYWIH (SEQ ID NO:13), CDR2 sequence INPGNNYA (SEQ ID NO:14) and CDR3 sequence TR.

According to the present invention, anti-CD19 FMC63 hybridoma VL sequence can comprise or consist of sequence

DIQMTQTTSSLSASLGDRVTISCRASQDISKYLNWYQQKPDGTVK LLIYHTSRLHSGVPSRFSGSGSGTDYSLTISNLEQEDIATYFCQQGNTLP YTFGGGTKLEIT (SEQ ID NO:15) and

anti-CD19 FMC63 hybridoma VH sequence can comprise or consist of sequence

EVKLQESGPGLVAPSQSLSVTCTVSGVSLPDYGVSWIRQPPRKG LEWLGVIWGSETTYYNSALKSRLTIIKDNSKSQVFLKMNSLQTDDTAIYYC AKHYYYGGSYAMDYWGQGTSVTVSS (SEQ ID NO:16);

anti-CD20 VL sequence can comprise or consist of sequence

QIVLSQSPAILSASPGEKVTMTCRASSSVSYIHWFQQKPGSSPKP WIYATSNLASGVPVRFSGSGSGTSYSLTISRVEAEDAATYYCQQWTSNP PTFGGGTKLEIK (SEQ ID NO:17) and

anti-CD20 VH sequence can comprise or consist of sequence

QVQLQQPGAELVKPGASVKMSCKASGYTFTSYNMHWVKQTPGR GLEWIGAIYPGNGDTSYNQKFKGKATLTADKSSSTAYMQLSSLTSEDSA VYYCARSTYYGGDWYFNVWGAGTTVTVSA (SEQ ID NO:18);

anti-CD22 VL sequence can comprise or consist of sequence

DVQVTQSPSSLSASVGDRVTITCRSSQSLANSYGNTFLSWYLHK PGKAPQLLIYGISNRFSGVPDRFSGSGSGTDFTLTISSLOPEDFATYYCL QGTHQPYTFGQGTKVEIK (SEQ ID NO:19) and anti-CD22 VH sequence can comprise or consist of sequence

EVQLVQSGAEVKKPGASVKVSCKASGYRFTNYWIHWVRQAPGQ GLEWIGGINPGNNYATYRRKFQGRVTMTADTSTSTVYMELSSLRSEDTA VYYCTREGYGNYGAWFAYWGQGTLVTVSS (SEQ ID NO:20).

The linker which links VL and VH sequences can be chosen from the group consisting of a short and flexible aminoacid glycines-rich sequence, such as (G4S)2 linker GGGGSGGGG (SEQ ID NO:35), G4SG2 linker GGGGSGG (SEQ ID NO:37) or G3SG4 linker GGGSGGGG (SEQ ID NO: 38), SG4SG3 linker SGGGGSGGG (SEQ ID NO:186), (SG4)2 S linker SGGGGSGGGGS (SEQ ID NO:187), (SG4)2 SG linker SGGGGSGGGGSG (SEQ ID NO:188), (SG4)2 SG3 linker SGGGGSGGGGSGGG linker (SEQ ID NO:189), (SG4)2 SGGGGSGGGG (SEQ ID NO:190), (SG4)2 SG2 SGGGGSGGGGSGG (SEQ ID NO:191), preferably G3SG4 linker.

According to specific embodiments, said hinge can comprise or consist of one or more of the following hinges:

CD8stalk

TTTPAPRPPTPAPTIASQPLSLRPEACRPAAGGAVHTRGLDFACD (SEQ ID NO:21) (nucleotide ID NO: M12828.1 and Protein ID NO:

AAB04637.1);

Hinge CD28 EVMYPPPYLDNEKSNGTIIHVKGKHLCPSPLFPGPSKP (SEQ ID NO:22) (nucleotide ID NO: AJ517504.1 and Protein ID NO: CAD57003.1);

hinge CH2-CH3 (UNIPROTKB: P01861)

ESKYGPPCPSCPAPEFLGGPSVFLFPPKPKDTLMISRTPEVTCVV VDVSQEDPEVQFNWYVDGVEVHNAKTKPREEQFNSTYRVVSVLTVLHQ DWLNGKEYKCKVSNKGLPSSIEKTISKAKGQPREPQVYTLPPSQEEMTK NQVSLTCLVKGFYPSDIAVEWESNGQPENNYKTTPPVLDSDGSFFLYSR LTVDKSRWQEGNVFSCSVMHEALHNHYTQKSLSLSLGK (SEQ ID NO: 23); or

hinge CH3 (UNIPROTKB: P01861):

ESKYGPPCPSCPGQPREPQVYTLPPSQEEMTKNQVSLTCLVKGF YPSDIAVEWESNGQPENNYKTTPPVLDSDGSFFLYSRLTVDKSRWQEG NVFSCSVMHEALHNHYTQKSLSLSLGK (SEQ ID NO:24), preferably CD8stalk.

According to the present invention, the hinge can be linked to the single chain antibody domain by a second linker (or adapter).

According to the present invention, said hinge can be linked, at the N terminus, to a trackable marker, said trackable marker being linked, optionally by a second linker (or adapter), to the single chain antibody domain, i.e. the second linker links the single chain antibody domain and the trackable marker. For example, the second linker (or adapter) can be a dipeptide, such as for example GS.

According to the present invention the trackable marker can be chosen from the group consisting of:

ΔCD34 ELPTQGTFSNVSTNVS (SEQ ID NO:25) (nucleotide ID NO AB238231.1 and Protein ID NO: BAE46748.1); or

NGFR

KEACPTGLYTHSGECCKACNLGEGVAQPCGANQTVCEPCLDSV TFSDVVSATEPCKPCTECVGLQSMSAPCVEADDAVCRCAYGYYQDETT GRCEACRVCEAGSGLVFSCQDKQNTVCEECPDGTYSDEANHVDPCLP CTVCEDTERQLRECTRWADAECEEIPGRWITRSTPPEGSDSTAPSTQEP EAPPEQDLIASTVAGVVTTVMGSSQPVVTRGTTDN (SEQ ID NO:26) (nucleotide ID NO:AK313654.1 and Protein ID NO:BAG36408.1), preferably ΔCD34.

According to the present invention, the hinge CD8stalk can be linked to the trackable marker ΔCD34.

The trans membrane domain can be chosen from the group consisting of CD28TM FWVLVVVGGVLACYSLLVTVAFIIFWV (SEQ ID NO: 27) (nucleotide ID NO: BC112085.1 and Protein ID NO: AAl12086.1) or CD8aTM IYIWAPLAGTCGVLLLSLVIT (SEQ ID NO:28) (nucleotide ID NO NM_001768.6 and Protein ID NO: NP_001759.3), preferably CD8aTM.

According to the present invention the co-stimulatory signaling domain can be chosen from the group consisting of

CD28 cytoplasmic sequence RSKRSRLLHSDYMNMTPRRPGPTRKHYQPYAPPRDFAAYRS (SEQ ID NO: 29) (nucleotide ID NO: AF222341.1 and Protein ID NO: AAF33792.1),

CD137 (4-1BB) sequence KRGRKKLLYIFKQPFMRPVQTTQEEDGCSCRFPEEEEGGCEL (SEQ ID NO: 30) (nucleotide ID NO: U03397.1 and Protein NO: AAA53133.1),

OX40 sequence RDQRLPPDAHKPPGGGSFRTPIQEEQADAHSTLAKI (SEQ ID NO:31) (nucleotide ID NO: NM_003327.3 and Protein NO: NP_003318.1), or

a sequence obtained by linking:

CD28 cytoplasmic sequence (SEQ ID NO:29) with CD137 (4-1BB) sequence (SEQ ID NO:30),

CD137 (4-1BB) sequence (SEQ ID NO:30) with CD28 cytoplasmic sequence (SEQ ID NO:29),

CD28 cytoplasmic sequence (SEQ ID NO:29) with OX40 sequence (SEQ ID NO:31),

OX40 sequence (SEQ ID NO:31) with CD28 cytoplasmic sequence (SEQ ID NO:29),

OX40 sequence (SEQ ID NO:31) with CD137 (4-1BB) sequence (SEQ ID NO:30),

CD137 (4-1BB) sequence (SEQ ID NO:30) with OX40 sequence (SEQ ID NO:31)

According to the present invention, CD3Zeta chain sequence is RVKFSRSADAPAYQQGQNQLYNELNLGRREEYDVLDKRRGRDPEMGG KPRRKNPQEGLYNELQKDKMAEAYSEIGMKGERRRGKGHDGLYQGLS TATKDTYDALHMQALPPR* (SEQ ID NO:32) (nucleotide ID NO: J04132.1 and Protein ID NO: AAA60394.1).

According to the present invention, the chimeric antigen receptor can further comprise cytoplasmic moiety of CD8cyt: LYCNHRNRRRVCKCPR (SEQ ID NO:40) (nucleotide ID NO NM_001768.6 and Protein ID NO: NP_001759.3) between the transmembrane domain and the co-stimulatory signaling domain.

The cytoplasmic moiety of CD8cyt can be linked to the co-stimulatory signaling domain by a linker, such as a dipeptide, for example VD.

According to the present invention, the signal peptide can comprise or consist of MEFGLSWLFLVAILKGVQC (SEQ ID NO:41) (nucleotide ID NO: AB776838.1 and Protein ID NO: BAN63131.1).

According to the present invention, the chimeric antigen receptor comprises or consists of

(SEQ ID NO: 72)
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNL
EQEDIATYFCQQGNTLPYTFGGGTKLEITGGGSGGGGEVKLQESGPGL
VAPSQSLSVTCTVSGVSLPDYGVSWIRQPPRKGLEWLGVIWGSETTYY
NSALKSRLTIIKDNSKSQVFLKMNSLQTDDTAIYYCAKHYYYGGSYAMDY
WGQGTSVTVSSGSELPTQGTFSNVSTNVSPAPRPPTPAPTIASQPLSLR
PEACRPAAGGAVHTRGLDFACDIYIWAPLAGTCGVLLLSLVITLYCNHRN
RRRVCKCPRVDKRGRKKLLYIFKQPFMRPVQTTQEEDGCSCRFPEEEE
GGCELRVKFSRSADAPAYQQGQNQLYNELNLGRREEYDVLDKRRGRD
PEMGGKPRRKNPQEGLYNELQKDKMAEAYSEIGMKGERRRGKGHDGL
YQGLSTATKDTYDALHMQALPPR
or
(SEQ ID NO: 33)
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNL
EQEDIATYFCQQGNTLPYTFGGGTKLEITGGGSGGGGEVKLQESGPGL
VAPSQSLSVTCTVSGVSLPDYGVSWIRQPPRKGLEWLGVIWGSETTYY
NSALKSRLTIIKDNSKSQVFLKMNSLQTDDTAIYYCAKHYYYGGSYAMDY
WGQGTSVTVSSGSPAPRPPTPAPTIASQPLSLRPEACRPAAGGAVHTR
GLDFACDIYIWAPLAGTCGVLLLSLVITLYCNHRNRRRVCKCPRVDKRGR
KKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGL
YNELQKDKMAEAYSEIGMKGERRRGKGHDGLYQGLSTATKDTYDALHM
QALPPR.

Namely, anti-CD19 chimeric antigen receptor according to the present invention can consist of:

signal peptide MEFGLSWLFLVAILKGVQC (SEQ ID NO:41), which is linked by a SR linker to

anti CD19 single chain antibody domain from FMC63 hybridoma consisting of FMC63 VL sequence DIQMTQTTSSLSASLGDRVTISCRASQDISKYLNWYQQKPDGTVKLLIYH TSRLHSGVPSRFSGSGSGTDYSLTISNLEQEDIATYFCQQGNTLPYTFG GGTKLEIT (SEQ ID NO:15) linked by Flex Linker (short linker-SL), preferably G3SG4 linker GGGSGGGG (SEQ ID NO:38), to FMC63 VH sequence EVKLQESGPGLVAPSQSLSVTCTVSGVSLPDYGVSWIRQPPRKGLEWL GVIWGSETTYYNSALKSRLTIIKDNSKSQVFLKMNSLQTDDTAIYYCAKHY YYGGSYAMDYWGQGTSVTVSS (SEQ ID NO:16), FMC63 VH sequence being linked by the GS linker (i.e. the second linker or adapter) to a:

Trackable marker ACD34 ELPTQGTFSNVSTNVS (SEQ ID NO:25) and CD8stalk PAPRPPTPAPTIASQPLSLRPEACRPAAGGAVHTRGLDFACD (SEQ ID NO: 21) (Long hinge-LH: ΔCD34+ CD8stalk) or CD8stalk PAPRPPTPAPTIASQPLSLRPEACRPAAGGAVHTRGLDFACD (SEQ ID NO: 21) (Short Hinge-SH: CD8stalk), which is linked to

CD8aTM IYIWAPLAGTCGVLLLSLVIT (SEQ ID NO:28), which is linked to

cytoplasmic moiety of CD8cyt LYCNHRNRRRVCKCPR (SEQ ID NO: 40), which is linked by linker VD to

co-stimulatory signaling domain CD137 (4-1BB) sequence: KRGRKKLLYIFKQPFMRPVQTTQEEDGCSCRFPEEEEGGCEL (SEQ ID NO: 30), which is linked to CD3Zeta chain RVKFSRSADAPAYQQGQNQLYNELNLGRREEYDVLDKRRGRDPEMGG KPRRKNPQEGLYNELQKDKMAEAYSEIGMKGERRRGKGHDGLYQGLS TATKDTYDALHMQALPPR* (SEQ ID NO:32).

The present invention concerns also a nucleotide sequence comprising or consisting of a nucleotide sequence which encodes a chimeric antigen receptor as defined above.

In particular, anti CD19 FMC63 hybridoma VL sequence can be encoded by the nucleotide sequence

(SEQ ID NO: 52)
GACATCCAGATGACACAGACTACATCCTCCCTGTCTGCCTCTC
TGGGAGACAGAGTCACCATCAGTTGCAGGGCAAGTCAGGACATTAGT
AAATATTTAAATTGGTATCAGCAGAAACCAGATGGAACTGTTAAACTC
CTGATCTACCATACATCAAGATTACACTCAGGAGTCCCATCAAGGTTC
AGTGGCAGTGGGTCTGGAACAGATTATTCTCTCACCATTAGCAACCT
GGAGCAAGAAGATATTGCCACTTACTTTTGCCAACAGGGTAATACGC
TTCCGTACACGTTCGGAGGGGGGACTAAGTTGGAAATAACA

and

anti CD19 FMC63 hybridoma VH sequence can be encoded by the nucleotide sequence

(SEQ ID NO: 53)
GAGGTGAAACTGCAGGAGTCAGGACCTGGCCTGGTGGCGCC
CTCACAGAGCCTGTCCGTCACATGCACTGTCTCAGGGGTCTCATTAC
CCGACTATGGTGTAAGCTGGATTCGCCAGCCTCCACGAAAGGGTCTG
GAGTGGCTGGGAGTAATATGGGGTAGTGAAACCACATACTATAATTC
AGCTCTCAAATCCAGACTGACCATCATCAAGGACAACTCCAAGAGCC
AAGTTTTCTTAAAAATGAACAGTCTGCAAACTGATGACACAGCCATTT
ACTACTGTGCCAAACATTATTACTACGGTGGTAGCTATGCTATGGACT
ACTGGGGTCAAGGAACCTCAGTCACCGTCTCCTCA;

anti CD20 VL sequence can be encoded by the nucleotide sequence

(SEQ ID NO: 54)
CAGATCGTGCTGAGCCAGAGCCCCGCCATCCTGAGCGCCAGC
CCCGGCGAGAAGGTGACCATGACCTGCAGGGCCAGCAGCAGCGTG
AGCTACATCCACTGGTTCCAGCAGAAGCCCGGCAGCAGCCCCAAGC
CCTGGATCTACGCCACCAGCAACCTGGCCAGCGGCGTGCCCGTGAG
GTTCAGCGGCAGCGGCAGCGGCACCAGCTACAGCCTGACCATCAGC
AGGGTGGAGGCCGAGGACGCCGCCACCTACTACTGCCAGCAGTGGA
CCAGCAACCCCCCCACCTTCGGCGGCGGCACCAAGCTGGAGATCAA
G

and

anti CD20 VH sequence can be encoded by the nucleotide sequence

(SEQ ID NO: 55)
CAGGTGCAGCTGCAGCAGCCCGGCGCCGAGCTGGTGAAGCC
CGGCGCCAGCGTGAAGATGAGCTGCAAGGCCAGCGGCTACACCTTC
ACCAGCTACAACATGCACTGGGTGAAGCAGACCCCCGGCAGGGGCC
TGGAGTGGATCGGCGCCATCTACCCCGGCAACGGCGACACCAGCTA
CAACCAGAAGTTCAAGGGCAAGGCCACCCTGACCGCCGACAAGAGC
AGCAGCACCGCCTACATGCAGCTGAGCAGCCTGACCAGCGAGGACA
GCGCCGTGTACTACTGCGCCAGGAGCACCTACTACGGCGGCGACTG
GTACTTCAACGTGTGGGGCGCCGGCACCACCGTGACCGTGAGC;

anti CD22 VL sequence can be encoded by the nucleotide sequence

(SEQ ID NO: 56)
GACGTGCAGGTGACCCAGAGCCCCAGCAGCCTGAGCGCCAGCGTG
GGCGACAGGGTGACCATCACCTGCAGGAGCAGCCAGAGCCTGGCCA
ACAGCTACGGCAACACCTTCCTGAGCTGGTACCTGCACAAGCCCGG
CAAGGCCCCCCAGCTGCTGATCTACGGCATCAGCAACAGGTTCAGC
GGCGTGCCCGACAGGTTCAGCGGCAGCGGCAGCGGCACCGACTTC
ACCCTGACCATCAGCAGCCTGCAGCCCGAGGACTTCGCCACCTACTA
CTGCCTGCAGGGCACCCACCAGCCCTACACCTTCGGCCAGGGCACC
AAGGTGGAGATCAAG

and

anti CD22 VH sequence can be encoded by the nucleotide sequence

(SEQ ID NO: 57)
GAGGTGCAGCTGGTGCAGAGCGGCGCCGAGGTGAAGAAGCCCGGC
GCCAGCGTGAAGGTGAGCTGCAAGGCCAGCGGCTACAGGTTCACCA
ACTACTGGATCCACTGGGTGAGGCAGGCCCCCGGCCAGGGCCTGGA
GTGGATCGGCGGCATCAACCCCGGCAACAACTACGCCACCTACAGG
AGGAAGTTCCAGGGCAGGGTGACCATGACCGCCGACACCAGCACCA
GCACCGTGTACATGGAGCTGAGCAGCCTGAGGAGCGAGGACACCGC
CGTGTACTACTGCACCAGGGAGGGCTACGGCAACTACGGCGCCTGG
TTCGCCTACTGGGGCCAGGGCACCCTGGTGACCGTGAGCAGC.

According to the present invention, the nucleotide sequence encoding anti-CD19 chimeric antigen receptor is:

(SEQ ID NO: 58)
ATGGAGTTTGGACTTTCTTGGTTGTTTTTGGTGGCAATTCTGAAGGGT
GTCCAGTGTAGCAGGGACATCCAGATGACACAGACTACATCCTCCCT
GTCTGCCTCTCTGGGAGACAGAGTCACCATCAGTTGCAGGGCAAGTC
AGGACATTAGTAAATATTTAAATTGGTATCAGCAGAAACCAGATGGAA
CTGTTAAACTCCTGATCTACCATACATCAAGATTACACTCAGGAGTCC
CATCAAGGTTCAGTGGCAGTGGGTCTGGAACAGATTATTCTCTCACC
ATTAGCAACCTGGAGCAAGAAGATATTGCCACTTACTTTTGCCAACAG
GGTAATACGCTTCCGTACACGTTCGGAGGGGGGACTAAGTTGGAAAT
AACAGGCGGAGGAAGCGGAGGTGGGGGCGAGGTGAAACTGCAGGA
GTCAGGACCTGGCCTGGTGGCGCCCTCACAGAGCCTGTCCGTCACA
TGCACTGTCTCAGGGGTCTCATTACCCGACTATGGTGTAAGCTGGAT
TCGCCAGCCTCCACGAAAGGGTCTGGAGTGGCTGGGAGTAATATGG
GGTAGTGAAACCACATACTATAATTCAGCTCTCAAATCCAGACTGACC
ATCATCAAGGACAACTCCAAGAGCCAAGTTTTCTTAAAAATGAACAGT
CTGCAAACTGATGACACAGCCATTTACTACTGTGCCAAACATTATTAC
TACGGTGGTAGCTATGCTATGGACTACTGGGGTCAAGGAACCTCAGT
CACCGTCTCCTCAGGATCCGAACTTCCTACTCAGGGGACTTTCTCAA
ACGTTAGCACAAACGTAAGTCCCGCCCCAAGACCCCCCACACCTGC
GCCGACCATTGCTTCTCAACCCCTGAGTTTGAGACCCGAGGCCTGCC
GGCCAGCTGCCGGCGGGGCCGTGCATACAAGAGGACTCGATTTCGC
TTGCGACATCTATATCTGGGCACCTCTCGCTGGCACCTGTGGAGTCC
TTCTGCTCAGCCTGGTTATTACTCTGTACTGTAATCACCGGAATCGCC
GCCGCGTTTGTAAGTGTCCCAGGGTCGACAAACGGGGCAGAAAGAA
ACTCCTGTATATATTCAAACAACCATTTATGAGACCAGTACAAACTACT
CAAGAGGAAGATGGCTGTAGCTGCCGATTTCCAGAAGAAGAAGAAG
GAGGATGTGAACTGAGAGTGAAGTTCAGCAGGAGCGCAGACGCCCC
CGCGTACCAGCAGGGCCAGAACCAGCTCTATAACGAGCTCAATCTAG
GACGAAGAGAGGAGTACGATGTTTTGGACAAGAGACGTGGCCGGGA
CCCTGAGATGGGGGGAAAGCCGAGAAGGAAGAACCCTCAGGAAGGC
CTGTACAATGAACTGCAGAAAGATAAGATGGCGGAGGCCTACAGTGA
GATTGGGATGAAAGGCGAGCGCCGGAGGGGCAAGGGGCACGATGG
CCTTTACCAGGGTCTCAGTACAGCCACCAAGGACACCTACGACGCCC
TTCACATGCAGGCCCTGCCCCCTCGCTAA
or
(SEQ ID NO: 34)
ATGGAGTTTGGACTTTCTTGGTTGTTTTTGGTGGCAATTCTGAAGGGT
GTCCAGTGTAGCAGGGACATCCAGATGACACAGACTACATCCTCCCT
GTCTGCCTCTCTGGGAGACAGAGTCACCATCAGTTGCAGGGCAAGTC
AGGACATTAGTAAATATTTAAATTGGTATCAGCAGAAACCAGATGGAA
CTGTTAAACTCCTGATCTACCATACATCAAGATTACACTCAGGAGTCC
CATCAAGGTTCAGTGGCAGTGGGTCTGGAACAGATTATTCTCTCACC
ATTAGCAACCTGGAGCAAGAAGATATTGCCACTTACTTTTGCCAACAG
GGTAATACGCTTCCGTACACGTTCGGAGGGGGGACTAAGTTGGAAAT
AACAGGCGGAGGAAGCGGAGGTGGGGGCGAGGTGAAACTGCAGGA
GTCAGGACCTGGCCTGGTGGCGCCCTCACAGAGCCTGTCCGTCACA
TGCACTGTCTCAGGGGTCTCATTACCCGACTATGGTGTAAGCTGGAT
TCGCCAGCCTCCACGAAAGGGTCTGGAGTGGCTGGGAGTAATATGG
GGTAGTGAAACCACATACTATAATTCAGCTCTCAAATCCAGACTGACC
ATCATCAAGGACAACTCCAAGAGCCAAGTTTTCTTAAAAATGAACAGT
CTGCAAACTGATGACACAGCCATTTACTACTGTGCCAAACATTATTAC
TACGGTGGTAGCTATGCTATGGACTACTGGGGTCAAGGAACCTCAGT
CACCGTCTCCTCAGGATCCCCCGCCCCAAGACCCCCCACACCTGCG
CCGACCATTGCTTCTCAACCCCTGAGTTTGAGACCCGAGGCCTGCCG
GCCAGCTGCCGGCGGGGCCGTGCATACAAGAGGACTCGATTTCGCT
TGCGACATCTATATCTGGGCACCTCTCGCTGGCACCTGTGGAGTCCT
TCTGCTCAGCCTGGTTATTACTCTGTACTGTAATCACCGGAATCGCCG
CCGCGTTTGTAAGTGTCCCAGGGTCGACAAACGGGGCAGAAAGAAA
CTCCTGTATATATTCAAACAACCATTTATGAGACCAGTACAAACTACTC
AAGAGGAAGATGGCTGTAGCTGCCGATTTCCAGAAGAAGAAGAAGGA
GGATGTGAACTGAGAGTGAAGTTCAGCAGGAGCGCAGACGCCCCCG
CGTACCAGCAGGGCCAGAACCAGCTCTATAACGAGCTCAATCTAGGA
CGAAGAGAGGAGTACGATGTTTTGGACAAGAGACGTGGCCGGGACC
CTGAGATGGGGGGAAAGCCGAGAAGGAAGAACCCTCAGGAAGGCCT
GTACAATGAACTGCAGAAAGATAAGATGGCGGAGGCCTACAGTGAGA
TTGGGATGAAAGGCGAGCGCCGGAGGGGCAAGGGGCACGATGGCC
TTTACCAGGGTCTCAGTACAGCCACCAAGGACACCTACGACGCCCTT
CACATGCAGGCCCTGCCCCCTCGCTAAA.

According to the present invention, the nucleotide sequence can further comprise a nucleotide sequence encoding a suicide gene inducible amino acid sequence linked to the nucleotide sequence encoding said chimeric antigen receptor by a nucleotide sequence encoding a 2A self-cleaving peptide. The suicide gene inducible amino acid sequence can be a chimeric Caspase-9 polypeptide or comprises a herpes simplex virus thymidine kinase.

Therefore, in the cell, the polynucleotide 2A self-cleaving peptide cut the peptide comprising the suicide gene inducible amino acid sequence and the chimeric antigen receptor and in two separate peptides, i.e. the suicide gene inducible and the chimeric antigen receptor amino acid sequences.

According to an embodiment, the nucleotide sequence can be:

(SEQ ID NO: 180)
ATGCTCGAGATGCTGGAGGGAGTGCAGGTGGAGACTATTAGC
CCCGGAGATGGCAGAACATTCCCCAAAAGAGGACAGACTTGCGTCG
TGCATTATACTGGAATGCTGGAAGACGGCAAGAAGGTGGACAGCAG
CCGGGACCGAAACAAGCCCTTCAAGTTCATGCTGGGGAAGCAGGAA
GTGATCCGGGGCTGGGAGGAAGGAGTCGCACAGATGTCAGTGGGAC
AGAGGGCCAAACTGACTATTAGCCCAGACTACGCTTATGGAGCAACC
GGCCACCCCGGGATCATTCCCCCTCATGCTACACTGGTCTTCGATGT
GGAGCTGCTGAAGCTGGAAAGCGGAGGAGGATCCGGAGTGGACGG
GTTTGGAGATGTGGGAGCCCTGGAATCCCTGCGGGGCAATGCCGAT
CTGGCTTACATCCTGTCTATGGAGCCTTGCGGCCACTGTCTGATCAT
TAACAATGTGAACTTCTGCAGAGAGAGCGGGCTGCGGACCAGAACA
GGATCCAATATTGACTGTGAAAAGCTGCGGAGAAGGTTCTCTAGTCT
GCACTTTATGGTCGAGGTGAAAGGCGATCTGACCGCTAAGAAAATGG
TGCTGGCCCTGCTGGAACTGGCTCGGCAGGACCATGGGGCACTGGA
TTGCTGCGTGGTCGTGATCCTGAGTCACGGCTGCCAGGCTTCACATC
TGCAGTTCCCTGGGGCAGTCTATGGAACTGACGGCTGTCCAGTCAG
CGTGGAGAAGATCGTGAACATCTTCAACGGCACCTCTTGCCCAAGTC
TGGGCGGGAAGCCCAAACTGTTCTTTATTCAGGCCTGTGGAGGCGA
GCAGAAAGATCACGGCTTCGAAGTGGCTAGCACCTCCCCCGAGGAC
GAATCACCTGGAAGCAACCCTGAGCCAGATGCAACCCCCTTCCAGGA
AGGCCTGAGGACATTTGACCAGCTGGATGCCATCTCAAGCCTGCCCA
CACCTTCTGACATTTTCGTCTCTTACAGTACTTTCCCTGGATTTGTGA
GCTGGCGCGATCCAAAGTCAGGCAGCTGGTACGTGGAGACACTGGA
CGATATCTTTGAGCAGTGGGCCCATTCTGAAGACCTGCAGAGTCTGC
TGCTGCGAGTGGCCAATGCTGTCTCTGTGAAGGGGATCTACAAACAG
ATGCCAGGATGCTTCAACTTTCTGAGAAAGAAACTGTTCTTTAAGACC
TCCGCATCTAGGGCCCCGCGGGAAGGCCGAGGGAGCCTGCTGACAT
GTGGCGATGTGGAGGAAAACCCAGGACCACCATGGATGGAGTTTGG
ACTTTCTTGGTTGTTTTTGGTGGCAATTCTGAAGGGTGTCCAGTGTAG
CAGGGACATCCAGATGACACAGACTACATCCTCCCTGTCTGCCTCTC
TGGGAGACAGAGTCACCATCAGTTGCAGGGCAAGTCAGGACATTAGT
AAATATTTAAATTGGTATCAGCAGAAACCAGATGGAACTGTTAAACTC
CTGATCTACCATACATCAAGATTACACTCAGGAGTCCCATCAAGGTTC
AGTGGCAGTGGGTCTGGAACAGATTATTCTCTCACCATTAGCAACCT
GGAGCAAGAAGATATTGCCACTTACTTTTGCCAACAGGGTAATACGC
TTCCGTACACGTTCGGAGGGGGGACTAAGTTGGAAATAACAGGCGG
AGGAAGCGGAGGTGGGGGCGAGGTGAAACTGCAGGAGTCAGGACC
TGGCCTGGTGGCGCCCTCACAGAGCCTGTCCGTCACATGCACTGTC
TCAGGGGTCTCATTACCCGACTATGGTGTAAGCTGGATTCGCCAGCC
TCCACGAAAGGGTCTGGAGTGGCTGGGAGTAATATGGGGTAGTGAA
ACCACATACTATAATTCAGCTCTCAAATCCAGACTGACCATCATCAAG
GACAACTCCAAGAGCCAAGTTTTCTTAAAAATGAACAGTCTGCAAACT
GATGACACAGCCATTTACTACTGTGCCAAACATTATTACTACGGTGGT
AGCTATGCTATGGACTACTGGGGTCAAGGAACCTCAGTCACCGTCTC
CTCAGGATCCGAACTTCCTACTCAGGGGACTTTCTCAAACGTTAGCA
CAAACGTAAGTCCCGCCCCAAGACCCCCCACACCTGCGCCGACCAT
TGCTTCTCAACCCCTGAGTTTGAGACCCGAGGCCTGCCGGCCAGCT
GCCGGCGGGGCCGTGCATACAAGAGGACTCGATTTCGCTTGCGACA
TCTATATCTGGGCACCTCTCGCTGGCACCTGTGGAGTCCTTCTGCTC
AGCCTGGTTATTACTCTGTACTGTAATCACCGGAATCGCCGCCGCGT
TTGTAAGTGTCCCAGGGTCGACAAACGGGGCAGAAAGAAACTCCTGT
ATATATTCAAACAACCATTTATGAGACCAGTACAAACTACTCAAGAGG
AAGATGGCTGTAGCTGCCGATTTCCAGAAGAAGAAGAAGGAGGATGT
GAACTGAGAGTGAAGTTCAGCAGGAGCGCAGACGCCCCCGCGTACC
AGCAGGGCCAGAACCAGCTCTATAACGAGCTCAATCTAGGACGAAGA
GAGGAGTACGATGTTTTGGACAAGAGACGTGGCCGGGACCCTGAGA
TGGGGGGAAAGCCGAGAAGGAAGAACCCTCAGGAAGGCCTGTACAA
TGAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCACAT
GCAGGCCCTGCCCCCTCGCTAA.

Namely, this nucleotide sequence, which encodes the sequence named also as iCas9CAR.CD19SL-LH, comprises the following sequences:

iCas9 chimeric protein (FKBP12wt binding region-linker-Caspase-9 polypeptide):
FKBP12wt binding region:
(SEQ ID NO: 59)
ATGCTCGAGATGCTGGAGGGAGTGCAGGTGGAGACTATTAGC
CCCGGAGATGGCAGAACATTCCCCAAAAGAGGACAGACTTGCGTCG
TGCATTATACTGGAATGCTGGAAGACGGCAAGAAGGTGGACAGCAG
CCGGGACCGAAACAAGCCCTTCAAGTTCATGCTGGGGAAGCAGGAA
GTGATCCGGGGCTGGGAGGAAGGAGTCGCACAGATGTCAGTGGGAC
AGAGGGCCAAACTGACTATTAGCCCAGACTACGCTTATGGAGCAACC
GGCCACCCCGGGATCATTCCCCCTCATGCTACACTGGTCTTCGATGT
GGAGCTGCTGAAGCTGGAA (nucleotide ID NO: BT007066.1)
Link of connection
(SEQ ID NO: 60)
AGCGGAGGAGGATCCGGA
Caspase-9 polypeptide
(SEQ ID NO: 61)
GTGGACGGGTTTGGAGATGTGGGAGCCCTGGAATCCCTGCGG
GGCAATGCCGATCTGGCTTACATCCTGTCTATGGAGCCTTGCGGCCA
CTGTCTGATCATTAACAATGTGAACTTCTGCAGAGAGAGCGGGCTGC
GGACCAGAACAGGATCCAATATTGACTGTGAAAAGCTGCGGAGAAGG
TTCTCTAGTCTGCACTTTATGGTCGAGGTGAAAGGCGATCTGACCGC
TAAGAAAATGGTGCTGGCCCTGCTGGAACTGGCTCGGCAGGACCAT
GGGGCACTGGATTGCTGCGTGGTCGTGATCCTGAGTCACGGCTGCC
AGGCTTCACATCTGCAGTTCCCTGGGGCAGTCTATGGAACTGACGGC
TGTCCAGTCAGCGTGGAGAAGATCGTGAACATCTTCAACGGCACCTC
TTGCCCAAGTCTGGGCGGGAAGCCCAAACTGTTCTTTATTCAGGCCT
GTGGAGGCGAGCAGAAAGATCACGGCTTCGAAGTGGCTAGCACCTC
CCCCGAGGACGAATCACCTGGAAGCAACCCTGAGCCAGATGCAACC
CCCTTCCAGGAAGGCCTGAGGACATTTGACCAGCTGGATGCCATCTC
AAGCCTGCCCACACCTTCTGACATTTTCGTCTCTTACAGTACTTTCCC
TGGATTTGTGAGCTGGCGCGATCCAAAGTCAGGCAGCTGGTACGTG
GAGACACTGGACGATATCTTTGAGCAGTGGGCCCATTCTGAAGACCT
GCAGAGTCTGCTGCTGCGAGTGGCCAATGCTGTCTCTGTGAAGGGG
ATCTACAAACAGATGCCAGGATGCTTCAACTTTCTGAGAAAGAAACTG
TTCTTTAAGACCTCC (nucleotide ID NO: AK292111.1)
Link of connection
(SEQ ID NO: 62)
GCATCTAGGGCCCCGCGG
T2A self-cleaving peptides
(SEQ ID NO: 63)
GAAGGCCGAGGGAGCCTGCTGACATGTGGCGATGTGGAGGA
AAACCCAGGACCA (nucleotide ID NO: AF062037.1)
Short linker of connection
CCATGG
Signal peptide
(SEQ ID NO: 64)
ATGGAGTTTGGACTTTCTTGGTTGTTTTTGGTGGCAATTCTGAA
GGGTGTCCAGTGTAGCAGG (nucleotide ID NO: AB776838.1)
VL sequence (FMC63)
(SEQ ID NO: 52)
GACATCCAGATGACACAGACTACATCCTCCCTGTCTGCCTCTC
TGGGAGACAGAGTCACCATCAGTTGCAGGGCAAGTCAGGACATTAGT
AAATATTTAAATTGGTATCAGCAGAAACCAGATGGAACTGTTAAACTC
CTGATCTACCATACATCAAGATTACACTCAGGAGTCCCATCAAGGTTC
AGTGGCAGTGGGTCTGGAACAGATTATTCTCTCACCATTAGCAACCT
GGAGCAAGAAGATATTGCCACTTACTTTTGCCAACAGGGTAATACGC
TTCCGTACACGTTCGGAGGGGGGACTAAGTTGGAAATAACA
Flex short Linker
(SEQ ID NO: 65)
GGCGGAGGAAGCGGAGGTGGGGGC
VH sequence (FMC63)
(SEQ ID NO: 53)
GAGGTGAAACTGCAGGAGTCAGGACCTGGCCTGGTGGCGCC
CTCACAGAGCCTGTCCGTCACATGCACTGTCTCAGGGGTCTCATTAC
CCGACTATGGTGTAAGCTGGATTCGCCAGCCTCCACGAAAGGGTCTG
GAGTGGCTGGGAGTAATATGGGGTAGTGAAACCACATACTATAATTC
AGCTCTCAAATCCAGACTGACCATCATCAAGGACAACTCCAAGAGCC
AAGTTTTCTTAAAAATGAACAGTCTGCAAACTGATGACACAGCCATTT
ACTACTGTGCCAAACATTATTACTACGGTGGTAGCTATGCTATGGACT
ACTGGGGTCAAGGAACCTCAGTCACCGTCTCCTCA
LH (Long Hinge): (short linker, i.e. the second linker or adapter) +
(trackable marker: ΔCD34 extracellular + hinge: CD8stalk extracellular):
Short adapter
GGATCC
ΔCD34 extracellular
(SEQ ID NO: 66)
GAACTTCCTACTCAGGGGACTTTCTCAAACGTTAGCACAAACG
TAAGT (nucleotide ID NO AB238231.1)
CD8stalk extracellular
(SEQ ID NO: 67)
CCCGCCCCAAGACCCCCCACACCTGCGCCGACCATTGCTTCT
CAACCCCTGAGTTTGAGACCCGAGGCCTGCCGGCCAGCTGCCGGCG
GGGCCGTGCATACAAGAGGACTCGATTTCGCTTGCGAC
(nucleotide ID NO: M12828.1);
CD8aTM (transmembrane)
(SEQ ID NO: 68)
ATCTATATCTGGGCACCTCTCGCTGGCACCTGTGGAGTCCTTC
TGCTCAGCCTGGTTATTACT (nucleotide ID NO NM_001768.6)
CD8cyt (Cytoplasmic)
(SEQ ID NO: 69)
CTGTACTGTAATCACCGGAATCGCCGCCGCGTTTGTAAGTGTC
CCAGG (nucleotide ID NO NM_001768)
Short linker (containing Sal I site)
GTCGAC
4-1BB
(SEQ ID NO: 70)
AAACGGGGCAGAAAGAAACTCCTGTATATATTCAAACAACCATT
TATGAGACCAGTACAAACTACTCAAGAGGAAGATGGCTGTAGCTGCC
GATTTCCAGAAGAAGAAGAAGGAGGATGTGAACTG
(nucleotide ID NO: U03397.1)
CD3Zeta chain
(SEQ ID NO: 71)
AGAGTGAAGTTCAGCAGGAGCGCAGACGCCCCCGCGTACCAG
CAGGGCCAGAACCAGCTCTATAACGAGCTCAATCTAGGACGAAGAGA
GGAGTACGATGTTTTGGACAAGAGACGTGGCCGGGACCCTGAGATG
GGGGGAAAGCCGAGAAGGAAGAACCCTCAGGAAGGCCTGTACAATG
AACTGCAGAAAGATAAGATGGCGGAGGCCTACAGTGAGATTGGGAT
GAAAGGCGAGCGCCGGAGGGGCAAGGGGCACGATGGCCTTTACCA
GGGTCTCAGTACAGCCACCAAGGACACCTACGACGCCCTTCACATGC
AGGCCCTGCCCCCTCGCTAA (nucleotide ID NO: J04132.1).

According to the present invention iCas9CAR.CD19SL-SH consists of the sequence of iCas9CAR.CD19SL-LH with the exception of the fact that it comprises a short hinge (SH): (short adapter)+(Hinge: CD8stalk extracellular).

The present invention concerns also a vector comprising the nucleotide sequence as defined above, wherein said vector is a DNA vector, a RNA vector, a plasmid, a lentivirus vector, adenoviral vector, retrovirus vector, such as y-retroviral vector, or non viral vector.

In addition, the present invention concerns a cell, such as T cell, such as alfa/beta and gamma/delta T cell, NK cells, NK-T cells, comprising the chimeric antigen receptor as defined above and/or the vector or plasmid as defined above.

The cell according to the present invention can further comprise a suicide gene inducible amino acid sequence such as a chimeric Caspase-9 polypeptide or comprises a herpes simplex virus thymidine kinase (HSVTK SEQ ID NO:42).

The suicide gene inducible amino acid sequence can be a chimeric Caspase-9 polypeptide or comprise a herpes simplex virus thymidine kinase (HSVTK SEQ ID NO:42).

The chimeric Caspase-9 polypeptide can comprise or consist of:

FKBP12 binding region comprising or consisting of a short 5′ leader peptide MLEMLE (SEQ ID NO:43) and the mutant of human FKBP12 (V36F) of sequence

GVQVETISPGDGRTFPKRGQTCVVHYTGMLEDGKKVDSSRDRN KPFKFMLGKQEVIRGWEEGVAQMSVGQRAKLTISPDYAYGATGHPGIIP PHATLVFDVELLKLE (SEQ ID NO:44) (nucleotide ID NO: BT007066.1 and Protein ID NO: AAP35729.1), which is linked by a linker, such as SGGGSG (SEQ ID NO:45) linker, to Caspase-9 polypeptide

VDGFGDVGALESLRGNADLAYILSMEPCGHCLIINNVNFCRESGL RTRTGSNIDCEKLRRRFSSLHFMVEVKGDLTAKKMVLALLELARQDHGA LDCCVVVILSHGCQASHLQFPGAVYGTDGCPVSVEKIVNIFNGTSCPSLG GKPKLFFIQACGGEQKDHGFEVASTSPEDESPGSNPEPDATPFQEGLRT FDQLDAISSLPTPSDIFVSYSTFPGFVSWRDPKSGSWYVETLDDIFEQWA HSEDLQSLLLRVANAVSVKGIYKQMPGCFNFLRKKLFFKTS (SEQ ID NO: 46) (nucleotide ID NO: AK292111.1 and Protein ID NO: BAF84800.1), which is linked by a linker, such as ASRAPR (SEQ ID NO:47) linker (containing Sacll enzyme site), to

a Polynucleotide 2A self-cleaving peptide chosen from the group consisting of T2A (derived from thosea asigna virus 2) AEGRGSLLTCGDVEENPGP (SEQ ID NO:48) (nucleotide ID NO: AF062037.1 and Protein ID NO: YP_009665206.1), P2A (derived from porcine teschovirus-1 2A) ATNFSLLKQAGDVEENPGP (SEQ ID NO: 49) (nucleotide ID NO: AB038528.1 and Protein ID NO: BAB32828.1), E2A (derived from equine rhinitis A virus) QCTNYALLKLAGDVESNPGP (SEQ ID NO:50) (nucleotide ID NO: NC_039209.1 and Protein ID NO: “YP_009513027.1”) or F2A (derived from foot-and-mouth disease virus: VKQTLNFDLLKLAGDVESNPGP (SEQ ID NO:51) (nucleotide ID NO: AY593825.1 and Protein ID NO: AAT01768.1), preferably T2A.

According to a specific embodiment of the present invention, the chimeric Caspase-9 polypeptide consists of:
the leader peptide MLEMLE (SEQ ID NO:43),
FKBP12 binding region

GVQVETISPGDGRTFPKRGQTCVVHYTGMLEDGKKVDSSRDRNK PFKFMLGKQEVIRGWEEGVAQMSVGQRAKLTISPDYAYGATGHPGIIPP HATLVFDVELLKLE (SEQ ID NO:44), which is linked by SGGGSG (SEQ ID NO:45) linker to
Caspase-9 polypeptide

VDGFGDVGALESLRGNADLAYILSMEPCGHCLIINNVNFCRESGLR TRTGSNIDCEKLRRRFSSLHFMVEVKGDLTAKKMVLALLELARQDHGAL DCCVVVILSHGCQASHLQFPGAVYGTDGCPVSVEKIVNIFNGTSCPSLG GKPKLFFIQACGGEQKDHGFEVASTSPEDESPGSNPEPDATPFQEGLRT FDQLDAISSLPTPSDIFVSYSTFPGFVSWRDPKSGSWYVETLDDIFEQWA HSEDLQSLLLRVANAVSVKGIYKQMPGCFNFLRKKLFFKTS (SEQ ID NO: 46), which is linked by a linker ASRAPR (SEQ ID NO:47) to
T2A self-cleaving peptides encoding AEGRGSLLTCGDVEENPGP (SEQ ID NO:48).

According to the present invention, the cell can be obtained in culture conditions wherein both IL-7 and IL-15 are present, for example in the culture conditions of the activation step, transduction step and/or expansion step of the process for the preparation of said cell.

The present invention concerns also a pharmaceutical composition comprising the nucleotide sequence as defined above, or the vector as defined above, or the cell as defined above, together with one or more excipients and/or adjuvants.

In addition, the present invention concerns a chimeric antigen receptor as defined above, nucleotide sequence as defined above, vector as defined above, cell as defined above, pharmaceutical composition as defined above, for medical use.

It is also an object of the present invention chimeric antigen receptor as defined above, nucleotide sequence as defined above, vector as defined above, cell as defined above, pharmaceutical composition as defined above, for use in the treatment of CD19+, CD20+ or CD22+ cancers, for example B cell lymphomas (Non-Hodgkin's Lymphoma (NHL)), acute lymphoblastic leukemia (ALL), myeloid leukemia and chronic lymphocytic leukemia (CLL).

It is also an object of the present invention chimeric antigen receptor as defined above, nucleotide sequence as defined above, vector as defined above, cell as defined above, pharmaceutical composition as defined above, for use in the treatment of autoimmune diseases caused by B cells generating auto-antibodies, including but not only limited to systemic lupus erythematosus (SLE), Systemic sclerosis (SSc), ANCA-Associated Vasculitis (AAV), Dermatomyositis (DM).

The present invention now will be described by an illustrative, but not limitative way, according to the preferred embodiments thereof, with particular reference to the examples and the enclosed drawings, wherein:

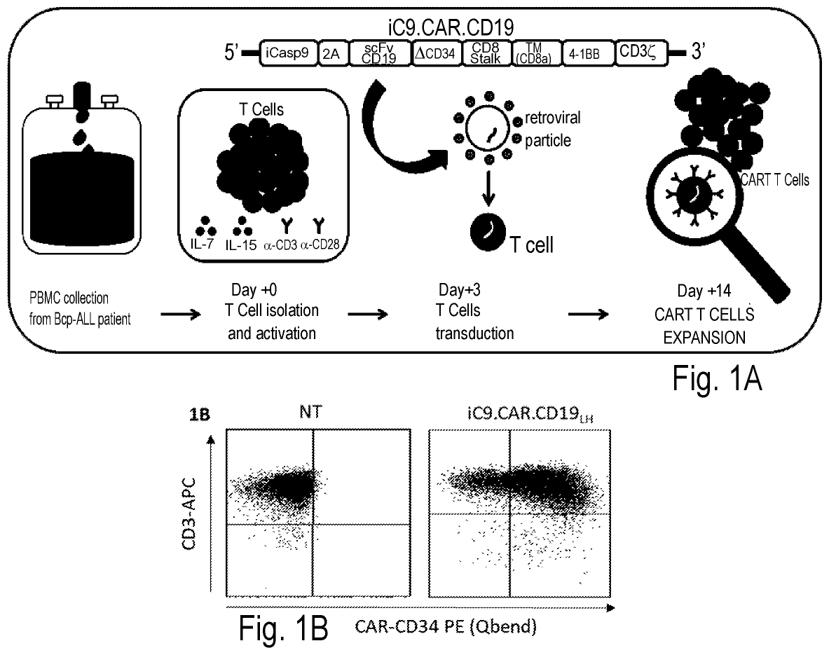

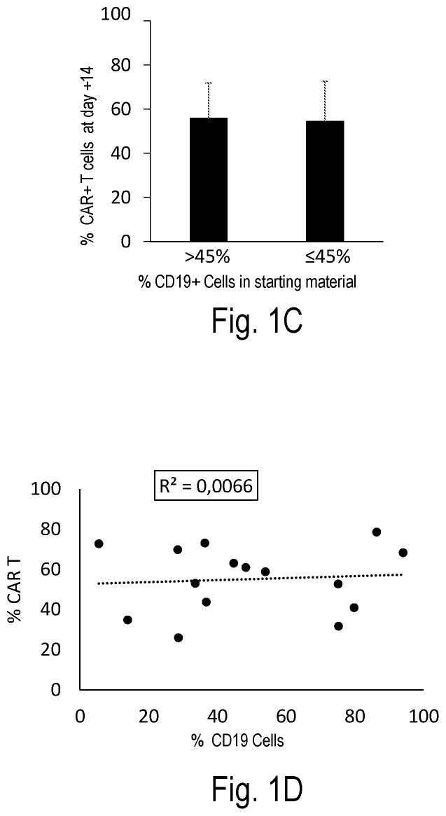

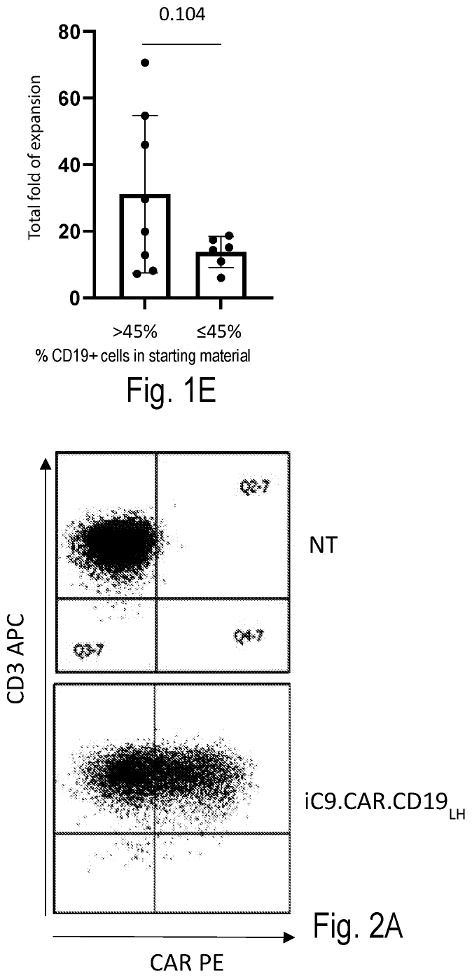

FIG. 1 shows CAR.CD19 T-cells generated from Bcp-ALL patients' derived PBMCs at diagnosis. (A) The scFv of α-CD19 is cloned in frame with the iC9 suicide gene, ΔCD34 trackable marker and both 4.1BB and the CD3ζ signaling endodomains. PBMCs of Bcp-ALL patients at diagnosis are activated with soluble α-hCD3/α-hCD28 mAbs and rh-IL7/rh-IL15 and then transduced with the CAR.CD19 γ-retroviral supernatant. (B) Flow-cytometry analysis of a representative donor showing CAR expression by ΔCD34 detection in un-transduced (NT) T-cells (negative control; left panel) and CAR.CD19 genetically modified T-cells (right panel). (C) Percentage of CAR+ T-cells at end-production (Day+14) in DPs with more than 45% (n=8) or equal to/less than 45% (n=7) of CD19+ leukemia/lymphoma cells in the starting raw material used for CAR T-cell manufacturing. The median value of 45% was used as cutoff. (D) Correlation matrix between percentage of CAR+ T-cells in the DPs at the end of production and the percentage of CD19+leukemic cells in the starting raw materials from patients. (E) Histograms representing the total fold expansion from Day +3 to the end of production of CAR T cells in the two subgroups of patients with <45% or >45% of CD19+ B cells in the SM.

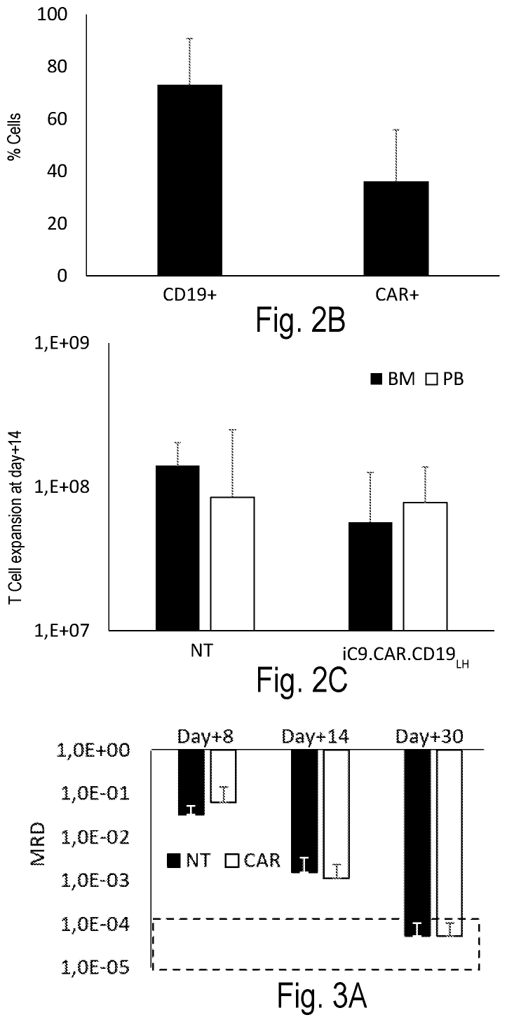

FIG. 2 shows BM Patient-derived CAR T cell proliferation and transduction. (A) Flow-cytometry analysis in a representative DP generated from BM mononuclear cells of a patient at diagnosis. Upper Panel A shows flow-cytometry analysis of CAR+ T cells in the negative control sample of NT T cells, whereas bottom panel shows the analysis in iC9.CAR.CD19 genetically modified T cells. (B) Percentage of CD19+ leukemic blasts and CAR+ T cells in DPs generated from BM Bcp-ALL patients (n=10). (C) Fold expansion of NT and iC9.CAR.CD19 BM-derived T cells (black bars) compared to PB-derived T cells (white bars) of 10 Bcp-ALL patients, at the end of production. Data are expressed as average ±SD;

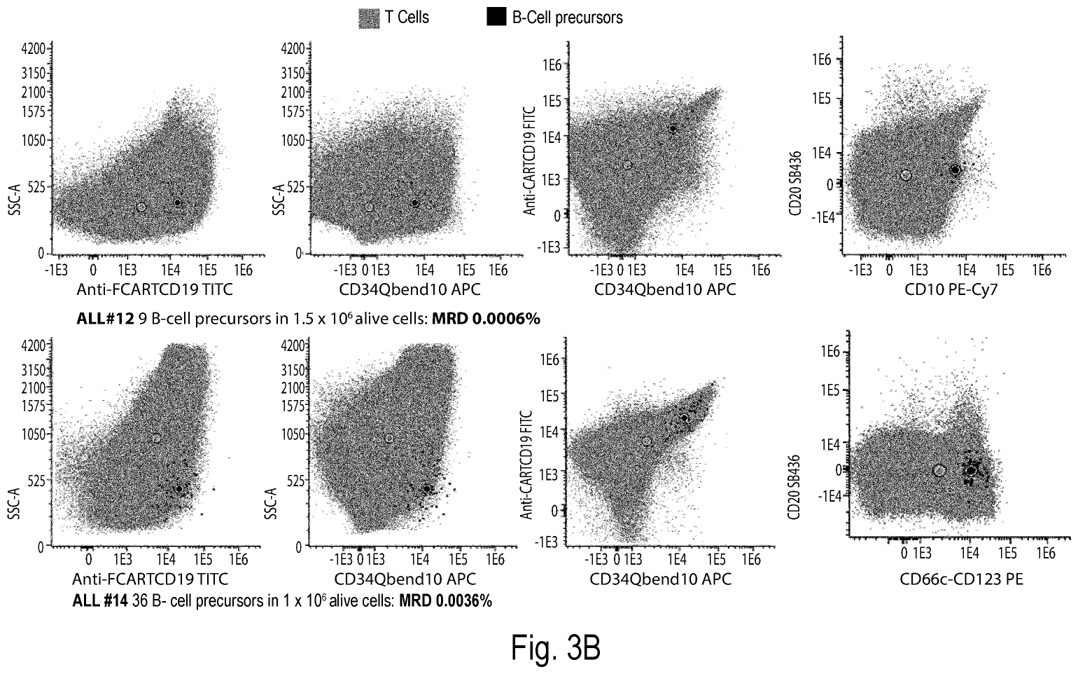

FIG. 3 shows the MRD analysis in DPs generated from row materials of patients at diagnosis highly contaminated by leukemia cells.

(A) Time-course experiments have been designed to evaluate the impact of manufacturing time on MRD value in DPs. T-cells were generated from 5 different patients (n=5) and cultured for 8, 14 or 30 days before collection of the cells for MRD analysis, performed by qPCR analysis on Ig rearrangement with detection limit specified in Table1, and represented in the figure as negative range between 10-4 and 10-5 (dotted area). (B) Flow-cytometry analysis of two DPs: ALL #12 with high MRD values and ALL #14 for which the detection of MRD was below the PCR sensitivity. Panels show the presence of leukaemia cells (black dots) positive for CAR expression, detected with both anti-CAR.CD19 FITC antibody (anti-CARTCD19, Cytognos SL, Salamanca, Spain) and anti-CD34 APC (CD34QBEND10), targeting the CD34 epitope in the CAR construct. B cell precursors were identified by the use of EuroFlow standard operating procedures (SOP) for staining of surface markers (www.EuroFlow.org) (26) by EuroFlow Bcp-ALL MRD tubes, previously described for high-sensitive MRD measurements in Bcp-ALL by flow-cytometry. (27-29)

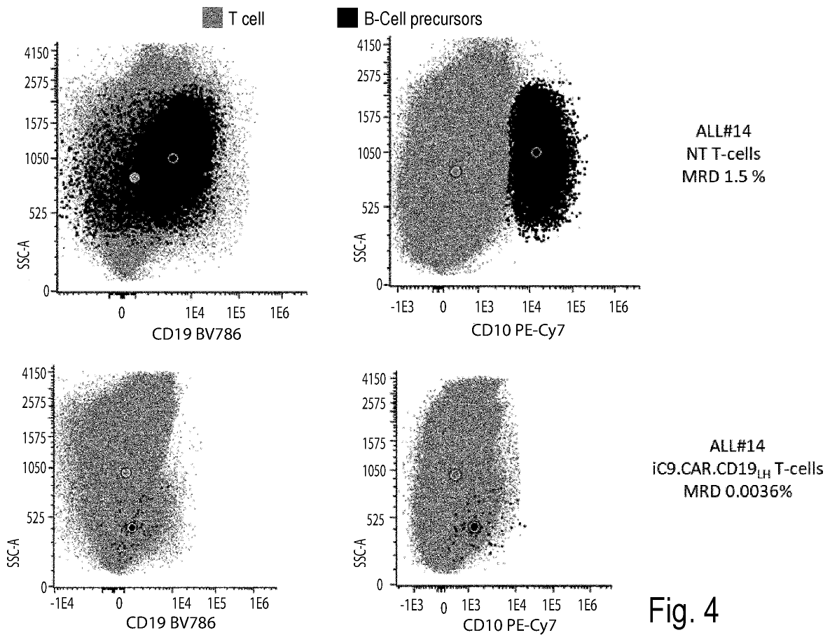

FIG. 4 shows Flow-cytometry analysis of control un-transduced T cells and iC9.CAR.CD19LH T cells from one representative Bcp-ALL patient. Upper panels show flow-cytometric analysis of CD19 and CD10 B cell markers in control un-transduced T cells from ALL#14 patient revealing 1.5% of leukemic cells, whereas the contamination was significantly reduced in the iC9.CAR.CD19LH T cell sample manufactured from the same patient ALL#14 (0.0036% of leukemic cells).

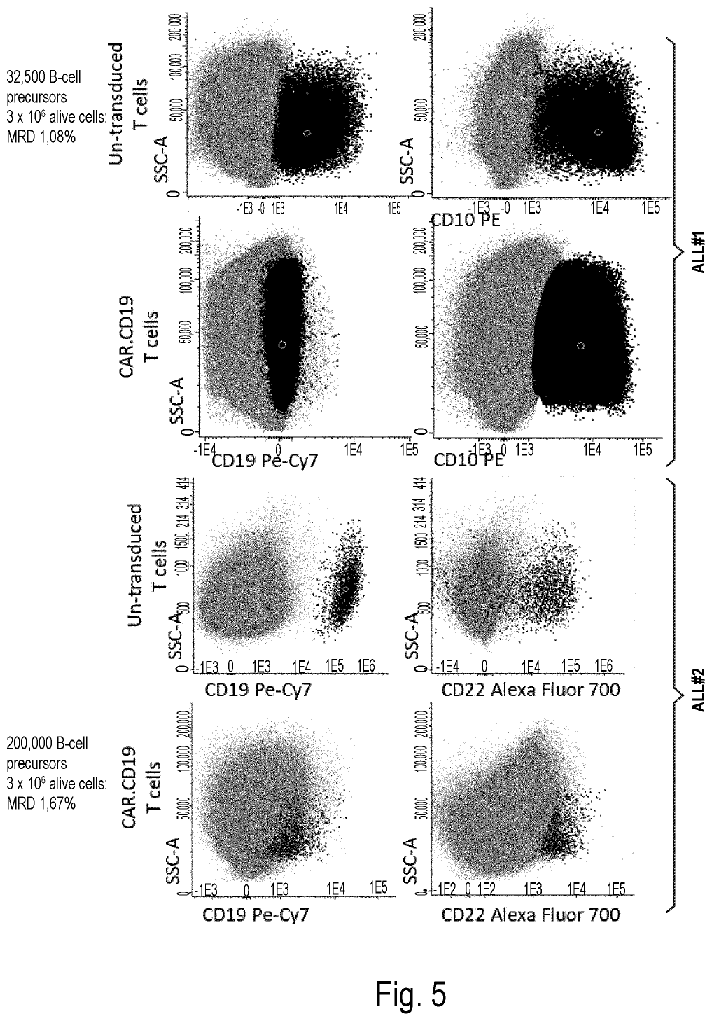

FIG. 5 shows the MRD analysis of DPs generated from BM raw materials of Bcp-ALL patients at diagnosis highly contaminated by leukaemia cells. Flow-cytometry analysis of B cell markers in two BM derived DPs from ALL#1 and ALL#2 patients. Panels show the presence of leukaemia cells (black dots);

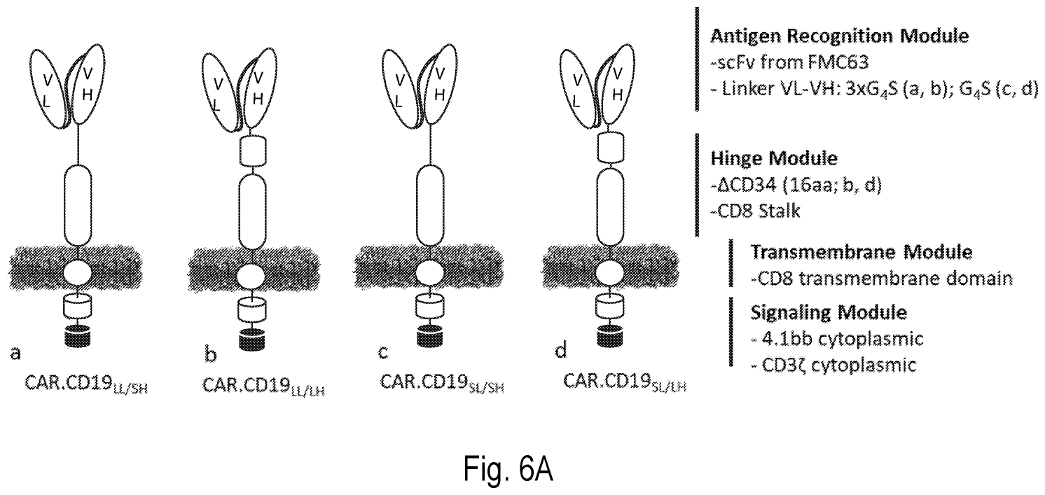

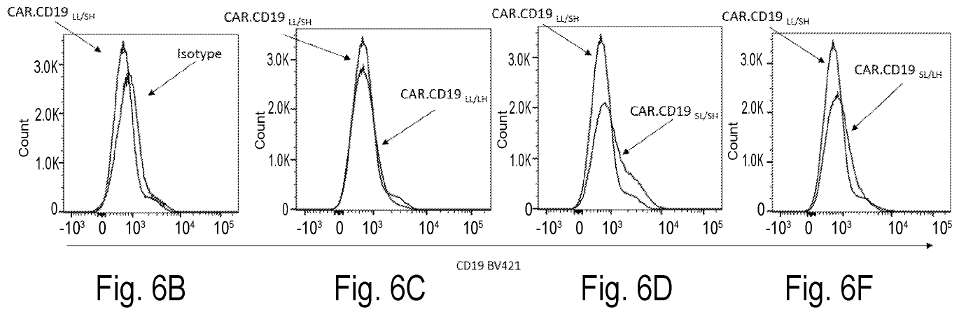

FIG. 6 shows that CAR.CD19 structure impacts on CD19 antigen engagement when they are both expressed on the same plasma membrane. (A) Cartoons representing CAR.CD19LL/SH (a), CAR.CD19LL/LH (b), CAR.CD19SL/SH (c) and CAR.CD19SL/LH (d). (B) CD19 expression detected by flow-cytometry in NALM-6 cells genetically modified by CAR.CD19LL/SH. Matched isotype staining histogram and the specific CD19 staining for CAR.CD19LL/SH cells histogram are shown (see arrows). (C-E) CD19 expression detected by flow-cytometry in NALM-6 cells genetically modified by CAR.CD19LL/LH (C), by CAR.CD19SL/SH (D) and by CAR.CD19SL/LH (E) is shown by histograms, in comparison to the histogram of CD19 expression on CAR.CD19LL/SH.

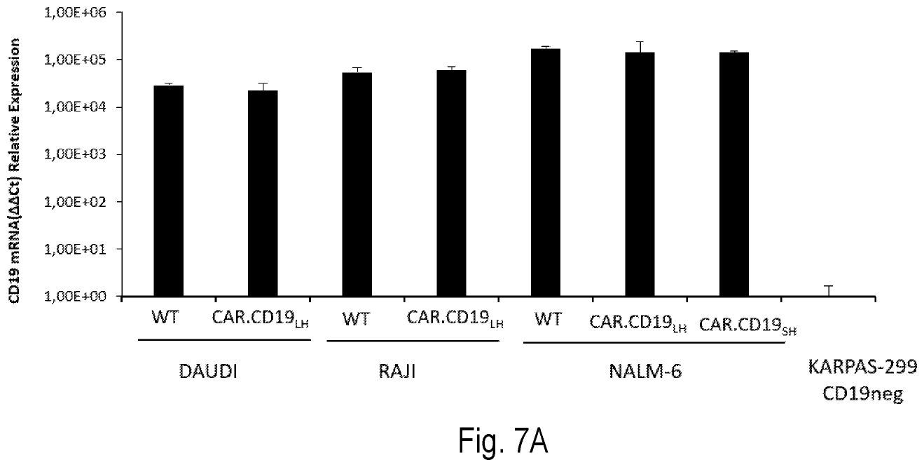

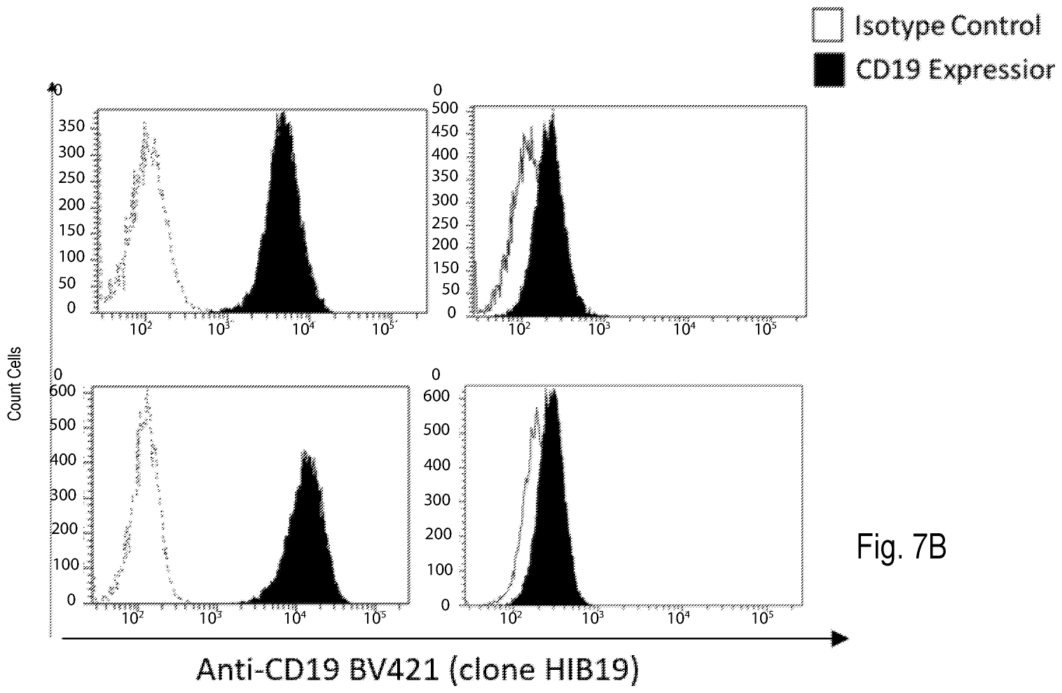

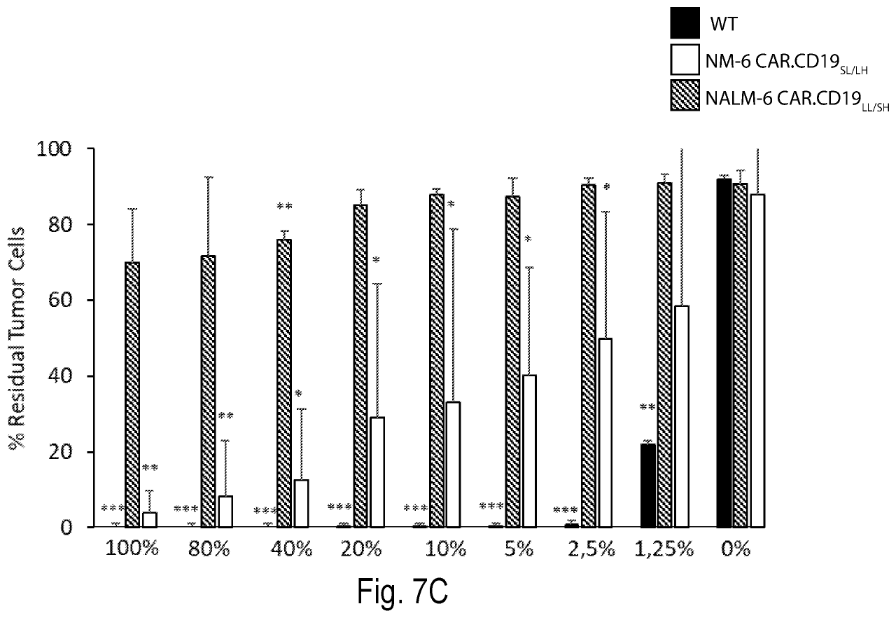

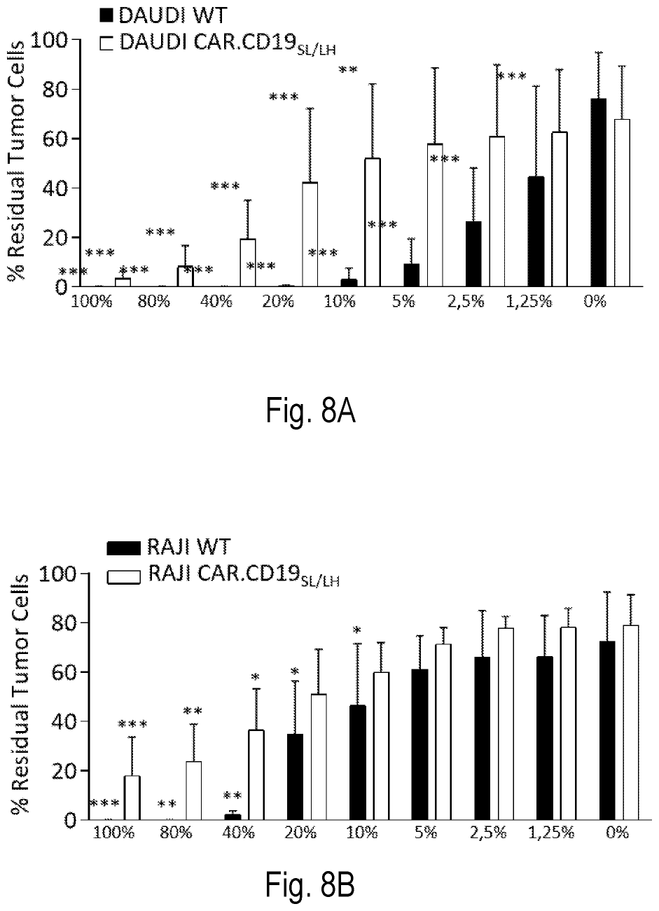

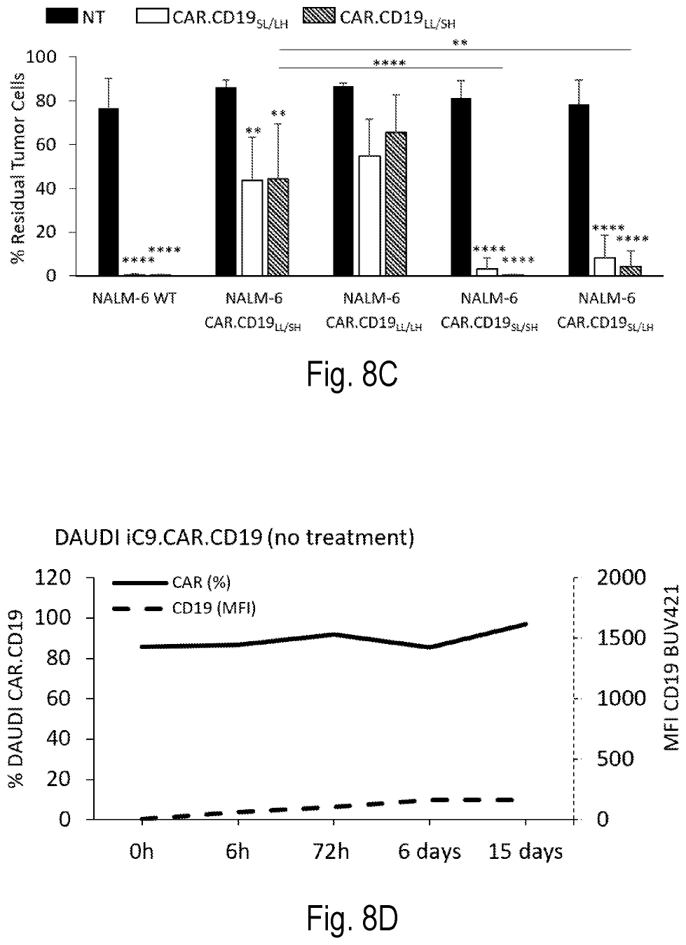

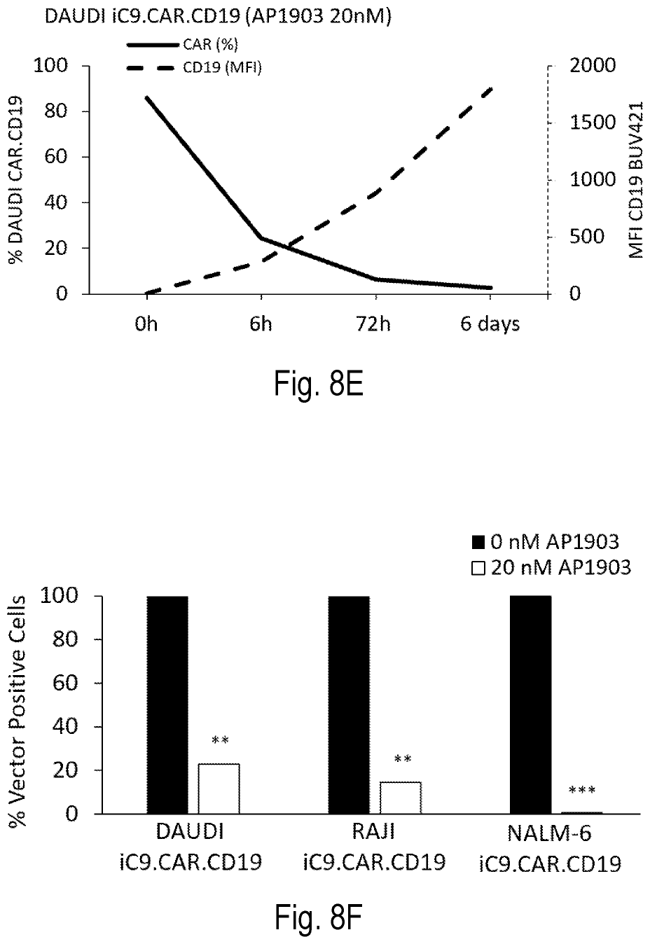

FIG. 7 shows the CD19 mRNA expression in both WT and CAR.CD19 Bcp-ALL cell lines. (A) Quantitative Real Time PCR (qRT-PCR) of CD19 mRNA expression in WT, CAR.CD19^{LH} and CAR.CD19_{SH} Bcp-ALL cell lines. Karpas cell line has been used as negative control. mRNA levels are shown as relative expression of a target gene versus ACT-B mRNA expression. Reactions were performed in triplicates; (B) MFI analysis of CAR.CD19 expression in leukaemia and lymphoma cell lines. MFI values of CAR.CD19 expression levels in WT and CAR.CD19SL/_{LH} DAUDI (top panels) and RAJI (bottom panels). Data shows one representative experiment. (C) Long-term in vitro assay to evaluate anti-tumor activity of CAR.CD19 T-cells on both WT and CAR.CD19 NALM-6 cell lines. The percentage of WT (black bars), NALM-6 CAR.CD19SL/LH (white bars) and NALM-6 CAR.CD19 UPenn (also named as CAR.CD19 LL/SH) leukemic cells (striped bars) after 7 days of in vitro co-culture. The assay was performed at decreasing effector target ratios, from 1:1 to 1:32 on NALM-6 Bcp-ALL tumor cell lines. Experiments were performed in triplicates. Data are expressed as average ±SD.*p-value=<0.05, **p-value=<0.01, ***p-value=<0.001.
FIG. 8 shows long-term in vitro assays to evaluate the activity of CAR.CD19 T-cells and iC9 controlling CAR.CD19 positive leukemia or lymphoma cell lines. (A-B) 7 days co-culture assay of WT (black bars) and CAR.CD19SL/LH (white bars) with CAR.CD19 T-cells (E:T ratio is shown in the x axis of the graph as percentage of CAR+ T-cells in the culture). (C) 7 days co-culture assay of NALM-6 WT and CAR.CD19 genetically modified NALM-6 with NT (black bars), CAR.CD19SL/LH (white bars) and CAR.CD19LL/SH (striped bars). All experiments were performed in triplicate (n=6). Data are expressed as mean±SD. *p-value=<0.05, **p-value=<0.01, ***p-value=<0.001, ****p-value=<0.0001. (D-E) CAR.CD19 DAUDI cells were treated with 0 nM (D) and 20 nM (E) AP1903; both CAR and CD19 expression were monitored over time by flow-cytometry. (F) Detection of CAR.CD19 vector in tumor cells by qRT-PCR after AP1903 exposure. Reactions were performed in triplicate. Black histograms represent the positive control of reference (OnM AP1903) and white histograms represent results after one drug exposure (20 nM of AP1903). *p-value=<0.05, **p-value=<0.01, ***p-value=<0.001.

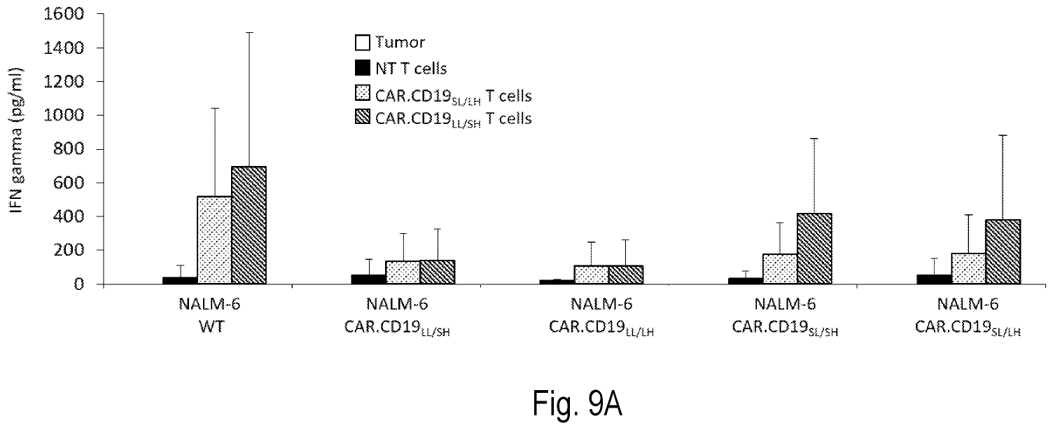

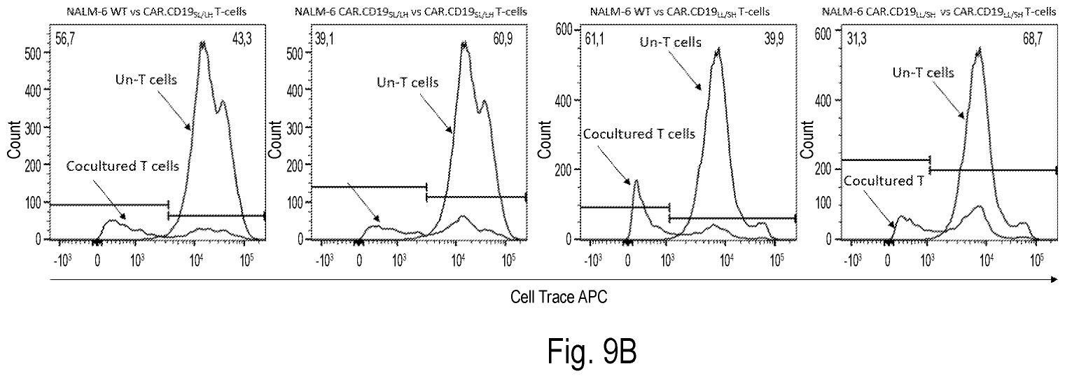

FIG. 9 shows CAR.CD19 T cells activation profile that is similar beside the CAR configuration. (A) IFN-γ production was measured after 24 h of co-culture of Effector T cells and NALM-6 WT, or NALM-6 genetically modified with CAR.CD19 constructs. Data from 6 different CAR T products generated from HDs are expressed as mean±SD. (B) CFSE Proliferation analysis representing the overlays of CAR T-cells unstimulated and stimulated with WT or CAR.CD19 modified NALM-6 cells.

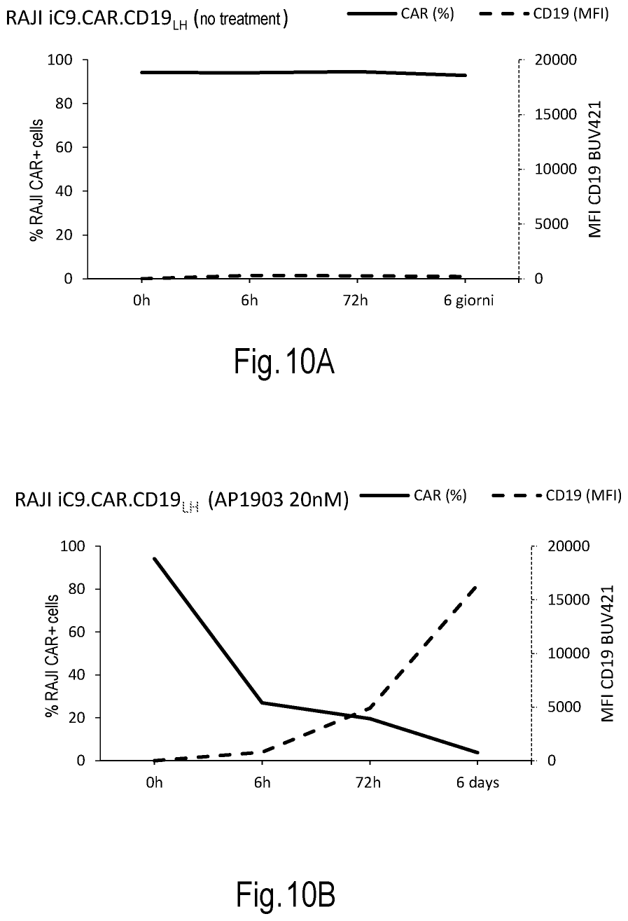

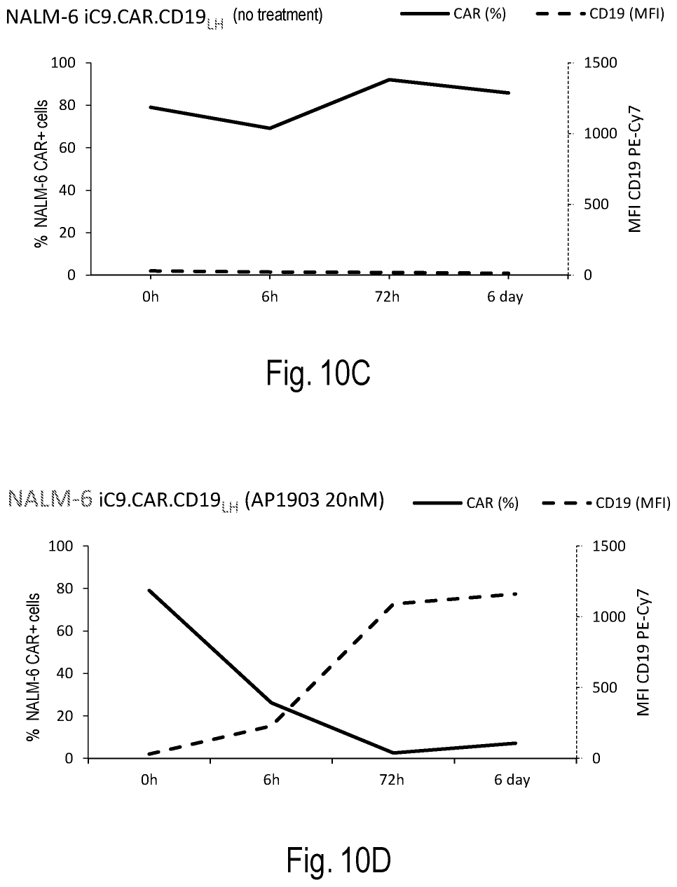

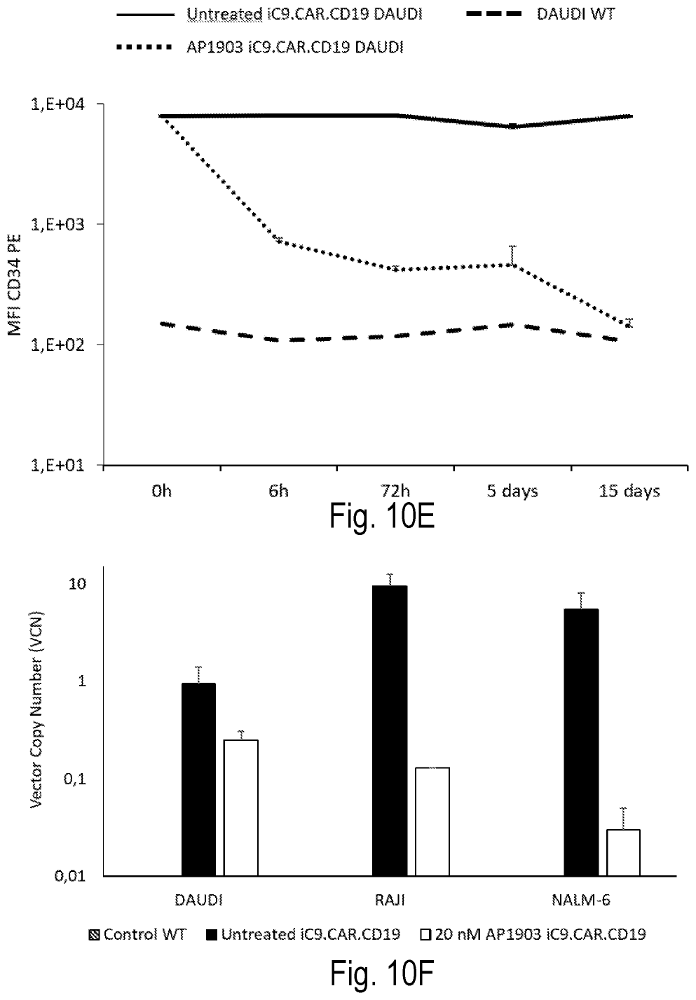

FIG. 10 shows the Effect of AP1903 administration on iC9.CAR.CD19 Bcp-ALL cell lines. iC9.CAR.CD19 (CAR.CD19LH) RAJI and NALM-6 cell lines were treated with 0 nM (A-D) and 20 nM (B-D) AP1903; both CAR and CD19 expression was monitored over time by FACS. AP1903 treatment (20 nM) results in a prompt reduction of CAR+ cells starting from 6 hours after drug exposure. The reduction of CAR positivity after AP1903 exposure is associated with a gradual detection of CD19 antigen on cell surface. (E) iC9.CAR.CD19 DAUDI cell line were treated with 0 nM (black line) and 20 nM AP1903 (short dotted line); CAR.CD19 MFI was monitored over time by flow-cytometry analysis from Day 0 to Day 15 after treatment and compared to control WT NALM-6 cell line (long dotted line). (F) Graph reporting the vector copy number (VCN) of transgene in WT and gene-modified DAUDI, RAJI and NALM-6 cell lines either untreated (black bars) or exposed to 20 nM (white bars) AP1903. Data are expressed as mean±SD.

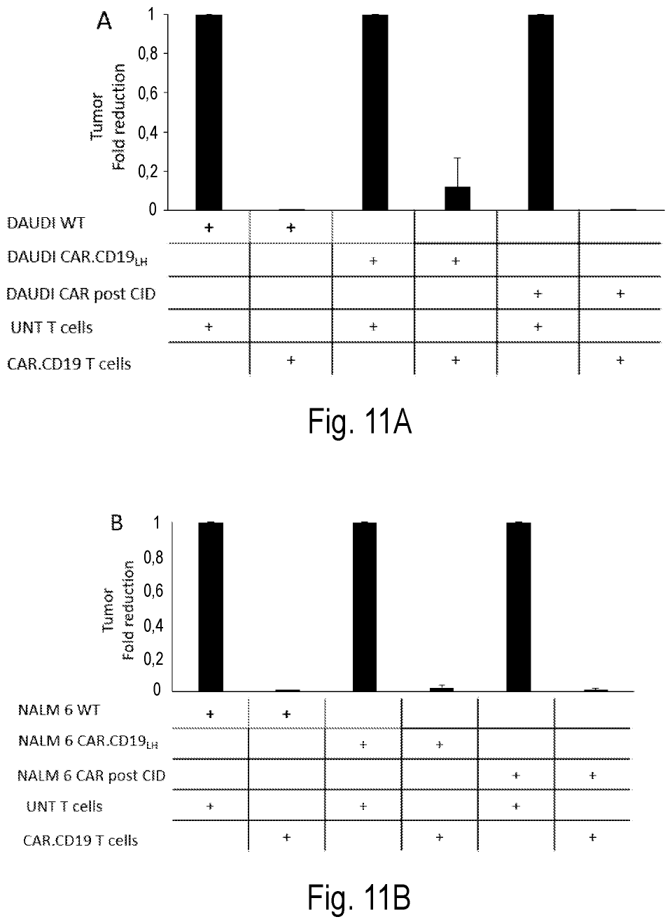

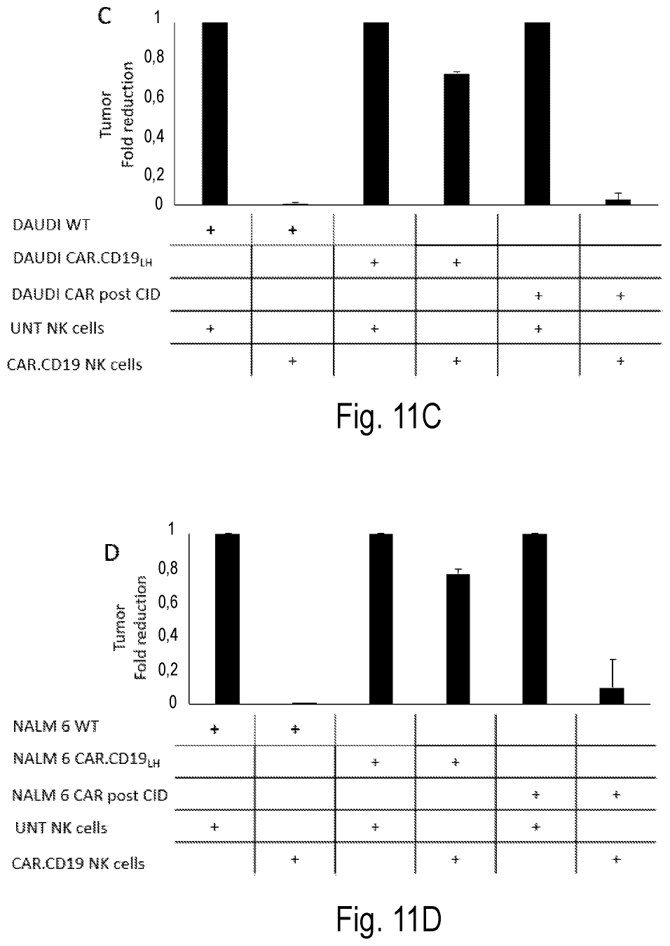

FIG. 11 shows that iC9.CAR.CD19 leukemia and lymphoma cells spared after iC9 activation could be efficiently recognized and eliminated by CAR.CD19 T cells as well as by allogenic CAR.CD19 NK cells. (A) 7-days co-culture assay was carried out between un-transduced T cells or CAR.CD19 T cells and wt DAUDI cells, iC9.CAR.CD19LH DAUDI cells never exposed to AP1903, and iC9.CAR.CD19LH DAUDI residual after AP1903 exposure and further re-expanded (at ET ratio of 1:1). (B) 7-days co-culture assay was carried out between un-transduced T cells or CAR.CD19 T cells and wt NALM-6 cells, iC9.CAR.CD19LH NALM-6 cells never exposed to AP1903, and iC9.CAR.CD19LH NALM-6 residual after AP1903 exposure and further re-expanded (at ET ratio of 1:1). (C) 7-days co-culture assay was carried out between un-transduced NK cells or CAR.CD19 NK cells and wt DAUDI cells, iC9.CAR.CD19LH DAUDI cells never exposed to AP1903, and iC9.CAR.CD19LH DAUDI residual after AP1903 exposure and further re-expanded (at E:T ratio of 1:1). (D) 7-days co-culture assay was carried out between un-transduced NK cells or CAR.CD19 NK cells and wt NALM-6 cells, iC9.CAR.CD19LH NALM-6 cells never exposed to AP1903, and iC9.CAR.CD19LH NALM-6 residual after AP1903 exposure and further re-expanded (at E:T ratio of 1:1). **p-value=<0.01, ***p-value=<0.001.

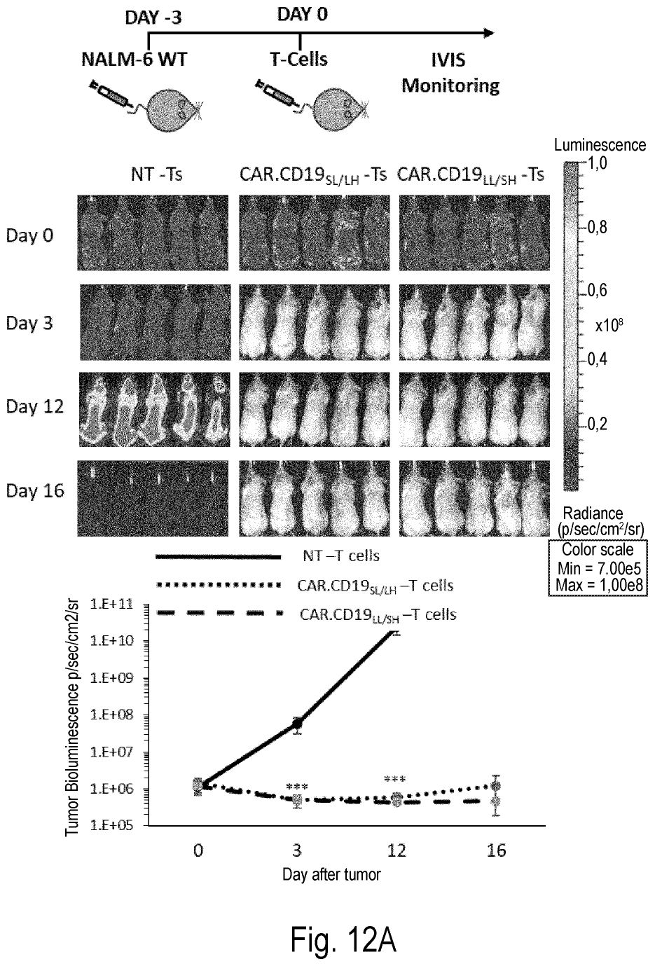

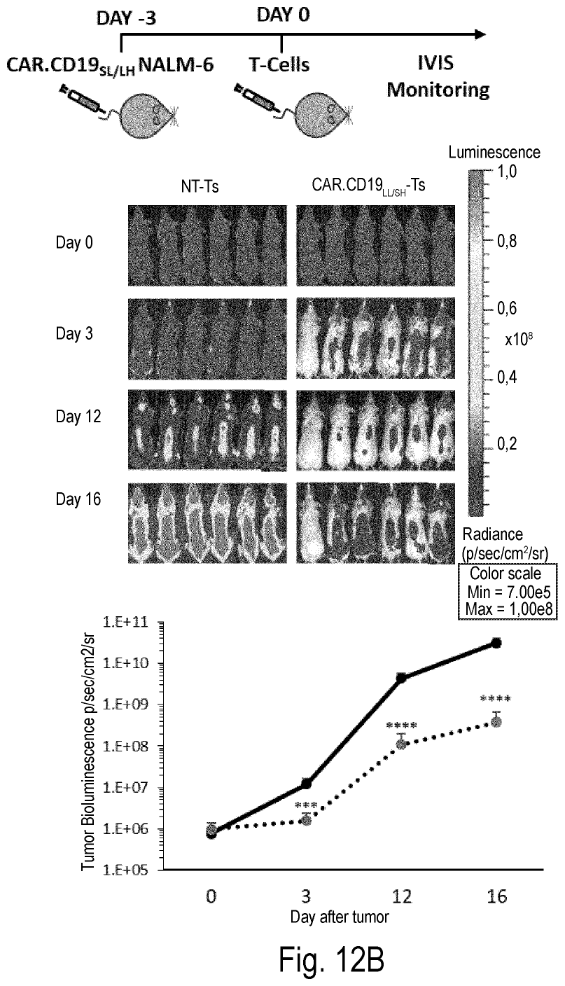

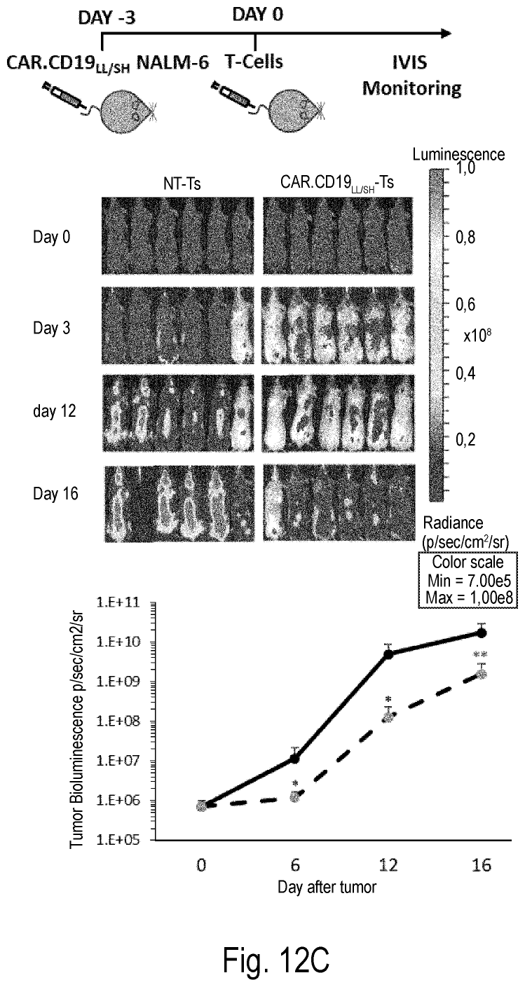

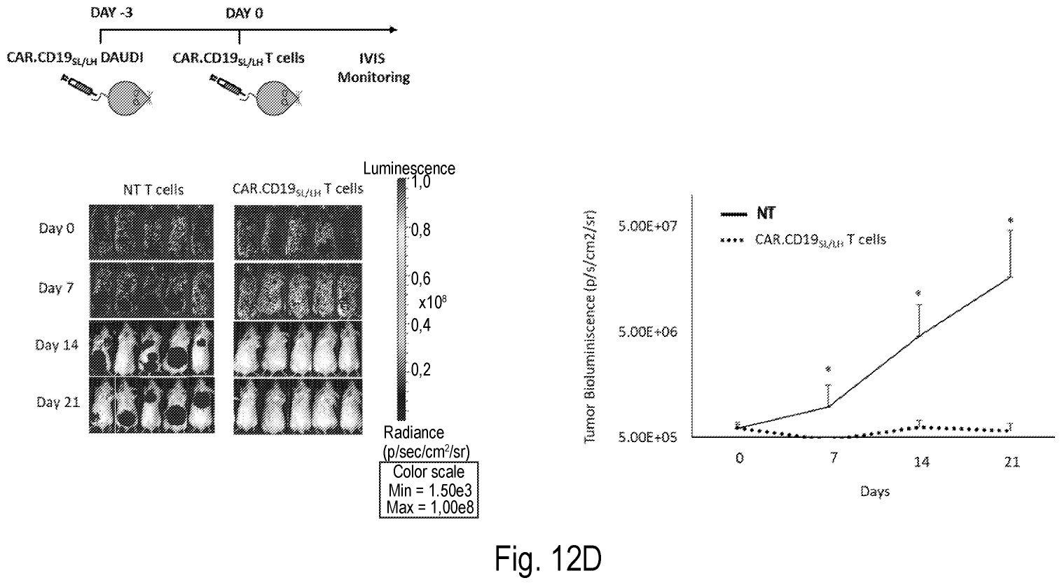

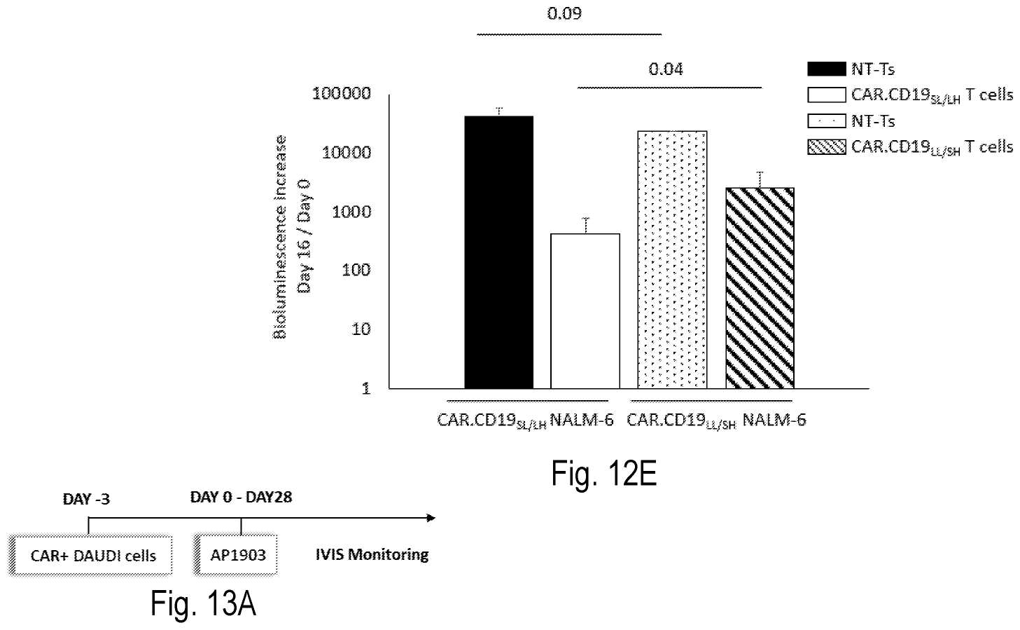

FIG. 12 shows that T-cells genetically modified with different CAR.CD19 constructs control in vivo expansion of CAR positive leukemia in a xenograft mouse model. (A) Schematic representation of the experimental design, with FF-Luciferase positive NALM-6 WT cells, infused at Day-3. At Day 0, mice were evaluated for leukemia engraftment and treated with 10×106 un-transduced (NT) or CAR.CD19SL/LH or CAR.CD19LL/SH T-cells/mouse (top panel). Bioluminescence imaging of each treated mouse (middle panel). Mean±SD of bioluminescence values of the three mice cohorts, receiving NT (black line) or CAR.CD19SL/LH T-cells (short-dotted line) or CAR.CD19LL/SH T cells (long-dotted line) (bottom panel). (B) Schematic representation of the experimental design, with CAR.CD19SL/LH positive/FF-Luciferase positive NALM-6 cells, infused at Day-3. At Day 0, mice were evaluated for leukemia engraftment and treated with 10×106 un-transduced (NT) or CAR.CD19SL/LH (top panel). Bioluminescence imaging of each treated mouse (middle panel). Mean±SD of bioluminescence values of the two mice cohorts, receiving NT (black line) or CAR.CD19SL/LH T-cells (short-dotted line) (bottom panel). (C) Schematic representation of the experimental design, with CAR.CD19LL/SH positive/FF-Luciferase positive NALM-6 cells, infused at Day-3. At Day 0, mice were evaluated for leukemia engraftment and treated with 10×106 un-transduced (NT) or CAR.CD19LL/SH (top panel). Bioluminescence imaging of each treated mouse (middle panel). Mean±SD of bioluminescence values of the two mice cohorts, receiving NT (black line) or CAR.CD19LL/SH T-cells (long-dotted line) (bottom panel). *p-value=<0.05, **p-value=<0.01, ***p-value=<0.001, ****p-value=<0.0001.

(D) Schematic representation of the experimental design, with iC9.CAR.CD19_{LH} positive FF-Luciferase positive DAUDI cells infused at day-3. At Day 0, mice were evaluated for leukemia engraftment and treated with 10×10⁶ un-transduced (NT) or CAR.CD19 T-cells/mouse. Bioluminescence images of each treated mice. Average plus standard deviation of bioluminescence values of the two mice cohorts, receiving NT (black line) or CAR.CD19 T-cells (dotted line). *p-value=<0.05. (E) Histogram representing tumor bioluminescence differences at Day 16 between mice bearing NALM-6 CAR.CD19SL/LH and CAR.CD19LL/SH NALM-6 cells. Data are shown as Mean±SD of bioluminescence increase of the two mice cohorts at Day 16 compared to Day0.

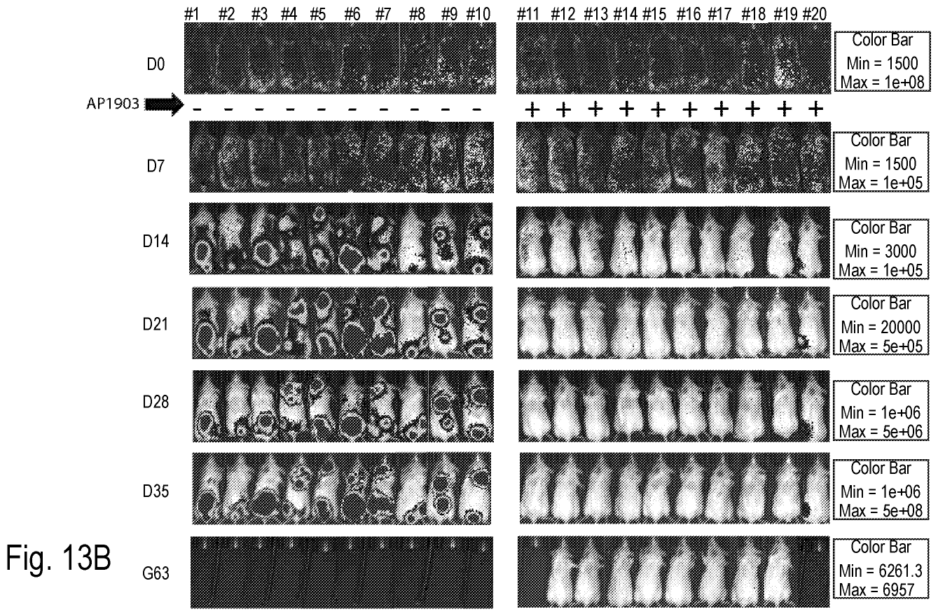

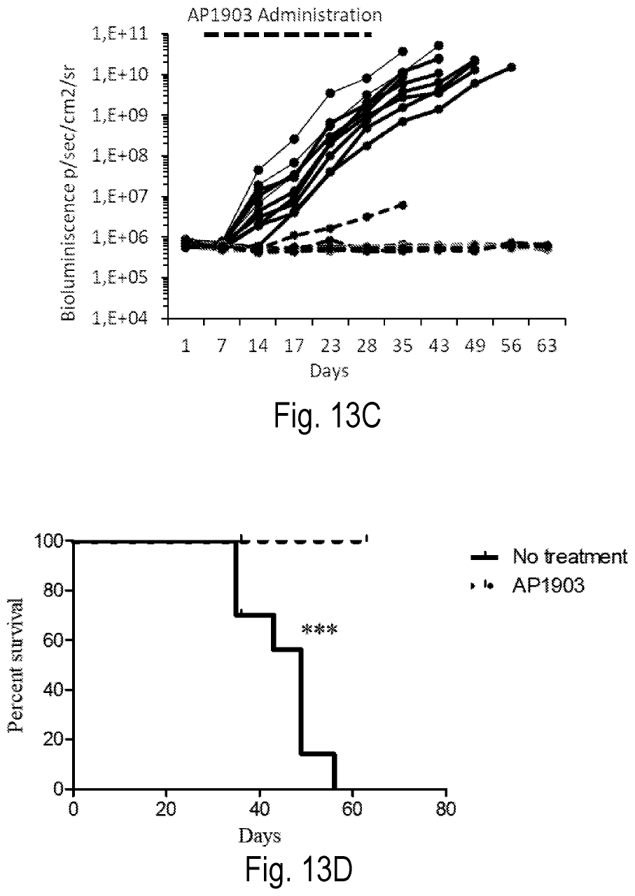

FIG. 13 shows that iC9 activation is able to control in vivo expansion of iC9.CAR positive leukemia in a xenograft mouse model. (A)

Schematic representation of the experimental design, with iC9.CAR.CD19LH positive FF-Luciferase positive DAUDI cells infused at day-3. At Day 0, mice were evaluated for leukemia engraftment and treated with 100 μg/mouse of AP1903 from day 0 to day 28. (B) Bioluminescence images of each control untreated mouse and each AP1903 treated mouse. Mice were monitored for more than 30 days after AP1903 withdrawal (C) Bioluminescence values over time for each treated mouse in the two cohorts, un-treated (black lines) or AP1903 treated (dotted line) mice. (D) Kaplan-Meier survival curve analysis of leukaemia-bearing mice untreated (black line) or AP1903 treated (blue line). ****p-value=<0.00001. The only mouse (#20) showing a persisting positive signal at IVIS analysis after AP1903 administration was sacrificed at day 35 together with a negative control (#11) and a positive control (mouse not exposed to AP1903 administration, #6), to characterize the leukemia cells.

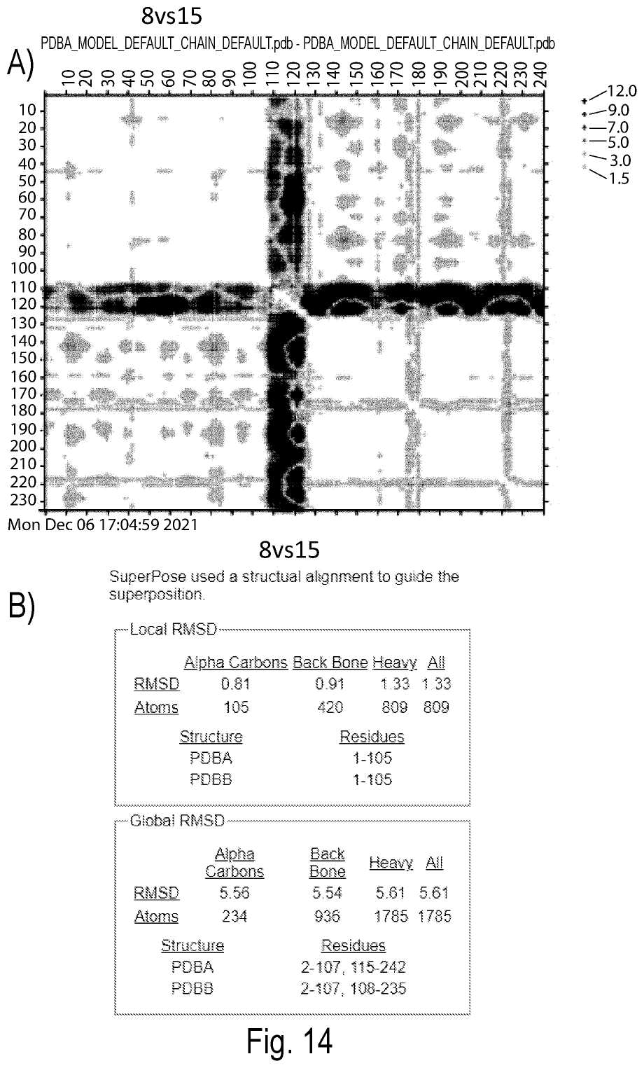

FIG. 14 shows in silico modeling data concerning CAR.CD19 15aa linker (SG4) 3 SEQ ID NO:39 vs CAR.CD19 8aa linker G3SG4 SEQ ID NO: 38.

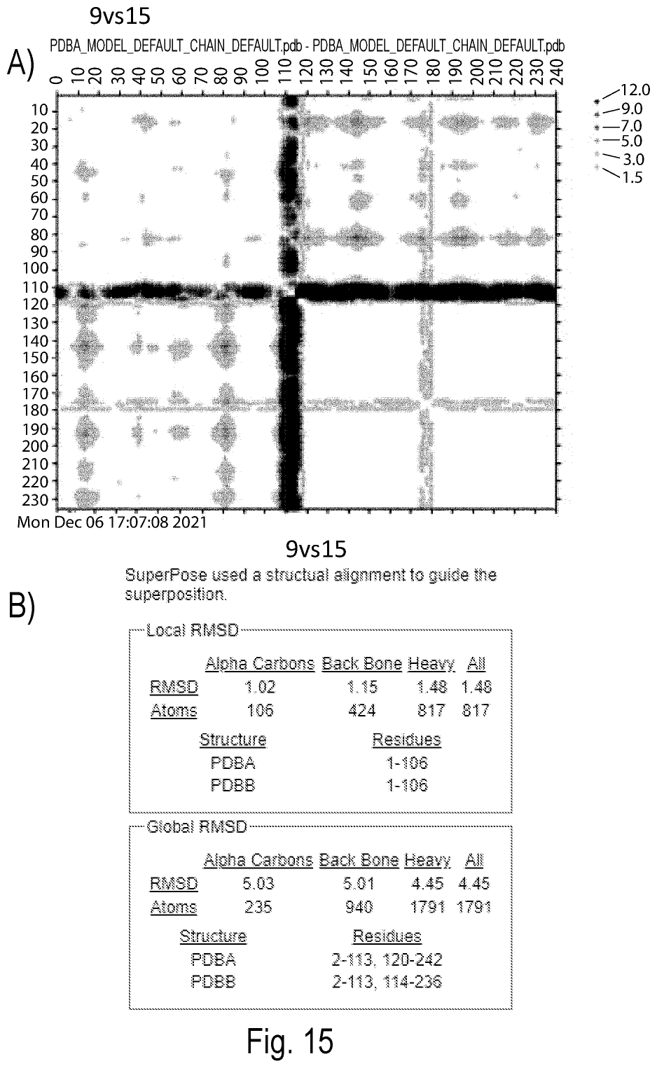

FIG. 15 shows in silico modeling data concerning CAR.CD19 15aa linker (SG4) 3 SEQ ID NO:39 vs CAR.CD19 9aa linker G4SG3 SEQ ID NO: 186.

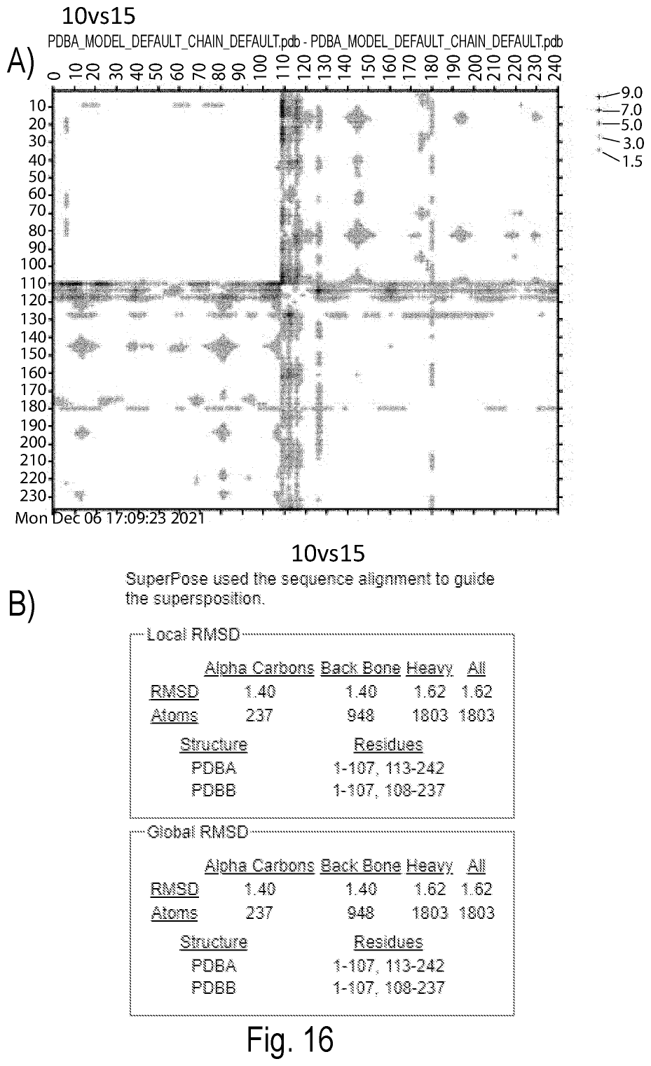

FIG. 16 shows in silico modeling data concerning CAR.CD19 15aa linker (SG4) 3 SEQ ID NO:39 vs CAR.CD19 10 aa linker (SG4)2 SEQ ID NO: 190.

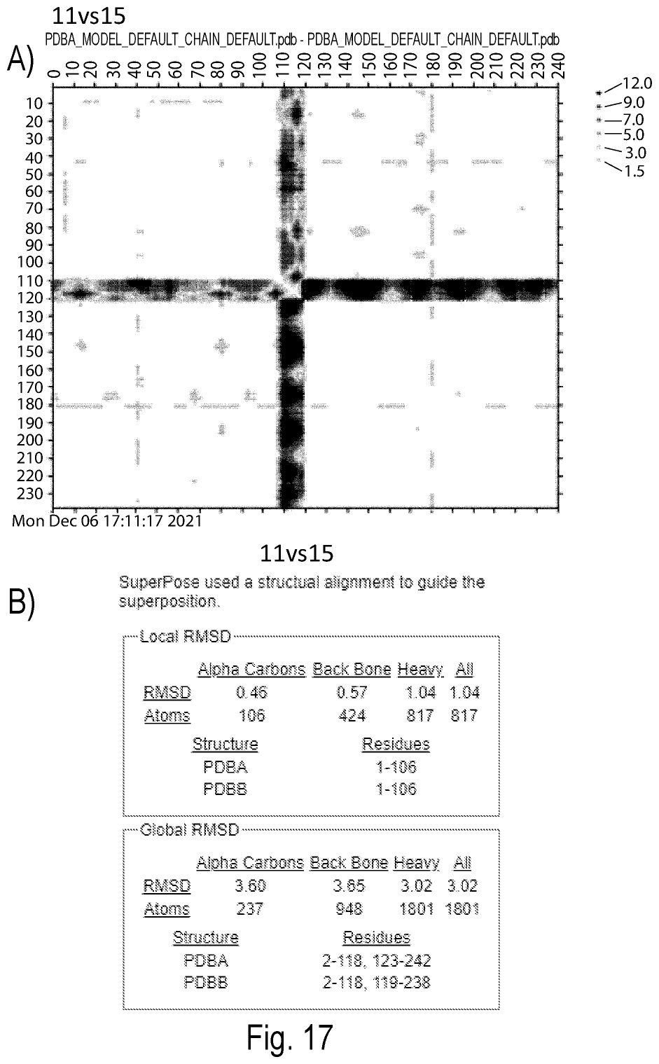

FIG. 17 shows in silico modeling data concerning CAR.CD19 15aa linker (SG4) 3 SEQ ID NO:39 vs CAR.CD19 11aa linker (SG4)2 S SEQ ID NO:187.

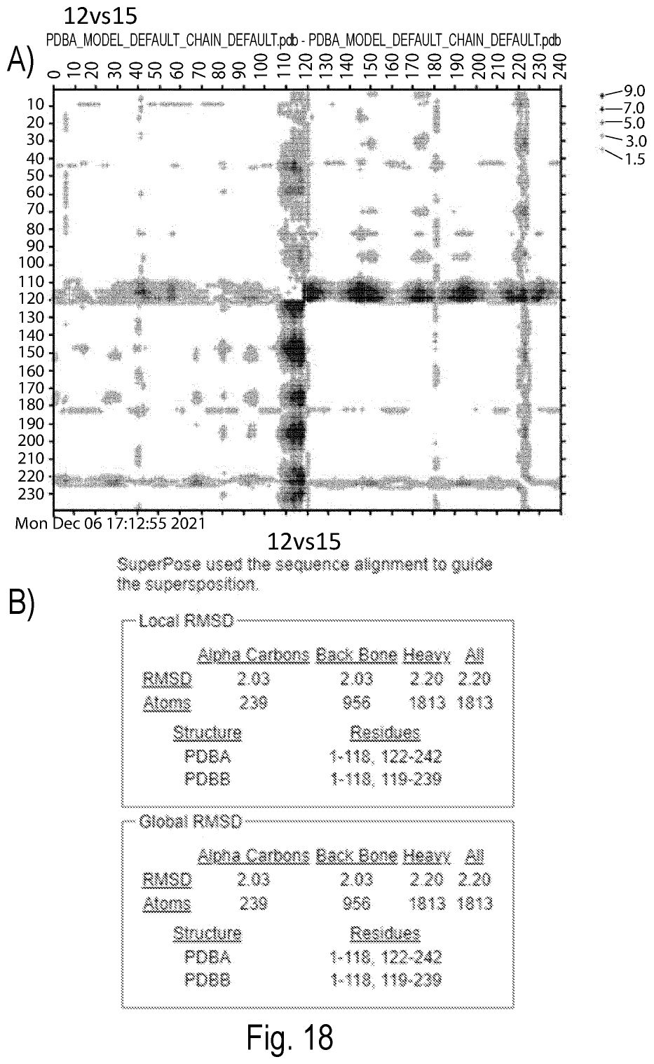

FIG. 18 shows in silico modeling data concerning CAR.CD19 15aa linker (SG4) 3 SEQ ID NO:39 vs CAR.CD19 12aa linker (SG4)2 SG SEQ ID NO:188.

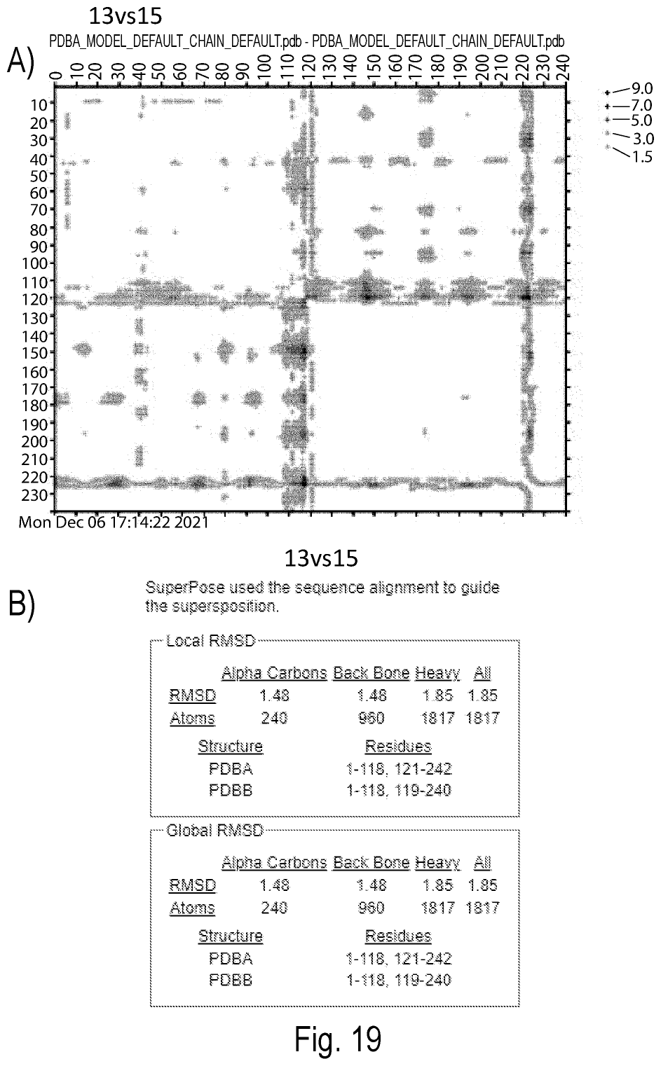

FIG. 19 shows in silico modeling data concerning CAR.CD19 15aa linker (SG4) 3 SEQ ID NO:39 vs CAR.CD19 13aa linker (SG4)2 SG2 (SEQ ID NO:191).

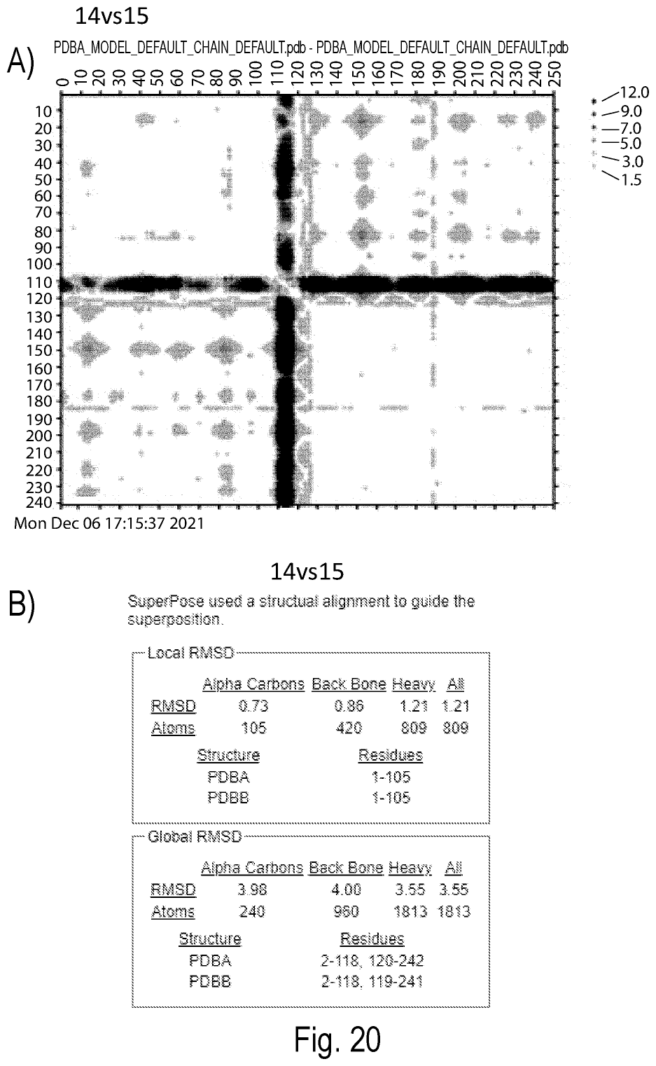

FIG. 20 shows in silico modeling data concerning CAR.CD19 15aa linker (SG4) 3 SEQ ID NO:39 vs CAR.CD19 14aa linker (SG4)2 SG3 SEQ ID NO: 189.

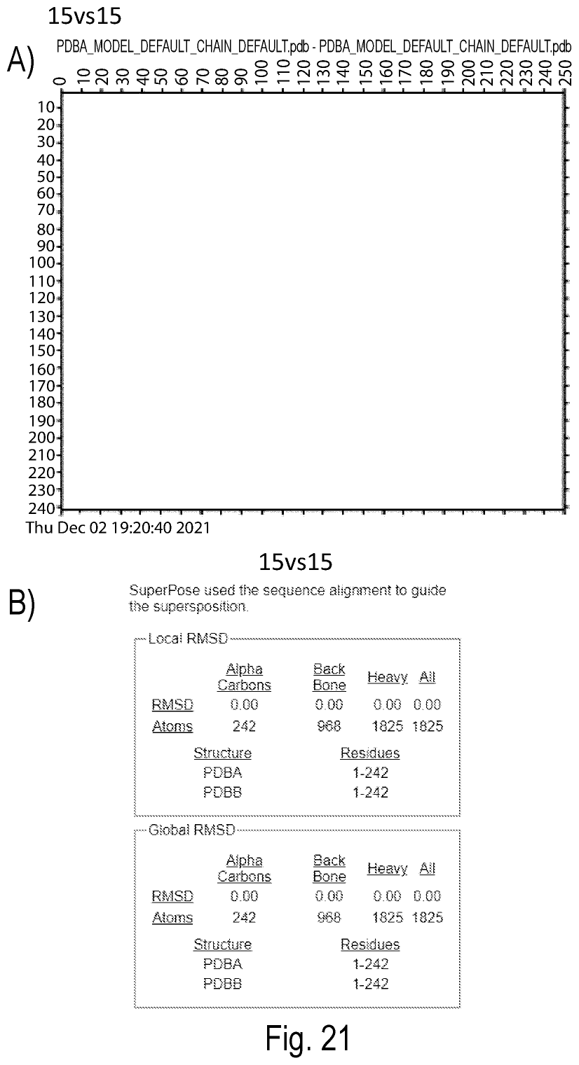

FIG. 21 shows in silico modeling data concerning CAR.CD19 15aa linker (SG4) 3 SEQ ID NO:39 vs CAR.CD19 15aa linker (SG4) 3 SEQ ID NO: 39.

### EXAMPLE 1

### CAR Vector Design According to the Present Invention And Study of the Safety Thereof in Case of Car+Leukaemia Relapse

### Materials and Methods

The biological material of human origin used in these experiments has been sampled after that both parents and healthy donors signed a written informed consent, in accordance with rules set by the Institutional Review Board (IRB) of Bambino Gesù Children's Hospital of Rome (OPBG; Approval of Ethical Committee N°969/2015 prot. N°669 LB, and N°1422/2017 prot.Nº810).

The OGMs described in the experiments were prepared in compliance with the obligations regarding OGMs, deriving from national or community regulations, and in particular from the provisions of paragraph 6 and of the legislative decrees of 12 Apr. 2001, n. 206, and 8 Jul. 2003, n. 224

### Cell Cultures

CD19 positive human Burkitt's lymphoma cell lines Daudi, NALM-6 and Raji (American Type Culture Collection Company (ATCC)), and CD19 negative Non-Hodgkin's Large Cell Lymphoma cell line Karpas-299 (Sigma Aldrich) were maintained in RPMI 1640 (EuroClone, Italy) supplemented with 10% heat-inactivated fetal bovine serum (EuroClone, Italy), 2 mM L-glutamine (GIBCO, USA), 25 IU/mL of penicillin, and 25 mg/ml of streptomycin (EuroClone, Italy), in a humidified atmosphere containing 5% CO₂ at 37° C. All cell lines were authenticated by PCR-single-locus-technology (Promega, PowerPlex 21 PCR) analysis in “BMR Genomics s.r.l.”, and were periodically checked for mycoplasma and surface markers expression.

### Effector Cells Generation and Expansion

Buffy coats (BC) from healthy donors (HDs), peripheral blood (PB) and bone marrow (BM) derived from children with Bcp-ALL were used to isolate unfractionated mononuclear cells using Lympholyte Cell Separation Media (Cedarlane, Canada). T cells were activated with soluble OKT3 and anti-CD28 (1 μg/ml, Miltenyi, Germany) monoclonal antibody (mAb) with a combination of recombinant human interleukin-7 (IL7, 10 ng/ml; R&D; USA) and interleukin-15 (IL15, 5 ng/ml; R&D; USA). NK cells were generated from BC of HDs following previously described method17. Then T and NK cells were transduced with retroviral supernatant, after three/four days, in 24-well plates pre-coated with recombinant human RetroNectin (Takara-Bio. Inc; Japan). T lymphocytes were expanded in the presence of cytokines, in TexMacs complete medium (Miltenyi, Germany) and replenished twice a week.

### CAR Constructs

Four different retroviral CAR constructs were used to carry out the experiments:

1) CAR construct carrying anti-human CD19-scFv from FMC63 clone in which VL and VH fragments were joined by a linker represented by three GSSSS repetitions (3xG4S, long linker, LL), in frame with CD8 stalk domain (short hinge, SH), CD8 transmembrane domain, 4.1bb and CD3ζ cytoplasmic domain (CAR.CD19 VL-3GS-VH-CD8-4.1bb.ζ, i.e. LL/SH);

CAR.CD19 LL/SH nt
(SEQ ID NO: 73)
ATGGAGTTTGGACTTTCTTGGTTGTTTTTGGTGGCAATTCTGAAGGGTGT
CCAGTGTAGCAGGGACATCCAGATGACACAGACTACATCCTCCCTGTCTG
CCTCTCTGGGAGACAGAGTCACCATCAGTTGCAGGGCAAGTCAGGACATT
AGTAAATATTTAAATTGGTATCAGCAGAAACCAGATGGAACTGTTAAACT
CCTGATCTACCATACATCAAGATTACACTCAGGAGTCCCATCAAGGTTCA
GTGGCAGTGGGTCTGGAACAGATTATTCTCTCACCATTAGCAACCTGGAG
CAAGAAGATATTGCCACTTACTTTTGCCAACAGGGTAATACGCTTCCGTA
CACGTTCGGAGGGGGGACTAAGTTGGAAATAACAAGCGGAGGTGGGGGCA
GCGGAGGTGGGGGCAGCGGAGGTGGGGGCGAGGTGAAACTGCAGGAGTCA
GGACCTGGCCTGGTGGCGCCCTCACAGAGCCTGTCCGTCACATGCACTGT
CTCAGGGGTCTCATTACCCGACTATGGTGTAAGCTGGATTCGCCAGCCTC
CACGAAAGGGTCTGGAGTGGCTGGGAGTAATATGGGGTAGTGAAACCACA
TACTATAATTCAGCTCTCAAATCCAGACTGACCATCATCAAGGACAACTC
CAAGAGCCAAGTTTTCTTAAAAATGAACAGTCTGCAAACTGATGACACAG
CCATTTACTACTGTGCCAAACATTATTACTACGGTGGTAGCTATGCTATG
GACTACTGGGGTCAAGGAACCTCAGTCACCGTCTCCTCAGGATCCCCCGC
CCCAAGACCCCCCACACCTGCGCCGACCATTGCTTCTCAACCCCTGAGTT
TGAGACCCGAGGCCTGCCGGCCAGCTGCCGGCGGGGCCGTGCATACAAGA
GGACTCGATTTCGCTTGCGACATCTATATCTGGGCACCTCTCGCTGGCAC
CTGTGGAGTCCTTCTGCTCAGCCTGGTTATTACTCTGTACTGTAATCACC
GGAATCGCCGCCGCGTTTGTAAGTGTCCCAGGGTCGACAAACGGGGCAGA
AAGAAACTCCTGTATATATTCAAACAACCATTTATGAGACCAGTACAAAC
TACTCAAGAGGAAGATGGCTGTAGCTGCCGATTTCCAGAAGAAGAAGAAG
GAGGATGTGAACTGAGAGTGAAGTTCAGCAGGAGCGCAGACGCCCCCGCG
TACCAGCAGGGCCAGAACCAGCTCTATAACGAGCTCAATCTAGGACGAAG
AGAGGAGTACGATGTTTTGGACAAGAGACGTGGCCGGGACCCTGAGATGG
GGGGAAAGCCGAGAAGGAAGAACCCTCAGGAAGGCCTGTACAATGAACTG
CAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAG
CCACCAAGGACACCTACGACGCCCTTCACATGCAGGCCCTGCCCCCTCGC
TAA
CAR.CD19 LL/SH aa
(SEQ ID NO: 74)
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITSGGGGSGGGGGGGGEVKLQESG
PGLVAPSQSLSVTCTVSGVSLPDYGVSWIRQPPRKGLEWLGVIWGSETTY
YNSALKSRLTIIKDNSKSQVFLKMNSLQTDDTAIYYCAKHYYYGGSYAMD
YWGQGTSVTVSSGSPAPRPPTPAPTIASQPLSLRPEACRPAAGGAVHTRG
LDFACDIYIWAPLAGTCGVLLLSLVITLYCNHRNRRRVCKCPRVDKRGRK
KLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR-

2) CAR construct carrying anti-human CD19-scFv from FMC63 clone in which VL and VH fragments were joined by a linker represented by three GSSSS repetitions (3xG4S, long linker, LL), in frame with 16aa sequence derived from human CD34 antigen (ΔCD34, long hinge, LH), CD8 stalk domain, CD8 transmembrane domain, 4.1bb and CD3ζ cytoplasmic domain (CAR.CD19 VL-3GS-VH-CD34-CD8-4.1bb.ζ, i.e. LL/LH);

CAR.CD19LL/LH nt sequence
(SEQ ID NO: 36):
ATGGAGTTTGGACTTTCTTGGTTGTTTTTGGTGGCAATTCTGAAGGGTGT
CCAGTGTAGCAGGGACATCCAGATGACACAGACTACATCCTCCCTGTCTG
CCTCTCTGGGAGACAGAGTCACCATCAGTTGCAGGGCAAGTCAGGACATT
AGTAAATATTTAAATTGGTATCAGCAGAAACCAGATGGAACTGTTAAACT
CCTGATCTACCATACATCAAGATTACACTCAGGAGTCCCATCAAGGTTCA
GTGGCAGTGGGTCTGGAACAGATTATTCTCTCACCATTAGCAACCTGGAG
CAAGAAGATATTGCCACTTACTTTTGCCAACAGGGTAATACGCTTCCGTA
CACGTTCGGAGGGGGGACTAAGTTGGAAATAACAAGCGGAGGTGGGGGCA
GCGGAGGTGGGGGCAGCGGAGGTGGGGGCGAGGTGAAACTGCAGGAGTCA
GGACCTGGCCTGGTGGCGCCCTCACAGAGCCTGTCCGTCACATGCACTGT
CTCAGGGGTCTCATTACCCGACTATGGTGTAAGCTGGATTCGCCAGCCTC
CACGAAAGGGTCTGGAGTGGCTGGGAGTAATATGGGGTAGTGAAACCACA
TACTATAATTCAGCTCTCAAATCCAGACTGACCATCATCAAGGACAACTC
CAAGAGCCAAGTTTTCTTAAAAATGAACAGTCTGCAAACTGATGACACAG
CCATTTACTACTGTGCCAAACATTATTACTACGGTGGTAGCTATGCTATG
GACTACTGGGGTCAAGGAACCTCAGTCACCGTCTCCTCAGGATCCGCATG
CGAACTTCCTACTCAGGGGACTTTCTCAAACGTTAGCACAAACGTAAGTG
CGGCCGCcCCCGCCCCAAGACCCCCCACACCTGCGCCGACCATTGCTTCT
CAACCCCTGAGTTTGAGACCCGAGGCCTGCCGGCCAGCTGCCGGCGGGGC
CGTGCATACAAGAGGACTCGATTTCGCTTGCGACATCTATATCTGGGCAC
CTCTCGCTGGCACCTGTGGAGTCCTTCTGCTCAGCCTGGTTATTACTCTG
TACTGTAATCACCGGAATCGCCGCCGCGTTTGTAAGTGTCCCAGGGTCGA
CAAACGGGGCAGAAAGAAACTCCTGTATATATTCAAACAACCATTTATGA
GACCAGTACAAACTACTCAAGAGGAAGATGGCTGTAGCTGCCGATTTCCA
GAAGAAGAAGAAGGAGGATGTGAACTGAGAGTGAAGTTCAGCAGGAGCGC
AGACGCCCCCGCGTACCAGCAGGGCCAGAACCAGCTCTATAACGAGCTCA
ATCTAGGACGAAGAGAGGAGTACGATGTTTTGGACAAGAGACGTGGCCGG
GACCCTGAGATGGGGGGAAAGCCGAGAAGGAAGAACCCTCAGGAAGGCCT
GTACAATGAACTGCAGAAAGATAAGATGGCGGAGGCCTACAGTGAGATTG
GGATGAAAGGCGAGCGCCGGAGGGGCAAGGGGCACGATGGCCTTTACCAG
GGTCTCAGTACAGCCACCAAGGACACCTACGACGCCCTTCACATGCAGGC
CCTGCCCCCTCGCTAA
CAR.CD19LL/LH aa sequence
(SEQ ID NO: 39):
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITSGGGGSGGGGSGGGGEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSGSACELPTQGTFSNVSTNVSAAAPAPRPPTPAPTIAS
QPLSLRPEACRPAAGGAVHTRGLDFACDIYIWAPLAGTCGVLLLSLVITL
YCNHRNRRRVCKCPRVDKRGRKKLLYIFKQPFMRPVQTTQEEDGCSCRFP
EEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPR-

3) CAR construct carrying anti-human CD19-scFv from FMC63 clone in which VL and VH fragments were joined by a linker represented by one GGGSGGGG repetition (SEQ ID NO:38) (G3SG4, short linker, SL), in frame with CD8 stalk domain (short hinge, SH), CD8 transmembrane domain, 4.1bb and CD3ζ cytoplasmic domain (CAR.CD19 VL-1GS-VH-CD8-4.1bb.ζ, i.e. SL/SH);

CAR.CD19 SL/SH nt sequence
(SEQ ID NO: 34):
ATGGAGTTTGGACTTTCTTGGTTGTTTTTGGTGGCAATTCTGAAGGGTGT
CCAGTGTAGCAGGGACATCCAGATGACACAGACTACATCCTCCCTGTCTG
CCTCTCTGGGAGACAGAGTCACCATCAGTTGCAGGGCAAGTCAGGACATT
AGTAAATATTTAAATTGGTATCAGCAGAAACCAGATGGAACTGTTAAACT
CCTGATCTACCATACATCAAGATTACACTCAGGAGTCCCATCAAGGTTCA
GTGGCAGTGGGTCTGGAACAGATTATTCTCTCACCATTAGCAACCTGGAG
CAAGAAGATATTGCCACTTACTTTTGCCAACAGGGTAATACGCTTCCGTA
CACGTTCGGAGGGGGGACTAAGTTGGAAATAACAGGCGGAGGAAGCGGAG
GTGGGGGCGAGGTGAAACTGCAGGAGTCAGGACCTGGCCTGGTGGCGCCC
TCACAGAGCCTGTCCGTCACATGCACTGTCTCAGGGGTCTCATTACCCGA
CTATGGTGTAAGCTGGATTCGCCAGCCTCCACGAAAGGGTCTGGAGTGGC
TGGGAGTAATATGGGGTAGTGAAACCACATACTATAATTCAGCTCTCAAA
TCCAGACTGACCATCATCAAGGACAACTCCAAGAGCCAAGTTTTCTTAAA
AATGAACAGTCTGCAAACTGATGACACAGCCATTTACTACTGTGCCAAAC
ATTATTACTACGGTGGTAGCTATGCTATGGACTACTGGGGTCAAGGAACC
TCAGTCACCGTCTCCTCAGGATCCCCCGCCCCAAGACCCCCCACACCTGC
GCCGACCATTGCTTCTCAACCCCTGAGTTTGAGACCCGAGGCCTGCCGGC
CAGCTGCCGGCGGGGCCGTGCATACAAGAGGACTCGATTTCGCTTGCGAC
ATCTATATCTGGGCACCTCTCGCTGGCACCTGTGGAGTCCTTCTGCTCAG
CCTGGTTATTACTCTGTACTGTAATCACCGGAATCGCCGCCGCGTTTGTA
AGTGTCCCAGGGTCGACAAACGGGGCAGAAAGAAACTCCTGTATATATTC
AAACAACCATTTATGAGACCAGTACAAACTACTCAAGAGGAAGATGGCTG
TAGCTGCCGATTTCCAGAAGAAGAAGAAGGAGGATGTGAACTGAGAGTGA
AGTTCAGCAGGAGCGCAGACGCCCCCGCGTACCAGCAGGGCCAGAACCAG
CTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGA
ACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCA
CGATGGCCTTTACCAGGGTCTCAGTACAGCCACCAAGGACACCTACGACG
CCCTTCACATGCAGGCCCTGCCCCCTCGCTAAA
CAR.CD19 SL/SH aa sequence
(SEQ ID NO: 33):
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGSGGGGEVKLQESGPGLVAP
SQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGT
SVTVSSGSPAPRPPTPAPTIASQPLSLRPEACRPAAGGAVHTRGLDFACD
IYIWAPLAGTCGVLLLSLVITLYCNHRNRRRVCKCPRVDKRGRKKLLYIF
KQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPR

4) CAR construct carrying anti-human CD19-scFv from FMC63 clone in which VL and VH fragments were joined by a linker represented by one GGGSGGGG (SEQ ID NO:38) repetition (G3SG4, short linker, SL), in frame with 16aa sequence derived from human CD34 antigen (ΔCD34, long hinge, LH), CD8 stalk domain, CD8 transmembrane domain, 4.1bb and CD3ζ cytoplasmic domain (CAR.CD19 VL-1GS-VH-CD34-CD8-4.1bb.ζ, i.e. SL/LH).

CAR.CD19SL/LH nt sequence
(SEQ ID NO: 58):
ATGGAGTTTGGACTTTCTTGGTTGTTTTTGGTGGCAATTCTGAAGGGTGT
CCAGTGTAGCAGGGACATCCAGATGACACAGACTACATCCTCCCTGTCTG
CCTCTCTGGGAGACAGAGTCACCATCAGTTGCAGGGCAAGTCAGGACATT
AGTAAATATTTAAATTGGTATCAGCAGAAACCAGATGGAACTGTTAAACT
CCTGATCTACCATACATCAAGATTACACTCAGGAGTCCCATCAAGGTTCA
GTGGCAGTGGGTCTGGAACAGATTATTCTCTCACCATTAGCAACCTGGAG
CAAGAAGATATTGCCACTTACTTTTGCCAACAGGGTAATACGCTTCCGTA
CACGTTCGGAGGGGGGACTAAGTTGGAAATAACAGGCGGAGGAAGCGGAG
GTGGGGGCGAGGTGAAACTGCAGGAGTCAGGACCTGGCCTGGTGGCGCCC
TCACAGAGCCTGTCCGTCACATGCACTGTCTCAGGGGTCTCATTACCCGA
CTATGGTGTAAGCTGGATTCGCCAGCCTCCACGAAAGGGTCTGGAGTGGC
TGGGAGTAATATGGGGTAGTGAAACCACATACTATAATTCAGCTCTCAAA
TCCAGACTGACCATCATCAAGGACAACTCCAAGAGCCAAGTTTTCTTAAA
AATGAACAGTCTGCAAACTGATGACACAGCCATTTACTACTGTGCCAAAC
ATTATTACTACGGTGGTAGCTATGCTATGGACTACTGGGGTCAAGGAACC
TCAGTCACCGTCTCCTCAGGATCCGAACTTCCTACTCAGGGGACTTTCTC
AAACGTTAGCACAAACGTAAGTCCCGCCCCAAGACCCCCCACACCTGCGC
CGACCATTGCTTCTCAACCCCTGAGTTTGAGACCCGAGGCCTGCCGGCCA
GCTGCCGGCGGGGCCGTGCATACAAGAGGACTCGATTTCGCTTGCGACAT
CTATATCTGGGCACCTCTCGCTGGCACCTGTGGAGTCCTTCTGCTCAGCC
TGGTTATTACTCTGTACTGTAATCACCGGAATCGCCGCCGCGTTTGTAAG
TGTCCCAGGGTCGACAAACGGGGCAGAAAGAAACTCCTGTATATATTCAA
ACAACCATTTATGAGACCAGTACAAACTACTCAAGAGGAAGATGGCTGTA
GCTGCCGATTTCCAGAAGAAGAAGAAGGAGGATGTGAACTGAGAGTGAAG
TTCAGCAGGAGCGCAGACGCCCCCGCGTACCAGCAGGGCCAGAACCAGCT
CTATAACGAGCTCAATCTAGGACGAAGAGAGGAGTACGATGTTTTGGACA
AGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGC
CTACAGTGAGATTGGGATGAAAGGCGAGCGCCGGAGGGGCAAGGGGCACG
ATGGCCTTTACCAGGGTCTCAGTACAGCCACCAAGGACACCTACGACGCC
CTTCACATGCAGGCCCTGCCCCCTCGCTAA
PROTEIN of CAR.CD19SL/LH
(SEQ ID NO: 72):
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGSGGGGEVKLQESGPGLVAP
SQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGT
SVTVSSGSELPTQGTFSNVSTNVSPAPRPPTPAPTIASQPLSLRPEACRP
AAGGAVHTRGLDFACDIYIWAPLAGTCGVLLLSLVITLYCNHRNRRRVCK
CPRVDKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR*

NALM-6 genetically modified with a lentiviral vector carrying CAR.CD19 already published (5) have been included in the experiments as NALM6 CAR UPenn (CAR.CD19 LUSH in the lentivirus platform provided by Dr Ruella (5)).

NK cells from HDs, as well as T cells from HDs or Bcp-ALL patients have been genetically modified by retroviral construct carrying anti-human CD19-scFv from FMC63 clone in which V_{H} and V_{L} fragments were joined by a linker represented by one GGGSGGGG (SEQ ID NO: 38) (G3SG4, short linker), in frame with CD8 stalk domain, 16aa sequence derived from human CD34 antigen (ΔCD34; long hinge), CD8 transmembrane domain, 4.1bb and CD3ζ cytoplasmic domain (CAR.CD19 long hinge, CAR.CD19_{LH}). The retroviral vector is a bicistronic construct in which the above-described CAR construct is in frame with the gene cassette coding for the suicide gene inducible caspase 9 (iC9). iC9-CAR.CD19SL/LH retroviral construct has been used also to genetically modify B leukemic cell lines, including DAUDI, RAJI and NALM-6. CAR+ B cell lines were FACS sorted for CAR expression after transduction. NALM-6 cells were also genetically modified with a lentiviral construct carrying anti-human CD19-scFv from FMC63 clone in which V_{H} and V_{L} fragments were joined by a linker represented by (S3G4)3 Flex linker (SGGGGSGGGGSGGGG SEQ ID NO:39), long linker), in frame with CD8 stalk domain (short hinge), CD8 transmembrane domain, 4.1bb and CD3ζ cytoplasmic domain (NALM-6 CAR.CD19 short hinge, CAR.CD19 UPenn; kindly provided by Dr Ruella).

### Activation of the Suicide Gene

To induce the in vitro activation of iC9, cells were treated once with 20 nM of AP1903 (Medchemexpress, Cat. HY-16046). The percentage of CAR+ cells after the AP1903 treatment was evaluated by FACS at the indicated time points. For in vivo experiments, to prove the ability of the suicide gene iC9 to be active in the control of CAR+ leukemia expansion upon activation by AP1903, NSG mice were infused with 0.25×106 iC9.CAR.CD19_{LH}-DAUDI cells genetically modified with a retroviral construct for FF-Luciferase; after tumor engraftment monitored by IVIS imaging system, the dimerizing drug AP1903 was intraperitoneally administrated from day 1 to day 28 (100μg/mouse). Control cohort was infused with sterile PBS as vehicle solution. Tumor was monitored by weekly IVIS imaging analysis.

### Phenotypic Analysis

Flow cytometry analysis was performed to determine cell surface antigens expression; monoclonal antibodies for CD45, CD3, CD19, CD22, CD10, CD34 (all from Becton Dickinson, USA) were combined with different fluorescence according to needs. iC9.CAR.CD19 expression was detected using a mAb directed to hCD34 epitope (anti-CD34 QBend-10 PE from R&D System, USA), or CD19 CAR Detection Reagent (Biotin; Miltenyi, Germany). Flow-cytometry analysis was performed using a BD LSRFortessa X-20 cytometer (BD Biosciences, USA) and analyzed by FACSDiva software (BD Biosciences, USA). FACS-sorting on CAR-transduced tumor cell lines was performed on a FACSAria (BD Biosciences, USA).

DPs were also characterized by either an 11-color or 16-color combination of antibodies plus CD19-FITC human protein, using the EuroFlow standard operating procedures (SOP) for staining of surface markers only, available at www.EuroFlow.org¹⁸. Antibody combination used were both based on a backbone consisting of the EuroFlow BCP-ALL MRD tubes previously described for high-sensitive MRD measurements in B-cell acute lymphoblastic leukemia by flow cytometry¹⁹⁻²¹. to which the anti-CD3 and both anti-CD22 and anti-HLADR antibody reagents were added for staining of transfected and non-transfected T-cells and specific gating of CD19-negative B cell precursors and blasts, respectively. Finally, the anti-CD34 Qbend10 clone (R&D Systems, Minneapolis, MN) and the CD19-FITC human protein (Cytognos SL, Salamanca, Spain) were also added to the reagent staining mix for identification of transfected CAR.CD19 cells.

All specific reagents are listed in Table 1 A, B and C reported below.

TABLE 1A

| Patient | IG/TR | ASO primer |
|---|---|---|
| ALL#1 | IGH | GTCCGCAATTTTTCATTGGTAGTA (SEQ ID NO: 75) |
| | VH4JH5 | |
| | IGK | GCAAGCTACACAATTAAAGGAGAAGATAGT (SEQ ID NO: |
| | VK2Kde | 76) |
| ALL#2 | IGH | TAGAGATCCGGCCTTTTAACTGGAACT (SEQ ID NO: 77) |
| | VH3JH6 | |
| | IGH | GCAGCACCCCCTCAAGCA (SEQ ID NO: 78) |
| | VH3JH5 | |
| ALL#3 | IGH | TGTGCGAAAGATCTTTTTTTATGGTGTATGCTATTTCTT |
| | VH3JH4 | (SEQ ID NO: 79) |
| | TRD | CGTATCCCCCCCCCACA (SEQ ID NO: 80) |
| | VD2DD3 | |
| ALL#4 | TRB | GCCCCGGACTAGCTAGTTTACGA (SEQ ID NO: 81) |
| | VB20JB2.7 | 45 |
| | IGH | ACTGTCCCCGAGGTTGTACTAATG (SEQ ID NO: 82) |
| | VH3JH5 | |
| ALL#5 | IGH | TGCTATACCGGGGGGTG (SEQ ID NO: 83) |
| | VH3JH6 | |
| | TRD | CCCAGTAAGGTCGGTGGAGTC (SEQ ID NO: 84) |
| | VD2DD3 | |
| ALL#6 | TRA | CGTATCCCCCAGGAGAAGCA (SEQ ID NO: 85) |
| | VD2JA29 | |
| | IGH | ATAGATGTGTACTACTGTGCGAGCGTACTA (SEQ ID NO: |
| | VH1JH4 | 86) |
| ALL#7 | IGH | TCCGGTTGGTATCACCTATCCCCTAA (SEQ ID NO: 87) |
| | VH4JH4 | |
| | TRD | TGTGCGTATCCCCCAGAGACA (SEQ ID NO: 88) |
| | VD2DD3 | |
| ALL#8 | IGH | TGGGTATAACTGGAACTACGGCTGGTT (SEQ ID NO: 89) |
| | DH1JH5 | |
| ALL#9 | IGH | CGGATTTAACTGGGGATCTCCCCTTA (SEQ ID NO: 90) |
| | VH1JH4 | |
| | TRD | CCCCTCCACTCCCCCG (SEQ ID NO: 91) |
| | VD2DD3 | |
| ALL#10 | IGK | CAAGGTACACACTGGCTGGGAA (SEQ ID NO: 92) |
| | VK2Kde | |
| | IGH | CTGCCGACCCACTACATGGA (SEQ ID NO: 93) |
| | VH3JH6 | |
| ALL#11 | IGH | TATAACAGCTCTACTTCTACCACACGACCTA (SEQ ID NO: |
| | VH3JH6 | 94) |
| | TRD | CTACGTGGAACCGTGAGGCT (SEQ ID NO: 95) |
| | DD2DD3 | |
| ALL#12 | TRD | CGTATCCCCCAGTCGCACA (SEQ ID NO: 96) |
| | VD2DD3 | |
| | IGH | CGGAGGGTAAATTACTATGATAGTAGTGGTTT (SEQ ID |
| | VH3JH4 | NO: 97) |
| ALL#13 | IGH | AAAAGGGTCTTGGGCGTTTAGGA (SEQ ID NO: 98) |
| | VH3JH4 | |
| | TRD | GTCCGTACCCCTTGCCG (SEQ ID NO: 99) |
| | VD2DD3 | |
| ALL#14 | IGH | AGTTCCTATCCGAGACCTCCAATT (SEQ ID NO: 100) |
| | VH2JH4 | |
| | TRA | AATTCGGGAGTCGGGGGTAT (SEQ ID NO: 101) |
| | VD2JA29 | |
| ALL#15 | IGH | AGAGAGGAGAGCCTAGGGATATTTTGA (SEQ ID NO: |
| | VH4JH6 | 102) |
| ALL#16 | IGH | GCGAGCAACAACTGGATTTTGA (SEQ ID NO: 103) |
| | VH1JH4 | |
| | TRG | GAACCAACCTCCGAGGCCT (SEQ ID NO: 104) |
| | VG9JG1.3 | |
| ALL#17 | IGH | GTTAATATGGGGCCATCTGGG (SEQ ID NO: 105) |
| | VH2JH3 | |
| ALL#18 | IGH | AGAGGGGGCTCCCCTATGG (SEQ ID NO: 106) |
| | VH1JH3 | |
| | IGH | AGCAGTGGCATGCCATTGA (SEQ ID NO: 107) |
| | VH3JH4 | |
| ALL#19 | TRD | CCTGCCCCCCGCTACAA (SEQ ID NO: 108) |
| | VD2DD3 | |

TABLE 1B

| | | RQ | |
|---|---|---|---|
| Patient | IG/TR | primer | |
| ALL#1 | IGH | jh5 rp2 | CAAGCTGAGTCTCCCTAAGTGGA |
| | VH4JH5 | | (SEQ ID NO: 109) |
| | IGK | kde rp2 | ATATGGCAAAAATGCAGCTGC |
| | VK2Kde | | (SEQ ID NO: 110) |
| ALL#2 | IGH | jh6 rp | GCAGAAAACAAAGGCCCTAGAGT |
| | VH3JH6 | | (SEQ ID NO: 111) |
| | IGH | jh5 rp2 | CAAGCTGAGTCTCCCTAAGTGGA |
| | VH3JH5 | | (SEQ ID NO: 112) |
| ALL#3 | IGH | jh4 rp | CAGAGTTAAAGCAGGAGAGAGGTT |
| | VH3JH4 | | GT (SEQ ID NO: 113) |
| | TRD | vd2 fp | TGCAAAGAACCTGGCTGTACTTAA |
| | VD2DD3 | | (SEQ ID NO: 114) |
| ALL#4 | TRBVB20 | jb2.7 | GCTGGAAGGTGGGGAGA |
| | JB2.7 | rp | (SEQ ID NO: 115) |
| | IGH | jh5 rp2 | CAAGCTGAGTCTCCCTAAGTGGA |
| | VH3JH5 | | (SEQ ID NO: 116) |
| ALL#5 | IGH | jh6 rp | GCAGAAAACAAAGGCCCTAGAGT |
| | VH3JH6 | | (SEQ ID NO: 117) |
| | TRD | vd2 fp | TGCAAAGAACCTGGCTGTACTTAA |
| | VD2DD3 | | (SEQ ID NO: 118) |
| ALL#6 | TRA | vd2 fp | TGCAAAGAACCTGGCTGTACTTAA |
| | VD2JA29 | | (SEQ ID NO: 119) |
| | IGH | jh4 rp | CAGAGTTAAAGCAGGAGAGAGGTT |
| | VH1JH4 | | GT (SEQ ID NO: 120) |
| ALL#7 | IGH | jh4 rp | CAGAGTTAAAGCAGGAGAGAGGTT |
| | VH4JH4 | | GT (SEQ ID NO: 121) |
| | TRD | vd2 fp | TGCAAAGAACCTGGCTGTACTTAA |
| | VD2DD3 | | (SEQ ID NO: 122) |
| ALL#8 | IGH | jh5 rp2 | CAAGCTGAGTCTCCCTAAGTGGA |
| | DH1JH5 | | (SEQ ID NO: 123) |
| ALL#9 | IGH | jh4 rp | CAGAGTTAAAGCAGGAGAGAGGTT |
| | VH1JH4 | | GT (SEQ ID NO: 124) |
| | TRD | vd2 fp | TGCAAAGAACCTGGCTGTACTTAA |
| | VD2DD3 | | (SEQ ID NO: 125) |
| ALL#10 | IGK | kde rp2 | ATATGGCAAAAATGCAGCTGC |
| | VK2Kde | | (SEQ ID NO: 126) |
| | IGH | jh6 rp | GCAGAAAACAAAGGCCCTAGAGT |
| | VH3JH6 | | (SEQ ID NO: 127) |
| ALL#11 | IGH | jh6 rp | GCAGAAAACAAAGGCCCTAGAGT |
| | VH3JH6 | | (SEQ ID NO: 128) |
| | TRD | dd3 rp1 | TTTGCCCCTGCAGTTTTTGT |
| | DD2DD3 | | (SEQ ID NO: 129) |
| ALL#12 | TRD | vd2 fp | TGCAAAGAACCTGGCTGTACTTAA |
| | VD2DD3 | | (SEQ ID NO: 130) |
| | IGH | jh4 rp | CAGAGTTAAAGCAGGAGAGAGGTT |
| | VH3JH4 | | GT (SEQ ID NO: 131) |
| ALL#13 | IGH | jh4 rp | CAGAGTTAAAGCAGGAGAGAGGTT |
| | VH3JH4 | | GT (SEQ ID NO: 132) |
| | TRD | vd2 fp | TGCAAAGAACCTGGCTGTACTTAA |
| | VD2DD3 | | (SEQ ID NO: 133) |
| ALL#14 | IGH | jh4 rp | CAGAGTTAAAGCAGGAGAGAGGTT |
| | VH2JH4 | | GT (SEQ ID NO: 134) |
| | TRA | vd2 fp | TGCAAAGAACCTGGCTGTACTTAA |
| | VD2JA29 | | (SEQ ID NO: 135) |
| ALL#15 | IGH | jh6 rp | GCAGAAAACAAAGGCCCTAGAGT |
| | VH4JH6 | | (SEQ ID NO: 136) |
| ALL#16 | IGH | jh4 rp | CAGAGTTAAAGCAGGAGAGAGGTT |
| | VH1JH4 | | GT (SEQ ID NO: 137) |
| | TRG | vg9 fp | GGCATTCCGTCAGGCAAA |
| | VG9JG1.3 | | (SEQ ID NO: 138) |
| ALL#17 | IGH | jh3 rp | AGGCAGAAGGAAAGCCATCTTAC |
| | VH2JH3 | | (SEQ ID NO: 139) |
| ALL#18 | IGH | jh3 rp | AGGCAGAAGGAAAGCCATCTTAC |
| | VH1JH3 | | (SEQ ID NO: 140) |
| | IGH | jh4 rp | CAGAGTTAAAGCAGGAGAGAGGTT |
| | VH3JH4 | | GT (SEQ ID NO: 141) |
| ALL#19 | TRD | dd3 rp3 | CTGCTTGCTGTGTTTGTCTCCT |
| | VD2DD3 | | (SEQ ID NO: 142) |

TABLE 1C

| | | TaqMan | |
|---|---|---|---|
| Patient | IG/TR | Probe | |
| ALL#1 | IGH | jh1.2.4.5 tp | CCCTGGTCACCGTCTCCTCAGGTG(SEQ ID NO: 143) |
| | VH4JH5 | | |
| | IGK | kde tp1 | AGCCCAGGGCGACTCCTCATGAGT(SEQ ID NO: 144) |
| | VK2Kde | | |
| ALL#2 | IGH | jh6 tp | CACGGTCACCGTCTCCTCAGGTAAGAA(SEQ ID |
| | VH3JH6 | | NO: 145) |
| | IGH | jh1.2.4.5 tp | CCCTGGTCACCGTCTCCTCAGGTG(SEQ ID NO: 146) |
| | VH3JH5 | | |
| ALL#3 | IGH | jh1.2.4.5 tp | CCCTGGTCACCGTCTCCTCAGGTG(SEQ ID NO: 147) |
| | VH3JH4 | | |
| | TRD | vd2 tp | AGACCCTTCATCTCTCTCTGATGGTGCAAGTA(SEQ ID |
| | VD2DD3 | | NO: 148) |
| ALL#4 | TRBVB20 | jb2.7 tp | CGGGCACCAGGCTCACGGTC(SEQ ID NO: 149) |
| | JB2.7 | | |
| | IGH | jh1.2.4.5 tp | CCCTGGTCACCGTCTCCTCAGGTG(SEQ ID NO: 150) |
| | VH3JH5 | | |
| ALL#5 | IGH | jh6 tp | CACGGTCACCGTCTCCTCAGGTAAGAA(SEQ ID |
| | VH3JH6 | | NO: 151) |
| | TRD | vd2 tp | AGACCCTTCATCTCTCTCTGATGGTGCAAGTA(SEQ ID |
| | VD2DD3 | | NO: 152) |
| ALL#6 | TRA | vd2 tp | AGACCCTTCATCTCTCTCTGATGGTGCAAGTA(SEQ ID |
| | VD2JA29 | | NO: 153) |
| | IGH | jh1.2.4.5 tp | CCCTGGTCACCGTCTCCTCAGGTG(SEQ ID NO: 154) |
| | VH1JH4 | | |
| ALL#7 | IGH | jh1.2.4.5 tp | CCCTGGTCACCGTCTCCTCAGGTG(SEQ ID NO: 155) |
| | VH4JH4 | | |
| | TRD | vd2 tp | AGACCCTTCATCTCTCTCTGATGGTGCAAGTA(SEQ ID |
| | VD2DD3 | | NO: 156) |
| ALL#8 | IGH | jh1.2.4.5 tp | CCCTGGTCACCGTCTCCTCAGGTG(SEQ ID NO: 157) |
| | DH1JH5 | | |
| ALL#9 | IGH | jh1.2.4.5 tp | CCCTGGTCACCGTCTCCTCAGGTG(SEQ ID NO: 158) |
| | VH1JH4 | | |
| | TRD | vd2 tp | AGACCCTTCATCTCTCTCTGATGGTGCAAGTA(SEQ ID |
| | VD2DD3 | | NO: 159) |
| ALL#10 | IGK | kde tp1 | AGCCCAGGGCGACTCCTCATGAGT(SEQ ID NO: 160) |
| | VK2Kde | | |
| | IGH | jh6 tp | CACGGTCACCGTCTCCTCAGGTAAGAA(SEQ ID |
| | VH3JH6 | | NO: 161) |
| ALL#11 | IGH | jh6 tp | CACGGTCACCGTCTCCTCAGGTAAGAA(SEQ ID |
| | VH3JH6 | | NO: 162) |
| | TRD | dd3 tp1 | CGCACAGTGCTACAAAACCTACAGAGACCTG(SEQ ID |
| | DD2DD3 | | NO: 163) |
| ALL#12 | TRD | vd2 tp | AGACCCTTCATCTCTCTCTGATGGTGCAAGTA(SEQ ID |
| | VD2DD3 | | NO: 164) |
| | IGH | jh1.2.4.5 tp | CCCTGGTCACCGTCTCCTCAGGTG(SEQ ID NO: 165) |
| | VH3JH4 | | |
| ALL#13 | IGH | jh1.2.4.5 tp | CCCTGGTCACCGTCTCCTCAGGTG(SEQ ID NO: 166) |
| | VH3JH4 | | |
| | TRD | vd2 tp | AGACCCTTCATCTCTCTCTGATGGTGCAAGTA(SEQ ID |
| | VD2DD3 | | NO: 167) |
| ALL#14 | IGH | jh1.2.4.5 tp | CCCTGGTCACCGTCTCCTCAGGTG(SEQ ID NO: 168) |
| | VH2JH4 | | |
| | TRA | vd2 tp | AGACCCTTCATCTCTCTCTGATGGTGCAAGTA(SEQ ID |
| | VD2JA29 | | NO: 169) |
| ALL#15 | IGH | jh6 tp | CACGGTCACCGTCTCCTCAGGTAAGAA(SEQ ID |
| | VH4JH6 | | NO: 170) |
| ALL#16 | IGH | jh1.2.4.5 tp | CCCTGGTCACCGTCTCCTCAGGTG(SEQ ID NO: 171) |
| | VH1JH4 | | |
| | TRG | vg9 tp | TAGGATACCTGAAACGTCTACATCCACTCTCACC(SEQ |
| | VG9JG1.3 | | ID NO: 172) |
| ALL#17 | IGH | jh3 tp | CAAGGGACAATGGTCACCGTCTCTTCA(SEQ ID |
| | VH2JH3 | | NO: 173) |
| ALL#18 | IGH | jh3 tp | CAAGGGACAATGGTCACCGTCTCTTCA(SEQ ID |
| | VH1JH3 | | NO: 174) |
| | IGH | jh1.2.4.5 tp | CCCTGGTCACCGTCTCCTCAGGTG(SEQ ID NO: 175) |
| | VH3JH4 | | |
| ALL#19 | TRD | dd3 tp2 | ATATCCTCACCCTGGGTCCCATGCC(SEQ ID NO: |
| | VD2DD3 | | 176) |

Sample acquisition was performed immediately after sample preparation was completed, >1.5×10⁶ cells (range: 1.6−7.5×10⁶ cells) were measured per sample using an LSRFortessa X-20 [Becton Dickinson Biosciences (BD), San Jose, CA] flow cytometer and the FACSDiva software (BD) or a 3-laser Aurora (Cytek Biosciences, Fremont, CA) spectral flow cytometer equipped with the SpectroFlo software (Cytek). For instrument setup and data acquisition, the EuroFlow SOP for instrument setup and calibration available at www.euroflow.org was strictly followed DOI: 10.1038/leu.2012.122 The Infinicyt software (Cytognos SL, Salamanca, Spain) was used for data analysis.

### Quantitative Real-Time PCR

Total DNA was purified by QIAamp DNA Mini Kit (Qiagen, USA) according to manufacturer.

### Quantitative Real-Time PCR

The average of vector copy number (VCN) per cell was determined by real-time PCR, using a TaqMan probe designed on the retroviral construct using the Primer Express® software (Applied Biosystems) and reported in Table 2 (iC9 Probe and primers iC9).

TABLE 2

| Gene | iC9 |
|---|---|
| Forward | 5′-ACCAGCTGGATGCCATCTC-3′ |
| primer | (SEQ ID NO: 177) |
| Reverse | 5′-CAGCTGCCTGACTTTGGATC-3′ |
| primer | (SEQ ID NO: 178) |
| TaqMan | 5′AGCCTGCCC/ZEN/ACACCTTCTG |
| Probe | ACAT 3′ (SEQ ID NO: 179) |
| | having 5HEX in 5′ and |
| | 3IABKFQ in 3′ |

TaqMan primer/probes were designed for each specific Immunoglobulin (IG) or T-cell Receptor (TR) clonal target by Primer Express® software (Applied Biosystems, Italy).

For VCN, each sample was analyzed in triplicate and the average of threshold cycles was used to quantify DNA copies in relation to the mean values of negative control samples. Relative gene expression was calculated using the house-keeping gene ACT1N1 (Hs_02249516 ACT1N1, ThermoFisher Scientific) qPCR was performed employing QuantStudio 12K Flex Real-Time PCR System (ThermoFisher Scientific). For IG/TR PCR-MRD of the DPs, each MRD value was calculated from the corresponding standard curve, and the results were normalized for values of the housekeeping Albumin gene. Quantitative range (QR) and sensitive range (SR), positive value, reproducibility of replicates, were interpreted following the Euro MRD guidelines, in order to assign the appropriate MRD value to each sample analyzed. qPCR was performed by using the 7900 HT fast-Real Time-PCR System and ViiA7 system (ThermoFisher Scientific) and TaqMan Gene Expression Master Mix (ThermoFisher Scientific).

### In vivo CAR+ Leukemia Mouse Model

Cg-Prkdc^{scid} II2rg^{tm1Wjl}/SzJ (NSG) female mice were provided by Charles River and maintained in the Plaisant animal facility in Castel Romano, Rome Italy. All procedures were performed in accordance with the Guidelines for Animal Care and Use of the National Institutes of Health (Ethical committee for animal experimentation Prot. N 088/2016-PR). To test CAR T activity on CAR+leukemia, the NSG mouse model was intravenously engrafted with 0.25×10⁶ NALM-6 WT or NALM-6 CAR.CD19SULH or NALM-6 CAR.CD19_{LL/SH} or DAUDI CAR.CD19SULH cells genetically modified with firefly luciferase (FF-Luc). On day +3 mice were treated with 10×10⁶ CAR.CD19 T-cells or control untrasduced (NT) T-cells. Tumor growth was monitored weekly by IVIS Imaging System, after D-Luciferin (PerkinElmer, D-Luciferin potassium salt) intraperitoneally administration.

### Statistical Analysis

Unless otherwise noted, data are summarized as average ±standard deviation (SD). Student t-test (two-sided) was used to determine statistically significant differences between samples, with p value <0.05 indicating a significant difference. The mouse survival data were analyzed using the Kaplan-Meier survival curve and Fisher's exact test was used to measure statistically significant differences. No valuable samples were excluded from the analyses. Animals were excluded only in the event of their death after tumor implant but before treatment. Neither randomization nor blinding was done during the in vivo study. However, mice were matched based on the tumor signal for control and treatment groups before infusion of control or specific. To compare the growth of tumors over time, bioluminescence signal intensity was collected in a blind fashion. Bioluminescence signal intensity was log transformed and then compared using a two-sample t-test. The sample size was estimated considering no significant variation within each group of data. A conclusion using as small a sample size as possible was tried to be reached. The sample size to detect a difference in averages of 2 standard deviation at the 0.05 level of significance with an 80% power was estimated. Graphic representations and statistical analysis were performed using GraphPad Prism 6 (GraphPad Software, La Jolla, CA).

### Results

### Generation of CAR.CD19 T-cells from PB Mononuclear Cells of Patients Affected by Bcp-ALL

The un-fractioned population of PB mononuclear cells was isolated from patients at diagnosis having median 45.05±28.12% circulating blasts (n=10. Range, 5.5-86.4%). Mononuclear cells derived from PB of the enrolled patients were transduced with a second-generation iC9.CAR.CD19 long hinge (iC9.CAR.CD19LH) according to the method detailed in FIG. 1A. After 5 days from transduction process, T-cell products showed a transduction efficiency of 55,15±16,54% (FIG. 1B shows an exemplificative analysis, whereas FIG. 1C shows the average of 15 leukemic patients subdivided in two groups based on the % CD19+ cells in the starting material, considering the 45% median value cut-off). It was evaluated whether the leukemic blast contamination in the patient's sample has any impact on the production of CAR T-cells, especially in terms of CAR transduction level in the DPs as well as the total number of generated CAR T-cells. No correlation was noticed between CAR T-cell percentage at the end of the manufacturing procedure and the level of CD19+ B cell contaminating the starting material (FIG. 1C and 1D), as well as on the CAR T-cell yield observed when the manufacturing started from patient's material characterized by more than 45% CD19+ cells (cut-off media value; FIG. 1E). To consider patient's samples with an increased percentage of leukemic blast cells, as well as leukemic cells with a more immature stage, CAR T-cells were generated starting from BM aspirate samples of Bcp-ALL patients at diagnosis (n=6. FIG. 2A) in which the average of CD19+ cells in the starting material was 73.1±17,80% (range, 40.5-83.5%). Although the transduction level in BM-derived samples was significantly lower than in PB-samples (FIG. 2A-B; 36,04±19,83% vs 55,15±16,54% CAR+ T cells, respectively; p=0.03), no differences were observed in terms of total yield of DP recovery from PB or BM samples (FIG. 2C).

### Deep Characterization of Patient-derived CAR T-cell Products

Real-time quantitative PCR was performed on CAR T-cell DPs (day 14, end-production) for the amplification of patient-specific lg rearrangements, observed at diagnosis of each patient. Positivity of MRD in 7 out of 14 tested CAR T cell DPs was observed, with a median MRD of 6.01E-3±1.00E-2 (Table 3).

TABLE 3

| ALL Patients | % CD19+ | | | |
|---|---|---|---|---|
| (PB) | Blasts | Sample | MRD#1 | MRD#2 |
| ALL#7 | 86.4 | NT | 1.00E−04 | NEG |
| | | CAR | 1.20E−04 | NEG |
| ALL#8 | 28.5 | NT | NEG | ND |
| | | CAR | NEG | ND |
| ALL#9 | 5.5 | NT | NEG | NEG |
| | | CAR | NEG | NEG |
| ALL#4 | 28.7 | NT | ND | ND |
| | | CAR | NEG | NEG |
| ALL#10 | 44.8 | NT | 9.40E−03 | 2.30E−03 |
| | | CAR | 8.40E−03 | 1.90E−03 |
| ALL#6 | 75.3 | NT | NEG | NEG |
| | | CAR | NEG | NEG |
| ALL#11 | 13.9 | NT | 1.10E−03 | 6.00E−04 |
| | | CAR | NEG | NEG |
| ALL#12 | 79.8 | NT | 1.50E−04 | 1.00E−04 |
| | | CAR | 3.50E−04 | 1.30E−04 |
| ALL#13 | 33.6 | NT | NEG | NEG |
| | | CAR | NEG | ND |
| ALL#14 | 36.8 | NT | 1.2E−04 | NEG |
| | | CAR | NEG | NEG |
| ALL#15 | 48.3 | NT | 1.9E−04 | ND |
| | | CAR | 6.4E−04 | ND |
| ALL#16 | 75.2 | NT | 3.4E−03 | 3.5E−03 |
| | | CAR | 2.7E−03 | 2.1E−02 |
| ALL#18 | 36.4 | NT | 3.4E−04 | 6.2E−04 |
| | | CAR | 3.0E−04 | NEG |
| ALL#19 | 94.1 | NT | 3.8E−03 | ND |
| | | CAR | 2.1E−03 | ND |

Table 3 shows data from each enrolled patient as regarding the percentage of CD19+ leukemia cells in the row starting material considered for the CAR T manufacturing, and the value of MRD for two different Ig markers (MRD #1 and MRD #2) identified at the time of diagnosis in each single patient. MRD data were reported for both control un-transduced T cell samples (NT) and CAR.CD19 T cell samples (CAR).

Moreover, it was observed that the leukemic blast cells contamination was also present in 9 out of 13 tested un-transduced T cell samples (NT, Table 3), proving that leukemic cells survive in culture independently from transduction process. More in depth, a time-course experiments have been performed, in which DPs were analyzed for MRD at very early time point of Day+8 after activation, Day+14 as standard procedures for CAR T manufacturing, and Day+30 as much extended culture for a CAR T-cell DP (data obtained from 5 different patient's productions, n=5). As shown in FIG. 3A, an inverse correlation was observed between MRD levels and the time-course of in vitro culture, with a significant reduction of MRD as the time of culture progressively increase (p=0.02 considering MRD at Day+8 vs Day+14), reaching a level behind the sensitivity at the last time-point of Day+30. For those samples with enough available materials, DP analysis have also been performed by applying the high sensitivity EuroFlow cytometry platform (Leukemia. 2012 September; 26 (9): 1899-1907). As clearly shown in FIG. 3B reporting data from one exemplificative CAR T-cell DP at Day+14 of manufacturing process and control culture of NT T-cells, the applied cytofluorimetric analysis was sensitive enough to detect positive MRD. B cell precursors contaminating

CAR products resulted to be CD19 very dull (FIG. 4) but preserved other B cell markers, as CD10 (FIG. 4). Nevertheless, the percentage of B cells contaminating in vitro expanded NT T-cells, resulting CD19+CD10+, was significantly higher than those observed in CAR samples (FIG. 4, MRD 1.5% vs 0.00036%, respectively). It was verified whether B cells were characterized by CAR transduction. As shown in FIG. 5, for two samples with positive MRD in PCR and then analyzed by cytofluorimetric assay, CAR positive leukemia cells were detected, showing a double positivity to two different staining for CAR molecule (anti-CD34 detecting epitope on the hinge region of iC9.CAR.CD19_{LH} as well as CD19 epitope recognized by CAR.CD19 scFv). Data were also confirmed on BM-derived DPs, in which RT-qPCR show positivity of MRD in 6 out of 6 CAR T productions (Table 4).

TABLE 4

| ALL Patients | % CD19+ | | | |
|---|---|---|---|---|
| (BM) | Blasts | Sample | MRD#1 | MRD#2 |
| ALL#1 | 80.7 | NT | 6.03E−03 | 7.50E−03 |
| | | CAR | 3.60E−03 | 5.20E−03 |
| ALL#2 | 40.5 | NT | 7.60E−03 | 5.20E−03 |
| | | CAR | 9.90E−03 | 7.00E−03 |
| ALL#3 | 50.9 | NT | 2.60E−03 | 4.20E−03 |
| | | CAR | 5.40E−02 | 5.40E−02 |
| ALL#4 | 75.6 | NT | 2.80E−03 | 2.60E−03 |
| | | CAR | 2.30E−03 | 1.90E−03 |
| ALL#5 | 83.9 | NT | 1.20E−03 | ND |
| | | CAR | 1.20E−03 | 1.90E−04 |
| ALL#6 | 83.5 | NT | ND | ND |
| | | CAR | POS < QR | NEG |

Table 4 shows data of each enrolled patient as regarding the percentage of CD19+ leukaemia cells in the patient derived BM mononuclear cells used as starting row material for the CAR T cell manufacturing, and the value of MRD for two different Ig markers identified at the time of diagnosis in each single patient. MRD data were reported for both control un-transduced T cell samples (NT) and iC9.CAR.CD19LH T cell samples (CAR).

For two BM-derived DPs the MRD was also confirmed by EuroFlow cytometry platform (FIG. 5). In these cases, the sensitivity of the assay and the quality of frozen samples did not allow the detection of CAR positive B cell precursors. Also in BM-derived CAR T cell products, B-cell precursors contaminating the DPs were CD19 dim/negative, whereas in un-transduced T-cell products contaminating B cells showed a high expression of CD19.

In summary, the proof has been provided that if a deep characterization of the drug product is carried out, the occurrence of leukemia CAR+ B cells is often detectable, underlined an urgent need to improve the safety profile of the CAR construct as high number of patients will soon treated with CAR T cells.

### The Length of the CAR Linker Influences Epitope Masking

Whether CAR.CD19 design in the linker and the hinge regions could have any impact on the CD19 masking when CAR.CD19 is co-expressed with CD19 on the same cellular membrane has been evaluated.

In particular, four specific conformations, summarized in FIG. 6A, were considered to demonstrate which configuration conformation in the CAR construct is responsible for the CD19 antigen masking in CAR+ leukemia cells. To this end, the 4 different CAR constructs were used to genetically modify NALM-6 cell line. Although the cells maintained CD19 positivity at the mRNA level (FIG. 7A), the pattern of CD19-associated fluorescence levels in NALM-6 CAR.CD19LL/SH (light gray histogram) was superimposable to the control isotype (dark gray histogram) (FIG. 6B), confirming previously published data (5). Then, a different second-generation CAR.CD19 configuration has been considered characterized by a long linker (as the one before), but in the presence of a long hinge that includes ACD34 (CAR.CD19LL/LH). In this case, masking flow-cytometry analysis showed no difference compared to the reference CAR.CD19LL/SH (FIG. 6C). Then the contribution of the linker length between VL and VH regions has been evaluated, considering the MFI detection of CD19 in NALM-6 carrying CAR.CD19SL/SH. As shown in FIG. 6D, CD19 MFI was higher in NALM-6 CAR.CD19SL/SH with respect to the reference CAR.CD19LL/SH. Finally, NALM-6 genetically modified with the CAR construct of the present invention has been considered in which the short linker between VL and VH is associated to the long hinge that includes ACD34, as trackable marker for CAR T cells.

Also in the case of a LH, the SH is associated to a different CD19 MFI respect to the reference structure (FIG. 6E). The same data were also observed in other two different B cell lines, DAUDI and RAJI cells, in which an un-complete CD19 masking has been shown when lymphoma cells were genetically modified with CAR.CD19SL/LH (FIG. 7B). Based on these observations, it has been speculated that the linker length could be the factor driving a complete or un-complete CD19 antigen CIS masking on CAR+ leukemic cells in the retroviral platform. These results were also corroborated by functional analysis. Indeed, very low level of CD19 expression on DAUDI, RAJI and NALM-6 CAR.CD19SL/LH cells was sufficient to elicit CAR.CD19 T-cell response, although to a lower extent if compared to wild-type cell lines (FIG. 8A-C), particularly at low effector/target ratios. As shown in FIG. 8C, CAR.CD19 T cells exert a complete leukaemia control against NALM-6 WT, with no significant differences compared to the anti-leukaemia activity observed against NALM-6 CAR.CD19SL/SH and NALM-6 CAR.CD19SL/LH. Of note, whereas CAR.CD19 T cells were completely unable to recognize NALM-6 CAR+applied by Ruella et al in the previous publication (5) (FIG. 7C), some activity of CAR.CD19 T cells against NALM-6 CAR.CD19LL/SH has been observed, although to a lower extent compared to NALM-6 CAR.CD19SL/SH and NALM-6 CAR.CD19SL/LH (FIG. 8C). In line with these findings, it has been also observed that NALM-6 cells genetically modified with both CAR.CD19 with SL or LL were able to induce a significant amount of interferon-gamma (IFN-g) by CAR T cells (FIG. 9A), as well as to induce their proliferation (FIG. 9B).

### Short Linker and Long Hinge in CAR.CD19 construct did not impact on CAR functionality and immunogenicity.

Notably, whereas CAR.CD19SL/LH is leading to the un-complete CD19 antigen CIS masking, when expressed on T cells, it was able to exert a significant leukemia/lymphoma control. In particular, co-culture assay was used to demonstrate the cytotoxic effect of CAR.CD19SL/LH T cells against DAUDI (FIG. 8A), Raji (FIG. 8B) and NALM-6 (FIG. 8C) cell line. As shown in FIG. 8A and 8B, CAR.CD19SL/LH T cells are able to eliminate tumor cells from the culture even when used at low effector/target ratio. For the NALM-6 model, also the anti-leukemia activity of CAR.CD19SL/LH T cells have been compared with that of the more standard CAR.CD19LL/SH T cells, showing no substantial differences in terms of cytotoxicity (FIG. 8C, NALM-6 WT), interferon gamma (IFN-g) production (FIG. 9A), or proliferation index after antigen stimulation (FIG. 9B). This last assay was performed by stimulating CFSE loaded CAR T cells with NALM-6 WT cells, and observing that irrespectively of the CAR construct, both CAR.CD19 T cells were able to reach comparable level of high proliferating cells (light grey histograms) respect to un-stimulated cells (dark grey histograms). Moreover, since the trackable marker CD34 has been included in the CAR configuration, in silico analysis has been also performed to predict its immunogenicity. In particular, the peptide sequences that are confidently foreseen to be presented by MHC molecules in the CAR region that include CD34 domain have been studied. “STNVSPAPR” (SEQ ID NO:181 peptide is predicted as potentially immunogenic for CD34-including construct (Table5, presented by the MHC molecules HLA-A11:01 and HLA-A33:03. The peptide “GSELPTQGTF” (SEQ ID NO: 182) also matches the selection criteria, but in this case, its binding core is “ELPTQGTF” (SEQ ID NO: 183) and is entirely part of the CD34 epitope region; therefore, it is unlikely to be highly immunogenic. For the CAR construct in which CD34 domain was not considered, the peptides “SVTVSSPAPR” (SEQ ID NO:184) and its shorter version “VTVSSPAPR” (SEQ ID NO:185) are both predicted to be immunogenic, presented by the same alleles HLA-A11:01 and HLA-A33:03. In light of these data, we predict that the inclusion of CD34 domain in the construct did not substantially impact on the immunogenic profile of the CAR.

TABLE 5

| Peptide | Binding alleles | CAR construct |
|---|---|---|
| VTVSSPAPR | HLA-A11:01, | Short hinge no CD34 |
| (SEQ ID NO: 185) | HLA-A33:03 | |
| SVTVSSPAPR | HLA-A33:03 | Short hinge no CD34 |
| (SEQ ID NO: 184) | | |
| GSELPTQGTF | HLA-B40:01 | Long hinge including |
| (SEQ ID NO: 182) | | CD34 |
| STNVSPAPR | HLA-A11:01, | Long hinge including |
| (SEQ ID NO: 181) | HLA-A33:03 | CD34 |

### The Activation of the Suicide Gene iC9 Controls Expansion of CAR+ Leukemic Cells

The possibility to promptly eliminating CAR+ leukemic cells was demonstrated, through the exposure of DAUDI, RAJI and NALM-6 iC9.CAR+ cells to 20 nM of AP1903. Indeed, very early activation (6 hours) of the suicide gene iC9 corresponded to a significant reduction in the percentage of CAR+ leukemic cells (FIG. 8D and 8E for DAUDI cells and FIG. 10 for RAJI and NALM6 cells). Prolonged culture of AP1903-treated iC9.CAR.CD19 DAUDI cells was not associated to re-expansion of iC9.CAR+ leukemic cells (FIG. 8E). In particular, the MFI of CAR expression in AP1903 treated cells was equal to 142±22 (threshold value; FIG. 10E), a value significantly inferior as compared to un-treated cells, but higher than the CAR staining of DAUDI WT cells (125.8±20.6, FIG. 10E). The same results were also confirmed in RAJI (FIG. 10A-B) and NALM-6 (FIG. 10C-D) cellular models. While the presence of leukemic cells with high CAR expression MFI was undetectable by flow-cytometry analysis in AP1903 treated cells, the presence of leukemic cells was observed with very dim (i.e. moderate) expression of CAR.CD19, but a completed re-established detection of CD19 antigen, as in wild-type cell lines (FIG. 8E, 10B and 10D). Indeed, qPCR analysis reveals the detection of the transgene (TG) in the remaining cells after AP1903 exposure (FIG. 9F), although significantly decreased respect to untreated CAR+ cells (TG positivity was observed in 22.8% of DAUDI cells, 18.6% of RAJI cells, and 0.6% of NALM-6 cells). Vector Copy Number analysis reveals that AP1903 treated residual cells had a significantly lower number of inserted vector compared to the untreated one (5.3±4.2 and 0.1±0.1 average of VCN threshold in untreated and AP1903 treated B cells, respectively, across all the considered cellular models; FIG. 10F). Since CD19 detection was completely re-established in iC9.CAR+ B cells rescued after AP1903 exposure, whether they could be targeted by CAR.CD19 T-cells has been verified. As shown in FIG. 11, CAR.CD19 T-cells (FIG. 11A and 11B) were able to eliminate CAR+ leukemic cells spared by AP1903 treatment. Moreover, because of the clinical unfeasibility to generate an autologous CAR T-cell product from patients with a CAR+ B cell relapse, it has been also proved that healthy donor-derived CAR.CD19 NK-cells are able to significantly control iC9.CAR+ B cells rescued after AP1903 exposure (FIG. 11C and 11D). A double strategy for the in vivo control of a CAR+ leukemia. The ability of CAR.CD19 T cells to target wild-type B cell leukemia beside CAR construct with a SL or a LL, has been proved in vivo in a B cell leukemia NSG xenograft model. In particular, mice were infused systemically with NALM-6 genetically modified with FF-luciferase to allow in vivo monitoring of the leukaemia burden overtime. Tumour engraftment was analyzed by measuring the bioluminescence signal, and, on Day+0, mice were treated with both CAR.CD19SL/LH and CAR.CD19LL/SH T cells, as well as with control NT-T-cells derived from HDs (FIG. 12A). CAR.CD19SL/LH and CAR.CD19LL/SH T cells were able to significantly control NALM-6 in vivo expansion, as clearly demonstrated by bioluminescence analysis. Then whether NALM-6 CAR.CD19SL/LH were also recognized by CAR T cells in the in vivo setting has been evaluated. As shown in FIG. 12B, CAR.CD19SL/LH T cells were able to reduce significantly CAR+ NALM-6 cell in vivo expansion as compared to control NT T cells. The same data were also confirmed in a less aggressive lymphoma model of DAUDI cell line (FIG. 12D). In this model, CAR.CD19SL/LH T cells were able to control and eliminate CAR+ lymphoma cells in all treated mice. The mouse cohort treated with CAR.CD19 T-cells reached 100% disease-free survival (DFS) at the end of the experimental procedure (day 21) vs 0% DFS for the control cohort of mice receiving NT T-cells. Moreover, the in vitro data relative to NALM-6 CAR.CD19LL/SH have been also corroborated. Also in the in vivo setting, CAR T cells were able to exert anti-leukemia control against NALM-6 CAR.CD19LL/SH (FIG. 12C), although to a lower extent of those observed in CAR.CD19SL/LH model (FIG. 12B and FIG. 12E).

Finally, the ability of the suicide gene iC9 to be active in the control of CAR+ leukemia expansion upon activation by AP1903 was also proved in vivo. In particular, NSG mice were infused with iCas9.CAR.CD19_{LH}

DAUDI cells; after tumor engraftment, the dimerizing drug AP1903 was intraperitoneally administrated from day 1 to day 28 (FIG. 13A). The activation of iC9 by administration of AP1903 resulted in a complete CAR+ leukemia eradication in 9 out of 10 studied mice (FIG. 13B and 13C). Moreover, AP1903 administration allow the survival of 100% of the treated mice even after drug administration suspension, with no mice showing leukemia recurrence until day 63 (endpoint of the experiment; FIG. 13D). The only mouse showing a positive signaling in IVIS analysis after CID administration, was early sacrificed at day 35 without sign of suffering together with a negative control (mouse of the same cohort) and a positive control (mouse without CID administration), to characterize the leukemia cells. By applying citofluorimetric analysis on peripheral blood, spleen, and tibias BM (left flank), leukemia cells could not be detected in mice treated with CID with a positive expression of CAR molecule.

### EXAMPLE 2

### Comparison of Different CAR.CD19 Molecules with Short Linker from 8 aa to 14 aa and Long Linker

In silico model has been performed to demonstrate that CAR.CD19 molecules conceived with a short linker spanning from 8 to 14 aa are characterized by a 3D structure different compared to the classical CAR.CD19 with a long linker of 15aa, providing an additional proof that CAR.CD19s with a linker with a reduced length have a spatial configuration providing a different masking of the target respect to that observed in CAR.CD19 with a standard linker of 15aa.

The following linkers have been used in order to link VL sequence SEQ ID NO:15 and VH sequence SEQ ID NO:16:

(SEQ ID NO: 38)
G3SG4 GGGSGGGG short linker
(SEQ ID NO: 186)
SG4SG3 SGGGGSGGG,
(SEQ ID NO: 190)
(SG4)2 SGGGGSGGGG
(SEQ ID NO: 187)
(SG4)2 S SGGGGSGGGGS
(SEQ ID NO: 188)
(SG4)2 SG SGGGGSGGGGSG
(SEQ ID NO: 191)
(SG4)2 SG2 SGGGGSGGGGSGG
(SEQ ID NO: 189)
(SG4)2 SG3 SGGGGSGGGGSGGG
(SEQ ID NO: 39)
(SG4)3 SGGGGSGGGGSGGGG Long linker

For this model, Superpose tool has been used to calculates protein superposition using a modified quaternion approach. From a superposition of two or more structures, Superpose generates sequence alignments, structure alignments, PDB coordinates, RMSD statistics, Difference Distance Plots, and interactive images of the superimposed structures.

The SuperPose web server supports the submission of either PDB-formatted files or PDB accession numbers. This tool has been used to compare the structures of CAR.CD19 that comprises linkers of different lengths spanning the entire repertoire from 8 aa to 15 aa.

The different distance matrix is generated as a PNG image that may be used to visually identify regions where there are significant differences between any structures comprising a linker from 8 to 14 aa, versus the standard CAR.CD19 structure comprising a long linker of 15 aa. The lighter the region, the more similar the structures are (FIGS. 14-21). Likewise, the darker the region, the more different the structure are. The default display for SuperPose's difference distance plot show 6 graded cutoffs.

Differences between 0 and 1,5 Angstroms (A) are white; Differences between 1,5 and 3,0 A are very light gray; Differences between 3,0 and 5,0 A are light gray; Differences between 5 and 7 A are gray; Differences between 7 and 9 A are dark gray; Differences between 9 and 12 A are very dark gray and those greater than 12 A are black.

FIGS. 14-21 show also summarizing tables for the (Root-mean-square deviation) RMSD data relative to alfa carbons and back bone, as well as the heavy structure. These tables show a significant difference of all the CAR.CD19 configurations with a linker spanning from 8 to 14aa, compared to CAR.CD19 with a longer 15aa linker. These differences are suggesting that CAR.CD19 with a linker comprising from 8 to 14aa have a different masking potential respect to the CAR.CD19 with a longer linker. The root-mean-square deviation of atomic positions, or simply root-mean-square deviation (RMSD), is the measure of the average distance between the atoms (usually the backbone atoms) of superimposed proteins.

### References

1 Frey, N. The what, when and how of CAR T cell therapy for ALL. Haematol Best Pract Res Clin 30, 275-281, doi: 10.1016/j.beha.2017.07.009 (2017).
2 Gokbuget, N. How should we treat a patient with relapsed Ph-negative B-ALL and what novel approaches are being investigated? Best Pract Res Clin Haematol 30, 261-274, doi: 10.1016/j.beha.2017.07.010 (2017).
3 Park, J. H. et al. Long-Term Follow-up of CD19 CAR Therapy in Acute Lymphoblastic Leukemia. N Engl J Med 378, 449-459, doi: 10.1056/NEJMoa1709919 (2018).
4 Maude, S. L. et al. Tisagenlecleucel in Children and Young Adults with B-Cell Lymphoblastic Leukemia. N Engl J Med 378, 439-448, doi: 10.1056/NEJMoa1709866 (2018).
5 Ruella, M. et al. Dual CD19 and CD123 targeting prevents antigen-loss relapses after CD19-directed immunotherapies. J Clin Invest 126, 3814-3826, doi: 10.1172/JCl87366 (2016).
6 Brudno, J. N. & Kochenderfer, J. N. Recent advances in CAR T-cell toxicity: Mechanisms, manifestations and management. Blood Rev 34, 45-55, doi: 10.1016/j.blre.2018.11.002 (2019).
7 Wang, Z., Wu, Z., Liu, Y. & Han, W. New development in CAR-T cell therapy. J Hematol Oncol 10, 53, doi: 10.1186/s13045-017-0423-1 (2017).
8 Philip, B. et al. A highly compact epitope-based marker/suicide gene for easier and safer T-cell therapy. Blood 124, 1277-1287, doi: 10.1182/blood-2014-01-545020 (2014).
9 Bonini, C. et al. HSV-TK gene transfer into donor lymphocytes for control of allogeneic graft-versus-leukemia. Science 276, 1719-1724, doi: 10.1126/science.276.5319.1719 (1997).
10 Di Stasi, A. et al. Inducible apoptosis as a safety switch for adoptive cell therapy. N Engl J Med 365, 1673-1683, doi: 10.1056/NEJMoa1106152 (2011).
11 Zhou, X. et al. Inducible caspase-9 suicide gene controls adverse effects from alloreplete T cells after haploidentical stem cell transplantation. Blood 125, 4103-4113, doi: 10.1182/blood-2015-02-628354 (2015).
12 Ruella, M. et al. Induction of resistance to chimeric antigen receptor T cell therapy by transduction of a single leukemic B cell. Nat Med 24, 1499-1503, doi: 10.1038/s41591-018-0201-9 (2018).
13 Ruella, M. et al. A cellular antidote to specifically deplete anti-CD19 chimeric antigen receptor-positive cells. Blood 135, 505-509, doi: 10.1182/blood.2019001859 (2020).
14 Kemper, K., Rodermond, H., Colak, S., Grandela, C. & Medema, J. P. Targeting colorectal cancer stem cells with inducible caspase-9. Apoptosis 17, 528-537, doi: 10.1007/s10495-011-0692-z (2012).
15 Yagyu, S., Hoyos, V., Del Bufalo, F. & Brenner, M. K. An Inducible Caspase-9 Suicide Gene to Improve the Safety of Therapy Using Human Induced Pluripotent Stem Cells. Mol Ther 23, 1475-1485, doi: 10.1038/mt.2015.100 (2015).
16 Zhou, X. et al. Serial Activation of the Inducible Caspase 9 Safety Switch After Human Stem Cell Transplantation. Mol Ther 24, 823-831, doi: 10.1038/mt.2015.234 (2016).
17 Quintarelli, C. et al. Efficacy of third-party chimeric antigen receptor modified peripheral blood natural killer cells for adoptive cell therapy of B-cell precursor acute lymphoblastic leukemia. Leukemia 34, 1102-1115, doi: 10.1038/s41375-019-0613-7 (2020).
18 Kalina, T. et al. EuroFlow standardization of flow cytometer instrument settings and immunophenotyping protocols. Leukemia 26, 1986-2010, doi: 10.1038/leu.2012.122 (2012).
19 Maude S L, Teachey D T, Porter D L, Grupp S A. CD19-targeted chimeric antigen receptor T-cell therapy for acute lymphoblastic leukemia. Blood. 2015;125 (26): 4017-4023. Blood 128, 1441, doi: 10.1182/blood-2016-07-730333 (2016).
20 Theunissen, P. et al. Standardized flow cytometry for highly sensitive MRD measurements in B-cell acute lymphoblastic leukemia. Blood 129, 347-357, doi: 10.1182/blood-2016-07-726307 (2017).
21 Sedek, L. et al. Differential expression of CD73, CD86 and CD304 in normal vs. leukemic B-cell precursors and their utility as stable minimal residual disease markers in childhood B-cell precursor acute lymphoblastic leukemia. J Immunol Methods 475, 112429, doi: 10.1016/j.jim.2018.03.005 (2019).
22 Magdelaine-Beuzelin, C. et al. Structure-function relationships of the variable domains of monoclonal antibodies approved for cancer treatment. Crit Rev Oncol Hematol 64, 210-225,
23 DiJoseph, J. F. et al. Antibody-targeted chemotherapy of B-cell doi: 10.1016/j.critrevonc.2007.04.011 (2007). lymphoma using calicheamicin conjugated to murine or humanized antibody against CD22. Cancer Immunol Immunother 54, 11-24, doi: 10.1007/s00262-004-0572-2 (2005).
24 Kantarjian, H., Thomas, D., Wayne, A. S. & O′Brien, S. Monoclonal antibody-based therapies: a new dawn in the treatment of acute lymphoblastic leukemia. J Clin Oncol 30, 3876-3883, doi: 10.1200/JCO.2012.41.6768 (2012).

1. A chimeric antigen receptor comprising, from the N-terminus to the C-terminus:
a) a signal peptide,
b) a single chain antibody domain chosen from the group consisting of anti CD19 single chain antibody domain, anti CD20 single chain antibody domain or anti CD22 single chain antibody domain, said single chain antibody domain comprising or consisting of VL and VH sequences linked each other by a linker,
c) a hinge,
d) a trans membrane domain,
e) a co-stimulatory signaling domain, and
f) CD3Zeta chain sequence,
wherein said linker is a short flexible linker with a length from 7 to 14 amino acids, such as from 7 to 12, from 7 to 10 or 8 amino acids.

2. The chimeric antigen receptor according to claim 1, wherein:
anti CD19 single chain antibody domain comprises anti CD19 FMC63 hybridoma VL and VH sequences, wherein anti CD19 FMC63 hybridoma VL sequence comprises CDR1 sequence QDISKY (SEQ ID NO:1), CDR2 sequence HTS and CDR3 sequence GNTLP (SEQ ID NO: 2), whereas anti CD19 FMC63 hybridoma VH sequence comprises CDR1 sequence GVSLPDYG (SEQ ID NO:3), CDR2 sequence IWGSETT (SEQ ID NO:4) and CDR3 sequence AKHYYYGGSYAMDY (SEQ ID NO:5);
anti CD20 single chain antibody domain comprises anti CD20 VL and VH sequences, wherein anti CD20 VL sequence comprises CDR1 sequence SSVSY (SEQ ID NO:6), CDR2 sequence ATS and CDR3 sequence QQWTSNPPT (SEQ ID NO:7), whereas anti CD20 VH sequence comprises CDR1 sequence GYTFTSYN (SEQ ID NO:8), CDR2 sequence IYPGNGDT (SEQ ID NO:9) and CDR3 sequence ARSTYYGGDWYFNV (SEQ ID NO:10);
anti CD22 single chain antibody domain comprises anti CD22 VL and VH sequences, wherein anti CD22 VL sequence comprises CDR1 sequence QSLANSYGNTF (SEQ ID NO:11), CDR2 sequence GIS and CDR3 sequence LQGTHQP (SEQ ID NO:12), whereas anti CD 22 VH sequence comprises CDR1 sequence GYRFTNYWIH (SEQ ID NO:13), CDR2 sequence INPGNNYA (SEQ ID NO:14) and CDR3 sequence TR.

3. The chimeric antigen receptor according to claim 2, wherein:
anti-CD19 FMC63 hybridoma VL sequence comprises or consists of sequence
DIQMTQTTSSLSASLGDRVTISCRASQDISKYLNWYQQKPDGTVKLLIYHTSRLH SGVPSRFSGSGSGTDYSLTISNLEQEDIATYFCQQGNTLPYTFGGGTKLEIT (SEQ ID NO: 15) and
anti-CD19 FMC63 hybridoma VH sequence comprises or consists of sequence
EVKLQESGPGLVAPSQSLSVTCTVSGVSLPDYGVSWIRQPPRKGLEWLGVIWGSE TTYYNSALKSRLTIIKDNSKSQVFLKMNSLQTDDTAIYYCAKHYYYGGSYAMDYWGQG TSVTVSS (SEQ ID NO:16);
anti-CD20 VL sequence comprises or consists of sequence
QIVLSQSPAILSASPGEKVTMTCRASSSVSYIHWFQQKPGSSPKPWIYATSNLASG VPVRFSGSGSGTSYSLTISRVEAEDAATYYCQQWTSNPPTFGGGTKLEIK (SEQ ID NO: 17) and
anti-CD20 VH sequence comprises or consists of sequence
QVQLQQPGAELVKPGASVKMSCKASGYTFTSYNMHWVKQTPGRGLEWIGAIYP GNGDTSYNQKFKGKATLTADKSSSTAYMQLSSLTSEDSAVYYCARSTYYGGDWYFNV WGAGTTVTVSA (SEQ ID NO:18);
anti-CD22 VL sequence comprises or consists of sequence
DVQVTQSPSSLSASVGDRVTITCRSSQSLANSYGNTFLSWYLHKPGKAPQLLIYGI SNRFSGVPDRFSGSGSGTDFTLTISSLQPEDFATYYCLQGTHQPYTFGQGTKVEIK (SEQ ID NO: 19) and
anti-CD22 VH sequence comprises or consists of sequence
EVQLVQSGAEVKKPGASVKVSCKASGYRFTNYWIHWVRQAPGQGLEWIGGINP GNNYATYRRKFQGRVTMTADTSTSTVYMELSSLRSEDTAVYYCTREGYGNYGAWFAY WGQGTLVTVSS (SEQ ID NO:20).

4. The chimeric antigen receptor according to claim 1, wherein the linker which links VL and VH sequences is chosen from the group consisting of a flexible Flex linker glycines-rich, such as (G4S)2 linker GGGGSGGGG (SEQ ID NO:35), G4SG2 linker GGGGSGG (SEQ ID NO:37) or G3SG4 linker GGGSGGGG (SEQ ID NO:38), SG4SG3 linker SGGGGSGGG (SEQ ID NO:186), (SG4)2 S linker SGGGGSGGGGS (SEQ ID NO:187), (SG4)2 SG linker SGGGGSGGGGSG (SEQ ID NO: 188), (SG4)2 SG3 linker SGGGGSGGGGSGGG linker (SEQ ID NO:189), (SG4)2 SGGGGSGGGG (SEQ ID NO:190), (SG4)2 SG2 SGGGGSGGGGSGG (SEQ ID NO:191), preferably, G3SG4 linker.

5. The chimeric antigen receptor according to claim 1, wherein said hinge comprises or consists of one or more of the following hinges:
CD8stalk
TTTPAPRPPTPAPTIASQPLSLRPEACRPAAGGAVHTRGLDFACD (SEQ ID NO:21);
Hinge CD28 EVMYPPPYLDNEKSNGTIIHVKGKHLCPSPLFPGPSKP (SEQ ID NO: 22);
hinge CH2-CH3
ESKYGPPCPSCPAPEFLGGPSVFLFPPKPKDTLMISRTPEVTCVVVDVSQEDPEVQ FNWYVDGVEVHNAKTKPREEQFNSTYRVVSVLTVLHQDWLNGKEYKCKVSNKGLPSS IEKTISKAKGQPREPQVYTLPPSQEEMTKNQVSLTCLVKGFYPSDIA VEWESNGQPENNY KTTPPVLDSDGSFFLYSRLTVDKSRWQEGNVFSCSVMHEALHNHYTQKSLSLSLGK (SEQ ID NO:23); or
hinge CH3:
ESKYGPPCPSCPGQPREPQVYTLPPSQEEMTKNQVSLTCLVKGFYPSDIA VEWES NGQPENNYKTTPPVLDSDGSFFLYSRLTVDKSRWQEGNVFSCSVMHEALHNHYTQKSL SLSLGK (SEQ ID NO:24), preferably CD8stalk.

6. The chimeric antigen receptor according to claim 1, wherein said hinge is linked, at the N terminus, to a trackable marker, said trackable marker being linked, optionally by a second linker, to the single chain antibody domain.

7. Chimerie The chimeric antigen receptor according to claim 6, wherein the trackable marker is chosen from the group consisting of:
ΔCD34 ELPTQGTFSNVSTNVS (SEQ ID NO:25); or
NGFR
KEACPTGLYTHSGECCKACNLGEGVAQPCGANQTVCEPCLDSVTFSDVVSATEP CKPCTECVGLQSMSAPCVEADDAVCRCAYGYYQDETTGRCEACRVCEAGSGLVFSCQ DKQNTVCEECPDGTYSDEANHVDPCLPCTVCEDTERQLRECTRWADAECEEIPGRWITR STPPEGSDSTAPSTQEPEAPPEQDLIASTVAGVVTTVMGSSQPVVTRGTTDN (SEQ ID NO: 26), preferably ΔCD34.

8. The chimeric antigen receptor according to claim 1, wherein the hinge CD8stalk is linked to the trackable marker ΔCD34.

9. The chimeric antigen receptor according to claim 1, wherein the trans membrane domain is chosen from the group consisting of CD28TM FWVLVVVGGVLACYSLLVTVAFIIFWV (SEQ ID NO:27) or CD8aTM IYIWAPLAGTCGVLLLSLVIT (SEQ ID NO:28), preferably CD8aTM.

10. The chimeric antigen receptor according to claim 1, wherein the co-stimulatory signaling domain is chosen from the group consisting of
CD28 cytoplasmic sequence RSKRSRLLHSDYMNMTPRRPGPTRKHYQPYAPPRDFAAYRS (SEQ ID NO:29),
CD137 (4-1BB) sequence KRGRKKLLYIFKQPFMRPVQTTQEEDGCSCRFPEEEEGGCEL (SEQ ID NO:30),
OX40 sequence RDQRLPPDAHKPPGGGSFRTPIQEEQADAHSTLAKI (SEQ ID NO: 31), or
a sequence obtained by linking:
CD28 cytoplasmic sequence (SEQ ID NO:29) with CD137 (4-1BB) sequence (SEQ ID NO: 30),
CD137 (4-1BB) sequence (SEQ ID NO:30) with CD28 cytoplasmic sequence (SEQ ID NO: 29),
CD28 cytoplasmic sequence (SEQ ID NO:29) with OX40 sequence (SEQ ID NO:31),
OX40 sequence (SEQ ID NO:31) with CD28 cytoplasmic sequence (SEQ ID NO:29),
OX40 sequence (SEQ ID NO:31) with CD137 (4-1BB) sequence (SEQ ID NO:30),
CD137 (4-1BB) sequence (SEQ ID NO:30) with OX40 sequence (SEQ ID NO:31)

11. The chimeric antigen receptor according to claim 1, wherein CD3Zeta chain sequence is RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR* (SEQ ID NO:32).

12. The chimeric antigen receptor according to claim 1, further comprising cytoplasmic moiety of CD8cyt: LYCNHRNRRRVCKCPR (SEQ ID NO:40) between the transmembrane domain and the co-stimulatory signaling domain.

13. The chimeric antigen receptor according to claim 1, wherein the signal peptide comprises or consists of MEFGLSWLFLVAILKGVQC (SEQ ID NO:41).

14. The chimeric antigen receptor according to claim 1, wherein said anti-CD19 chimeric antigen receptor comprises or consists of the following sequence:
(SEQ ID NO: 72)
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGSGGGGEVKLQESGPGLVAP
SQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGT
SVTVSSGSELPTQGTFSNVSTNVSPAPRPPTPAPTIASQPLSLRPEACRP
AAGGAVHTRGLDFACDIYIWAPLAGTCGVLLLSLVITLYCNHRNRRRVCK
CPRVDKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR
or
(SEQ ID NO: 33)
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGSGGGGEVKLQESGPGLVAP
SQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGT
SVTVSSGSPAPRPPTPAPTIASQPLSLRPEACRPAAGGAVHTRGLDFACD
IYIWAPLAGTCGVLLLSLVITLYCNHRNRRRVCKCPRVDKRGRKKLLYIF
KQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPR.

15. Nucleotide A nucleotide sequence comprising or consisting of a nucleotide sequence which encodes a sequence encoding the chimeric antigen receptor according to claim 1.

16. Nucleotide The nucleotide sequence according to claim 15. wherein:
anti CD19 FMC63 hybridoma VL sequence is encoded by the nucleotide sequence
GACATCCAGATGACACAGACTACATCCTCCCTGTCTGCCTCTCTGGGAGACA GAGTCACCATCAGTTGCAGGGCAAGTCAGGACATTAGTAAATATTTAAATTGGTATC AGCAGAAACCAGATGGAACTGTTAAACTCCTGATCTACCATACATCAAGATTACACT CAGGAGTCCCATCAAGGTTCAGTGGCAGTGGGTCTGGAACAGATTATTCTCTCACCA TTAGCAACCTGGAGCAAGAAGATATTGCCACTTACTTTTGCCAACAGGGTAATACGC TTCCGTACACGTTCGGAGGGGGGACTAAGTTGGAAATAACA (SEQ ID NO:52) and
anti CD19 FMC63 hybridoma VH sequence is encoded by the nucleotide sequence
GAGGTGAAACTGCAGGAGTCAGGACCTGGCCTGGTGGCGCCCTCACAGAGCC TGTCCGTCACATGCACTGTCTCAGGGGTCTCATTACCCGACTATGGTGTAAGCTGGA TTCGCCAGCCTCCACGAAAGGGTCTGGAGTGGCTGGGAGTAATATGGGGTAGTGAA ACCACATACTATAATTCAGCTCTCAAATCCAGACTGACCATCATCAAGGACAACTCC AAGAGCCAAGTTTTCTTAAAAATGAACAGTCTGCAAACTGATGACACAGCCATTTAC TACTGTGCCAAACATTATTACTACGGTGGTAGCTATGCTATGGACTACTGGGGTCAA GGAACCTCAGTCACCGTCTCCTCA (SEQ ID NO:53);
anti CD20 VL sequence is encoded by the nucleotide sequence
CAGATCGTGCTGAGCCAGAGCCCCGCCATCCTGAGCGCCAGCCCCGGCGAGA AGGTGACCATGACCTGCAGGGCCAGCAGCAGCGTGAGCTACATCCACTGGTTCCAG CAGAAGCCCGGCAGCAGCCCCAAGCCCTGGATCTACGCCACCAGCAACCTGGCCAG CGGCGTGCCCGTGAGGTTCAGCGGCAGCGGCAGCGGCACCAGCTACAGCCTGACCA TCAGCAGGGTGGAGGCCGAGGACGCCGCCACCTACTACTGCCAGCAGTGGACCAGC AACCCCCCCACCTTCGGCGGCGGCACCAAGCTGGAGATCAAG (SEQ ID NO:54) and
anti CD20 VH sequence is encoded by the nucleotide sequence
CAGGTGCAGCTGCAGCAGCCCGGCGCCGAGCTGGTGAAGCCCGGCGCCAGC GTGAAGATGAGCTGCAAGGCCAGCGGCTACACCTTCACCAGCTACAACATGCACTG GGTGAAGCAGACCCCCGGCAGGGGCCTGGAGTGGATCGGCGCCATCTACCCCGGCA ACGGCGACACCAGCTACAACCAGAAGTTCAAGGGCAAGGCCACCCTGACCGCCGAC AAGAGCAGCAGCACCGCCTACATGCAGCTGAGCAGCCTGACCAGCGAGGACAGCGC CGTGTACTACTGCGCCAGGAGCACCTACTACGGCGGCGACTGGTACTTCAACGTGTG GGGCGCCGGCACCACCGTGACCGTGAGC (SEQ ID NO:55);
anti CD22 VL sequence is encoded by the nucleotide sequence
GACGTGCAGGTGACCCAGAGCCCCAGCAGCCTGAGCGCCAGCGTGGGCGAC AGGGTGACCATCACCTGCAGGAGCAGCCAGAGCCTGGCCAACAGCTACGGCAACAC CTTCCTGAGCTGGTACCTGCACAAGCCCGGCAAGGCCCCCCAGCTGCTGATCTACGG CATCAGCAACAGGTTCAGCGGCGTGCCCGACAGGTTCAGCGGCAGCGGCAGCGGCA CCGACTTCACCCTGACCATCAGCAGCCTGCAGCCCGAGGACTTCGCCACCTACTACT GCCTGCAGGGCACCCACCAGCCCTACACCTTCGGCCAGGGCACCAAGGTGGAGATC AAG (SEQ ID NO:56) and
anti CD22 VH sequence is encoded by the nucleotide sequence
GAGGTGCAGCTGGTGCAGAGCGGCGCCGAGGTGAAGAAGCCCGGCGCCAGC GTGAAGGTGAGCTGCAAGGCCAGCGGCTACAGGTTCACCAACTACTGGATCCACTG GGTGAGGCAGGCCCCCGGCCAGGGCCTGGAGTGGATCGGCGGCATCAACCCCGGCA ACAACTACGCCACCTACAGGAGGAAGTTCCAGGGCAGGGTGACCATGACCGCCGAC ACCAGCACCAGCACCGTGTACATGGAGCTGAGCAGCCTGAGGAGCGAGGACACCGC CGTGTACTACTGCACCAGGGAGGGCTACGGCAACTACGGCGCCTGGTTCGCCTACTG GGGCCAGGGCACCCTGGTGACCGTGAGCAGC (SEQ ID NO:57).

17. The nucleotide sequence according to claim 15, wherein the nucleotide sequence encoding anti-CD19 chimeric antigen receptor is:
(SEQ ID NO: 58)
ATGGAGTTTGGACTTTCTTGGTTGTTTTTGGTGGCAATTCTGAAGGGTGT
CCAGTGTAGCAGGGACATCCAGATGACACAGACTACATCCTCCCTGTCTG
CCTCTCTGGGAGACAGAGTCACCATCAGTTGCAGGGCAAGTCAGGACATT
AGTAAATATTTAAATTGGTATCAGCAGAAACCAGATGGAACTGTTAAACT
CCTGATCTACCATACATCAAGATTACACTCAGGAGTCCCATCAAGGTTCA
GTGGCAGTGGGTCTGGAACAGATTATTCTCTCACCATTAGCAACCTGGAG
CAAGAAGATATTGCCACTTACTTTTGCCAACAGGGTAATACGCTTCCGTA
CACGTTCGGAGGGGGGACTAAGTTGGAAATAACAGGCGGAGGAAGCGGAG
GTGGGGGCGAGGTGAAACTGCAGGAGTCAGGACCTGGCCTGGTGGCGCCC
TCACAGAGCCTGTCCGTCACATGCACTGTCTCAGGGGTCTCATTACCCGA
CTATGGTGTAAGCTGGATTCGCCAGCCTCCACGAAAGGGTCTGGAGTGGC
TGGGAGTAATATGGGGTAGTGAAACCACATACTATAATTCAGCTCTCAAA
TCCAGACTGACCATCATCAAGGACAACTCCAAGAGCCAAGTTTTCTTAAA
AATGAACAGTCTGCAAACTGATGACACAGCCATTTACTACTGTGCCAAAC
ATTATTACTACGGTGGTAGCTATGCTATGGACTACTGGGGTCAAGGAACC
TCAGTCACCGTCTCCTCAGGATCCGAACTTCCTACTCAGGGGACTTTCTC
AAACGTTAGCACAAACGTAAGTCCCGCCCCAAGACCCCCCACACCTGCGC
CGACCATTGCTTCTCAACCCCTGAGTTTGAGACCCGAGGCCTGCCGGCCA
GCTGCCGGCGGGGCCGTGCATACAAGAGGACTCGATTTCGCTTGCGACAT
CTATATCTGGGCACCTCTCGCTGGCACCTGTGGAGTCCTTCTGCTCAGCC
TGGTTATTACTCTGTACTGTAATCACCGGAATCGCCGCCGCGTTTGTAAG
TGTCCCAGGGTCGACAAACGGGGCAGAAAGAAACTCCTGTATATATTCAA
ACAACCATTTATGAGACCAGTACAAACTACTCAAGAGGAAGATGGCTGTA
GCTGCCGATTTCCAGAAGAAGAAGAAGGAGGATGTGAACTGAGAGTGAAG
TTCAGCAGGAGCGCAGACGCCCCCGCGTACCAGCAGGGCCAGAACCAGCT
CTATAACGAGCTCAATCTAGGACGAAGAGAGGAGTACGATGTTTTGGACA
AGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGC
CTACAGTGAGATTGGGATGAAAGGCGAGCGCCGGAGGGGCAAGGGGCACG
ATGGCCTTTACCAGGGTCTCAGTACAGCCACCAAGGACACCTACGACGCC
CTTCACATGCAGGCCCTGCCCCCTCGCTAA
or
(SEQ ID NO: 34)
ATGGAGTTTGGACTTTCTTGGTTGTTTTTGGTGGCAATTCTGAAGGGTGT
CCAGTGTAGCAGGGACATCCAGATGACACAGACTACATCCTCCCTGTCTG
CCTCTCTGGGAGACAGAGTCACCATCAGTTGCAGGGCAAGTCAGGACATT
AGTAAATATTTAAATTGGTATCAGCAGAAACCAGATGGAACTGTTAAACT
CCTGATCTACCATACATCAAGATTACACTCAGGAGTCCCATCAAGGTTCA
GTGGCAGTGGGTCTGGAACAGATTATTCTCTCACCATTAGCAACCTGGAG
CAAGAAGATATTGCCACTTACTTTTGCCAACAGGGTAATACGCTTCCGTA
CACGTTCGGAGGGGGGACTAAGTTGGAAATAACAGGCGGAGGAAGCGGAG
GTGGGGGCGAGGTGAAACTGCAGGAGTCAGGACCTGGCCTGGTGGCGCCC
TCACAGAGCCTGTCCGTCACATGCACTGTCTCAGGGGTCTCATTACCCGA
CTATGGTGTAAGCTGGATTCGCCAGCCTCCACGAAAGGGTCTGGAGTGGC
TGGGAGTAATATGGGGTAGTGAAACCACATACTATAATTCAGCTCTCAAA
TCCAGACTGACCATCATCAAGGACAACTCCAAGAGCCAAGTTTTCTTAAA
AATGAACAGTCTGCAAACTGATGACACAGCCATTTACTACTGTGCCAAAC
ATTATTACTACGGTGGTAGCTATGCTATGGACTACTGGGGTCAAGGAACC
TCAGTCACCGTCTCCTCAGGATCCCCCGCCCCAAGACCCCCCACACCTGC
GCCGACCATTGCTTCTCAACCCCTGAGTTTGAGACCCGAGGCCTGCCGGC
CAGCTGCCGGCGGGGCCGTGCATACAAGAGGACTCGATTTCGCTTGCGAC
ATCTATATCTGGGCACCTCTCGCTGGCACCTGTGGAGTCCTTCTGCTCAG
CCTGGTTATTACTCTGTACTGTAATCACCGGAATCGCCGCCGCGTTTGTA
AGTGTCCCAGGGTCGACAAACGGGGCAGAAAGAAACTCCTGTATATATTC
AAACAACCATTTATGAGACCAGTACAAACTACTCAAGAGGAAGATGGCTG
TAGCTGCCGATTTCCAGAAGAAGAAGAAGGAGGATGTGAACTGAGAGTGA
AGTTCAGCAGGAGCGCAGACGCCCCCGCGTACCAGCAGGGCCAGAACCAG
CTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGA
ACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCA
CGATGGCCTTTACCAGGGTCTCAGTACAGCCACCAAGGACACCTACGACG
CCCTTCACATGCAGGCCCTGCCCCCTCGCTAAA.

18. The nucleotide sequence according to claim 15, said nucleotide sequence further comprising a nucleotide sequence encoding a suicide gene inducible amino acid sequence linked to the nucleotide sequence encoding said chimeric antigen receptor by a nucleotide sequence encoding a 2A self-cleaving peptide.

19. The nucleotide sequence according to claim 18, wherein the suicide gene inducible amino acid sequence is a chimeric Caspase-9 polypeptide or comprises a herpes simplex virus thymidine kinase.

20. The nucleotide sequence according to claim 15, which is
(SEQ ID NO: 180)
ATGCTCGAGATGCTGGAGGGAGTGCAGGTGGAGACTATTAGCCCCGGAGA
TGGCAGAACATTCCCCAAAAGAGGACAGACTTGCGTCGTGCATTATACTG
GAATGCTGGAAGACGGCAAGAAGGTGGACAGCAGCCGGGACCGAAACAAG
CCCTTCAAGTTCATGCTGGGGAAGCAGGAAGTGATCCGGGGCTGGGAGGA
AGGAGTCGCACAGATGTCAGTGGGACAGAGGGCCAAACTGACTATTAGCC
CAGACTACGCTTATGGAGCAACCGGCCACCCCGGGATCATTCCCCCTCAT
GCTACACTGGTCTTCGATGTGGAGCTGCTGAAGCTGGAAAGCGGAGGAGG
ATCCGGAGTGGACGGGTTTGGAGATGTGGGAGCCCTGGAATCCCTGCGGG
GCAATGCCGATCTGGCTTACATCCTGTCTATGGAGCCTTGCGGCCACTGT
CTGATCATTAACAATGTGAACTTCTGCAGAGAGAGCGGGCTGCGGACCAG
AACAGGATCCAATATTGACTGTGAAAAGCTGCGGAGAAGGTTCTCTAGTC
TGCACTTTATGGTCGAGGTGAAAGGCGATCTGACCGCTAAGAAAATGGTG
CTGGCCCTGCTGGAACTGGCTCGGCAGGACCATGGGGCACTGGATTGCTG
CGTGGTCGTGATCCTGAGTCACGGCTGCCAGGCTTCACATCTGCAGTTCC
CTGGGGCAGTCTATGGAACTGACGGCTGTCCAGTCAGCGTGGAGAAGATC
GTGAACATCTTCAACGGCACCTCTTGCCCAAGTCTGGGCGGGAAGCCCAA
ACTGTTCTTTATTCAGGCCTGTGGAGGCGAGCAGAAAGATCACGGCTTCG
AAGTGGCTAGCACCTCCCCCGAGGACGAATCACCTGGAAGCAACCCTGAG
CCAGATGCAACCCCCTTCCAGGAAGGCCTGAGGACATTTGACCAGCTGGA
TGCCATCTCAAGCCTGCCCACACCTTCTGACATTTTCGTCTCTTACAGTA
CTTTCCCTGGATTTGTGAGCTGGCGCGATCCAAAGTCAGGCAGCTGGTAC
GTGGAGACACTGGACGATATCTTTGAGCAGTGGGCCCATTCTGAAGACCT
GCAGAGTCTGCTGCTGCGAGTGGCCAATGCTGTCTCTGTGAAGGGGATCT
ACAAACAGATGCCAGGATGCTTCAACTTTCTGAGAAAGAAACTGTTCTTT
AAGACCTCCGCATCTAGGGCCCCGCGGGAAGGCCGAGGGAGCCTGCTGAC
ATGTGGCGATGTGGAGGAAAACCCAGGACCACCATGGATGGAGTTTGGAC
TTTCTTGGTTGTTTTTGGTGGCAATTCTGAAGGGTGTCCAGTGTAGCAGG
GACATCCAGATGACACAGACTACATCCTCCCTGTCTGCCTCTCTGGGAGA
CAGAGTCACCATCAGTTGCAGGGCAAGTCAGGACATTAGTAAATATTTAA
ATTGGTATCAGCAGAAACCAGATGGAACTGTTAAACTCCTGATCTACCAT
ACATCAAGATTACACTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGGTAATACGCTTCCGTACACGTTCGGAGGG
GGGACTAAGTTGGAAATAACAGGCGGAGGAAGCGGAGGTGGGGGCGAGGT
GAAACTGCAGGAGTCAGGACCTGGCCTGGTGGCGCCCTCACAGAGCCTGT
CCGTCACATGCACTGTCTCAGGGGTCTCATTACCCGACTATGGTGTAAGC
TGGATTCGCCAGCCTCCACGAAAGGGTCTGGAGTGGCTGGGAGTAATATG
GGGTAGTGAAACCACATACTATAATTCAGCTCTCAAATCCAGACTGACCA
TCATCAAGGACAACTCCAAGAGCCAAGTTTTCTTAAAAATGAACAGTCTG
CAAACTGATGACACAGCCATTTACTACTGTGCCAAACATTATTACTACGG
TGGTAGCTATGCTATGGACTACTGGGGTCAAGGAACCTCAGTCACCGTCT
CCTCAGGATCCGAACTTCCTACTCAGGGGACTTTCTCAAACGTTAGCACA
AACGTAAGTCCCGCCCCAAGACCCCCCACACCTGCGCCGACCATTGCTTC
TCAACCCCTGAGTTTGAGACCCGAGGCCTGCCGGCCAGCTGCCGGCGGGG
CCGTGCATACAAGAGGACTCGATTTCGCTTGCGACATCTATATCTGGGCA
CCTCTCGCTGGCACCTGTGGAGTCCTTCTGCTCAGCCTGGTTATTACTCT
GTACTGTAATCACCGGAATCGCCGCCGCGTTTGTAAGTGTCCCAGGGTCG
ACAAACGGGGCAGAAAGAAACTCCTGTATATATTCAAACAACCATTTATG
AGACCAGTACAAACTACTCAAGAGGAAGATGGCTGTAGCTGCCGATTTCC
AGAAGAAGAAGAAGGAGGATGTGAACTGAGAGTGAAGTTCAGCAGGAGCG
CAGACGCCCCCGCGTACCAGCAGGGCCAGAACCAGCTCTATAACGAGCTC
AATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCC
TGTACAATGAACTGCAGAAAGATAAGATGGCGGAGGCCTACAGTGAGATT
GGGATGAAAGGCGAGCGCCGGAGGGGCAAGGGGCACGATGGCCTTTACCA
GGGTCTCAGTACAGCCACCAAGGACACCTACGACGCCCTTCACATGCAGG
CCCTGCCCCCTCGCTAA.

21. A vector comprising the nucleotide sequence according to claim 15, wherein said vector is a DNA vector, a RNA vector, a plasmid, a lentivirus vector, adenoviral vector, retrovirus vector, or non-viral vector.

22. A cell comprising the chimeric antigen receptor according to claim 1, wherein the cell is an alfa/beta and gamma/delta T cell, NK cell, and/or NK-T cell.

23. The cell according to claim 22, further comprising a suicide gene inducible amino acid sequence a herpes simplex virus thymidine kinase.

24. The cell according to claim 23, wherein the chimeric Caspase-9 polypeptide comprises:
FKBP12 binding region comprising or consisting of a short 5′ leader peptide MLEMLE (SEQ ID NO:43) and the mutant of human FKBP12 (V36F) of sequence
GVQVETISPGDGRTFPKRGQTCVVHYTGMLEDGKKVDSSRDRNKPFKFMLGKQ EVIRGWEEGVAQMSVGQRAKLTISPDYAYGATGHPGIIPPHATLVFDVELLKLE (SEQ ID NO: 44), which is linked by a linker, such as SGGGSG (SEQ ID NO:45) linker, to Caspase-9 polypeptide
VDGFGDVGALESLRGNADLAYILSMEPCGHCLIINNVNFCRESGLRTRTGSNIDC EKLRRRFSSLHFMVEVKGDLTAKKMVLALLELARQDHGALDCCVVVILSHGCQASHLQ FPGAVYGTDGCPVSVEKIVNIFNGTSCPSLGGKPKLFFIQACGGEQKDHGFEVASTSPED ESPGSNPEPDATPFQEGLRTFDQLDAISSLPTPSDIFVSYSTFPGFVSWRDPKSGSWYVET LDDIFEQWAHSEDLQSLLLRVANAVSVKGIYKQMPGCFNFLRKKLFFKTS (SEQ ID NO: 46), which is linked by a linker, such as ASRAPR (SEQ ID NO:47) linker, to
a Polynucleotide 2A self-cleaving peptide chosen from the group consisting of T2A AEGRGSLLTCGDVEENPGP (SEQ ID NO:48), P2A ATNFSLLKQAGDVEENPGP (SEQ ID NO: 49), E2A QCTNYALLKLAGDVESNPGP (SEQ ID NO:50) or F2A: VKQTLNFDLLKLAGDVESNPGP (SEQ ID NO:51), preferably T2A.

25. The cell according to 22, which is obtained in culture conditions comprising an activation step, transduction step and/or expansion step of the process for the preparation of said cell in the presence of both IL-7 and IL-15.

26. A pharmaceutical composition comprising the cell according to claim 22 together with one or more excipients and/or adjuvants.

27. (canceled)

28. A method for treatment of CD19+, CD20+ or CD22+ cancers, B cell lymphomas (Non-Hodgkin's Lymphoma (NHL)), acute lymphoblastic leukemia (ALL), myeloid leukemia and chronic lymphocytic leukemia (CLL), B-cell derived autoimmune diseases, the method comprising:
identifying a subject in need thereof;
administering to the subject the vector according to claim 21.

29. A method for treatment of CD19+, CD20+ or CD22+ cancers, B cell lymphomas (Non-Hodgkin's Lymphoma (NHL)), acute lymphoblastic leukemia (ALL), myeloid leukemia and chronic lymphocytic leukemia (CLL), B-cell derived autoimmune diseases, the method comprising:
identifying a subject in need thereof;
administering to the subject the cell according to claim 22.

30. A method for treatment of CD19+, CD20+ or CD22+ cancers, B cell lymphomas (Non-Hodgkin's Lymphoma (NHL)), acute lymphoblastic leukemia (ALL), myeloid leukemia and chronic lymphocytic leukemia (CLL), B-cell derived autoimmune diseases, the method comprising:
identifying a subject in need thereof;
administering to the subject the pharmaceutical composition according to claim 26.


SEQUENCE LISTING (191 sequences)

SEQ ID NO: 1 - protein, 6 aa, Homo sapiens
QDISKY

SEQ ID NO: 2 - protein, 5 aa, Homo sapiens
GNTLP

SEQ ID NO: 3 - protein, 8 aa, Homo sapiens
GVSLPDYG

SEQ ID NO: 4 - protein, 7 aa, Homo sapiens
IWGSETT

SEQ ID NO: 5 - protein, 14 aa, Homo sapiens
AKHYYYGGSYAMDY

SEQ ID NO: 6 - protein, 5 aa, Artificial Sequence
  CDR1 of anti CD20  VL sequence
SSVSY

SEQ ID NO: 7 - protein, 9 aa, Artificial Sequence
  CDR3 of anti CD20 VL sequence
QQWTSNPPT

SEQ ID NO: 8 - protein, 8 aa, Artificial Sequence
  CDR1 of anti CD20 VH sequence
GYTFTSYN

SEQ ID NO: 9 - protein, 8 aa, Artificial Sequence
  CDR2 of anti CD20 VH sequence
IYPGNGDT

SEQ ID NO: 10 - protein, 14 aa, Artificial Sequence
  CDR3 of antiCD20 VH sequence
ARSTYYGGDWYFNV

SEQ ID NO: 11 - protein, 11 aa, Artificial Sequence
  CDR1 od antiCD22 VL sequence
QSLANSYGNTF

SEQ ID NO: 12 - protein, 7 aa, Artificial Sequence
  CDR3 of antiCD22 VL sequence
LQGTHQP

SEQ ID NO: 13 - protein, 10 aa, Artificial Sequence
  CDR1 of antiCD22 VH sequence
GYRFTNYWIH

SEQ ID NO: 14 - protein, 8 aa, Artificial Sequence
  CDR2 of antiCD22 VH sequence
INPGNNYA

SEQ ID NO: 15 - protein, 107 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 16 - protein, 120 aa, Homo sapiens
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 17 - protein, 106 aa, Homo sapiens [sequence omitted]

SEQ ID NO: 18 - protein, 121 aa, Artificial Sequence [sequence omitted]

SEQ ID NO: 19 - protein, 112 aa, Artificial Sequence
  anti CD22 VL sequence
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIK

SEQ ID NO: 20 - protein, 121 aa, Artificial Sequence
  anti CD22 VH sequence
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 21 - protein, 45 aa, Artificial Sequence
  CD8stalk
TTTPAPRPPTPAPTIASQPLSLRPEACRPAAGGAVHTRGLDFACD

SEQ ID NO: 22 - protein, 38 aa, Artificial Sequence
  Hinge CD28
EVMYPPPYLDNEKSNGTIIHVKGKHLCPSPLFPGPSKP

SEQ ID NO: 23 - protein, 229 aa, Artificial Sequence
  hinge CH2-CH3
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 119 aa, Artificial Sequence
  hinge CH3
ESKYGPPCPSCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 16 aa, Artificial Sequence
  ?CD34
ELPTQGTFSNVSTNVS

SEQ ID NO: 26 - protein, 222 aa, Artificial Sequence
  NGFR
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDN

SEQ ID NO: 27 - protein, 27 aa, Artificial Sequence
  CD28TM
FWVLVVVGGVLACYSLLVTVAFIIFWV

SEQ ID NO: 28 - protein, 21 aa, Artificial Sequence
  CD8aTM
IYIWAPLAGTCGVLLLSLVIT

SEQ ID NO: 29 - protein, 41 aa, Artificial Sequence
  CD28 cytoplasmic sequence
RSKRSRLLHSDYMNMTPRRPGPTRKHYQPYAPPRDFAAYRS

SEQ ID NO: 30 - protein, 42 aa, Artificial Sequence
  CD137 sequence
KRGRKKLLYIFKQPFMRPVQTTQEEDGCSCRFPEEEEGGCEL

SEQ ID NO: 31 - protein, 36 aa, Artificial Sequence
  OX40 sequence
RDQRLPPDAHKPPGGGSFRTPIQEEQADAHSTLAKI

SEQ ID NO: 32 - protein, 112 aa, Artificial Sequence
  CD3Zeta chain sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 493 aa, Artificial Sequence
  anti-CD19 chimeric antigen receptor
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSPAPRPPTPAPTIASQPLSLRPEACRPAAGGAVHTRGLDFACDIYIWAPLAGTCGVLLLSLVITLYCNHRNRRRVCKCPRVDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1483 bp, Artificial Sequence
  Nucleotide sequence encoding SEQ ID NO33
atggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggcggaggaagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatcccccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaa

SEQ ID NO: 35 - protein, 9 aa, Artificial Sequence
  linker
GGGGSGGGG

SEQ ID NO: 36 - DNA, 1566 bp, Artificial Sequence
  Nucleotide sequence encoding SEQ ID NO39
atggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaagcggaggtgggggcagcggaggtgggggcagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccgcatgcgaacttcctactcaggggactttctcaaacgttagcacaaacgtaagtgcggccgcccccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 37 - protein, 7 aa, Artificial Sequence
  linker
GGGGSGG

SEQ ID NO: 38 - protein, 8 aa, Artificial Sequence
  linker
GGGSGGGG

SEQ ID NO: 39 - protein, 521 aa, Artificial Sequence
  anti-CD19 chimeric antigen receptor
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGGGGSGGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSACELPTQGTFSNVSTNVSAAAPAPRPPTPAPTIASQPLSLRPEACRPAAGGAVHTRGLDFACDIYIWAPLAGTCGVLLLSLVITLYCNHRNRRRVCKCPRVDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 16 aa, Artificial Sequence
  cytoplasmic moiety of CD8cyt
LYCNHRNRRRVCKCPR

SEQ ID NO: 41 - protein, 19 aa, Artificial Sequence
  signal peptide
MEFGLSWLFLVAILKGVQC

SEQ ID NO: 42 - protein, 5 aa, herpes simplex virus
HSVTK

SEQ ID NO: 43 - protein, 6 aa, Artificial Sequence
  short 5′ leader peptide
MLEMLE

SEQ ID NO: 44 - protein, 107 aa, Artificial Sequence
  mutant sequence of human FKBP12(V36F)
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 45 - protein, 6 aa, Artificial Sequence
  linker
SGGGSG

SEQ ID NO: 46 - protein, 284 aa, Artificial Sequence
  Caspase-9 polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 47 - protein, 6 aa, Artificial Sequence
  linker
ASRAPR

SEQ ID NO: 48 - protein, 19 aa, Thosea asigna virus 2
AEGRGSLLTCGDVEENPGP

SEQ ID NO: 49 - protein, 19 aa, porcine teschovirus-1
ATNFSLLKQAGDVEENPGP

SEQ ID NO: 50 - protein, 20 aa, equine rhinitis A virus
QCTNYALLKLAGDVESNPGP

SEQ ID NO: 51 - protein, 22 aa, foot-and-mouth disease virus
VKQTLNFDLLKLAGDVESNPGP

SEQ ID NO: 52 - DNA, 321 bp, Artificial Sequence
  Nucleotide sequence encoding SEQ ID NO15
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 53 - DNA, 360 bp, Artificial Sequence
  Nucleotide sequence encoding SEQ ID NO16
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 54 - DNA, 318 bp, Artificial Sequence
  Nucleotide sequence encoding SEQ ID NO17
cagatcgtgctgagccagagccccgccatcctgagcgccagccccggcgagaaggtgaccatgacctgcagggccagcagcagcgtgagctacatccactggttccagcagaagcccggcagcagccccaagccctggatctacgccaccagcaacctggccagcggcgtgcccgtgaggttcagcggcagcggcagcggcaccagctacagcctgaccatcagcagggtggaggccgaggacgccgccacctactactgccagcagtggaccagcaacccccccaccttcggcggcggcaccaagctggagatcaag

SEQ ID NO: 55 - DNA, 360 bp, Artificial Sequence
  Nucleotide sequence encoding SEQ ID NO18
caggtgcagctgcagcagcccggcgccgagctggtgaagcccggcgccagcgtgaagatgagctgcaaggccagcggctacaccttcaccagctacaacatgcactgggtgaagcagacccccggcaggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgccaggagcacctactacggcggcgactggtacttcaacgtgtggggcgccggcaccaccgtgaccgtgagc

SEQ ID NO: 56 - DNA, 336 bp, Artificial Sequence
  Nucleotide sequence encoding SEQ ID NO19
gacgtgcaggtgacccagagccccagcagcctgagcgccagcgtgggcgacagggtgaccatcacctgcaggagcagccagagcctggccaacagctacggcaacaccttcctgagctggtacctgcacaagcccggcaaggccccccagctgctgatctacggcatcagcaacaggttcagcggcgtgcccgacaggttcagcggcagcggcagcggcaccgacttcaccctgaccatcagcagcctgcagcccgaggacttcgccacctactactgcctgcagggcacccaccagccctacaccttcggccagggcaccaaggtggagatcaag

SEQ ID NO: 57 - DNA, 363 bp, Artificial Sequence
  Nucleotide sequence encoding SEQ ID NO20
gaggtgcagctggtgcagagcggcgccgaggtgaagaagcccggcgccagcgtgaaggtgagctgcaaggccagcggctacaggttcaccaactactggatccactgggtgaggcaggcccccggccagggcctggagtggatcggcggcatcaaccccggcaacaactacgccacctacaggaggaagttccagggcagggtgaccatgaccgccgacaccagcaccagcaccgtgtacatggagctgagcagcctgaggagcgaggacaccgccgtgtactactgcaccagggagggctacggcaactacggcgcctggttcgcctactggggccagggcaccctggtgaccgtgagcagc

SEQ ID NO: 58 - DNA, 1530 bp, Artificial Sequence
  Nucleotide sequence encoding SEQ ID NO72
atggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggcggaggaagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 59 - DNA, 339 bp, Artificial Sequence
  Nucleotide sequence encoding SEQ ID NO44
atgctcgagatgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaa

SEQ ID NO: 60 - DNA, 18 bp, Artificial Sequence
  Nucleotide sequence encoding SEQ ID NO45
agcggaggaggatccgga

SEQ ID NO: 61 - DNA, 852 bp, Artificial Sequence
  Nucleotide sequence encoding SEQ ID NO46
gtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctcc

SEQ ID NO: 62 - DNA, 18 bp, Artificial Sequence
  Nucleotide sequence encoding SEQ ID NO47
gcatctagggccccgcgg

SEQ ID NO: 63 - DNA, 54 bp, Artificial Sequence
  Nucleotide sequence encoding SEQ ID NO48
gaaggccgagggagcctgctgacatgtggcgatgtggaggaaaacccaggacca

SEQ ID NO: 64 - DNA, 63 bp, Artificial Sequence
  Nucleotide sequence encoding SEQ ID NO41
atggagtttggactttcttggttgtttttggtggcaattctgaagggtgtccagtgtagcagg

SEQ ID NO: 65 - DNA, 24 bp, Artificial Sequence
  Nucleotide sequence encoding SEQ ID NO38
ggcggaggaagcggaggtgggggc

SEQ ID NO: 66 - DNA, 48 bp, Artificial Sequence
  Nucleotide sequence encoding SEQ ID NO25
gaacttcctactcaggggactttctcaaacgttagcacaaacgtaagt

SEQ ID NO: 67 - DNA, 126 bp, Artificial Sequence
  Nucleotide sequence encoding SEQ ID NO21
cccgccccaagaccccccacacctgcgccgaccattgcttctcaacccctgagtttgagacccgaggcctgccggccagctgccggcggggccgtgcatacaagaggactcgatttcgcttgcgac

SEQ ID NO: 68 - DNA, 63 bp, Artificial Sequence
  Nucleotide sequence encoding SEQ ID NO28
atctatatctgggcacctctcgctggcacctgtggagtccttctgctcagcctggttattact

SEQ ID NO: 69 - DNA, 48 bp, Artificial Sequence
  Nucleotide sequence encoding SEQ ID NO40
ctgtactgtaatcaccggaatcgccgccgcgtttgtaagtgtcccagg

SEQ ID NO: 70 - DNA, 126 bp, Artificial Sequence
  Nucleotide sequence encoding SEQ ID NO30
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 71 - DNA, 339 bp, Artificial Sequence
  Nucleotide sequence encoding SEQ ID NO32
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 72 - protein, 509 aa, Artificial Sequence
  anti-CD19 chimeric antigen receptor
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - DNA, 1503 bp, Artificial Sequence
  Nucleotide sequence encoding SEQ ID NO74
atggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaagcggaggtgggggcagcggaggtgggggcagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatcccccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 74 - protein, 500 aa, Artificial Sequence
  anti-CD19 chimeric antigen receptor
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGGGGSGGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSPAPRPPTPAPTIASQPLSLRPEACRPAAGGAVHTRGLDFACDIYIWAPLAGTCGVLLLSLVITLYCNHRNRRRVCKCPRVDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 24 bp, Artificial Sequence
  primer
gtccgcaatttttcattggtagta

SEQ ID NO: 76 - DNA, 30 bp, Artificial Sequence
  primer
gcaagctacacaattaaaggagaagatagt

SEQ ID NO: 77 - DNA, 27 bp, Artificial Sequence
  primer
tagagatccggccttttaactggaact

SEQ ID NO: 78 - DNA, 18 bp, Artificial Sequence
  primer
gcagcaccccctcaagca

SEQ ID NO: 79 - DNA, 39 bp, Artificial Sequence
  primer
tgtgcgaaagatctttttttatggtgtatgctatttctt

SEQ ID NO: 80 - DNA, 17 bp, Artificial Sequence
  primer
cgtatcccccccccaca

SEQ ID NO: 81 - DNA, 23 bp, Artificial Sequence
  primer
gccccggactagctagtttacga

SEQ ID NO: 82 - DNA, 24 bp, Artificial Sequence
  primer
actgtccccgaggttgtactaatg

SEQ ID NO: 83 - DNA, 18 bp, Artificial Sequence
  primer
tgctataccgggcgggtg

SEQ ID NO: 84 - DNA, 21 bp, Artificial Sequence
  primer
cccagtaaggtcggtggagtc

SEQ ID NO: 85 - DNA, 20 bp, Artificial Sequence
  primer
cgtatcccccaggagaagca

SEQ ID NO: 86 - DNA, 30 bp, Artificial Sequence
  primer
atagatgtgtactactgtgcgagcgtacta

SEQ ID NO: 87 - DNA, 26 bp, Artificial Sequence
  primer
tccggttggtatcacctatcccctaa

SEQ ID NO: 88 - DNA, 21 bp, Artificial Sequence
  primer
tgtgcgtatcccccagagaca

SEQ ID NO: 89 - DNA, 27 bp, Artificial Sequence
  primer
tgggtataactggaactacggctggtt

SEQ ID NO: 90 - DNA, 26 bp, Artificial Sequence
  primer
cggatttaactggggatctcccctta

SEQ ID NO: 91 - DNA, 16 bp, Artificial Sequence
  primer
cccctccactcccccg

SEQ ID NO: 92 - DNA, 22 bp, Artificial Sequence
  primer
caaggtacacactggctgggaa

SEQ ID NO: 93 - DNA, 20 bp, Artificial Sequence
  primer
ctgccgacccactacatgga

SEQ ID NO: 94 - DNA, 31 bp, Artificial Sequence
  primer
tataacagctctacttctaccacacgaccta

SEQ ID NO: 95 - DNA, 20 bp, Artificial Sequence
  primer
ctacgtggaaccgtgaggct

SEQ ID NO: 96 - DNA, 19 bp, Artificial Sequence
  primer
cgtatcccccagtcgcaca

SEQ ID NO: 97 - DNA, 32 bp, Artificial Sequence
  primer
cggagggtaaattactatgatagtagtggttt

SEQ ID NO: 98 - DNA, 23 bp, Artificial Sequence
  primer
aaaagggtcttgggcgtttagga

SEQ ID NO: 99 - DNA, 17 bp, Artificial Sequence
  primer
gtccgtaccccttgccg

SEQ ID NO: 100 - DNA, 24 bp, Artificial Sequence
  primer
agttcctatccgagacctccaatt

SEQ ID NO: 101 - DNA, 20 bp, Artificial Sequence
  primer
aattcgggagtcgggggtat

SEQ ID NO: 102 - DNA, 27 bp, Artificial Sequence
  primer
agagaggagagcctagggatattttga

SEQ ID NO: 103 - DNA, 22 bp, Artificial Sequence
  primer
gcgagcaacaactggattttga

SEQ ID NO: 104 - DNA, 19 bp, Artificial Sequence
  primer
gaaccaacctccgaggcct

SEQ ID NO: 105 - DNA, 21 bp, Artificial Sequence
  primer
gttaatatggggccatctggg

SEQ ID NO: 106 - DNA, 19 bp, Artificial Sequence
  primer
agagggggctcccctatgg

SEQ ID NO: 107 - DNA, 19 bp, Artificial Sequence
  primer
agcagtggcatgccattga

SEQ ID NO: 108 - DNA, 17 bp, Artificial Sequence
  primer
cctgccccccgctacaa

SEQ ID NO: 109 - DNA, 23 bp, Artificial Sequence
  primer
caagctgagtctccctaagtgga

SEQ ID NO: 110 - DNA, 21 bp, Artificial Sequence
  primer
atatggcaaaaatgcagctgc

SEQ ID NO: 111 - DNA, 23 bp, Artificial Sequence
  primer
gcagaaaacaaaggccctagagt

SEQ ID NO: 112 - DNA, 23 bp, Artificial Sequence
  primer
caagctgagtctccctaagtgga

SEQ ID NO: 113 - DNA, 26 bp, Artificial Sequence
  primer
cagagttaaagcaggagagaggttgt

SEQ ID NO: 114 - DNA, 24 bp, Artificial Sequence
  primer
tgcaaagaacctggctgtacttaa

SEQ ID NO: 115 - DNA, 17 bp, Artificial Sequence
  primer
gctggaaggtggggaga

SEQ ID NO: 116 - DNA, 23 bp, Artificial Sequence
  primer
caagctgagtctccctaagtgga

SEQ ID NO: 117 - DNA, 23 bp, Artificial Sequence
  primer
gcagaaaacaaaggccctagagt

SEQ ID NO: 118 - DNA, 24 bp, Artificial Sequence
  primer
tgcaaagaacctggctgtacttaa

SEQ ID NO: 119 - DNA, 24 bp, Artificial Sequence
  primer
tgcaaagaacctggctgtacttaa

SEQ ID NO: 120 - DNA, 26 bp, Artificial Sequence
  primer
cagagttaaagcaggagagaggttgt

SEQ ID NO: 121 - DNA, 26 bp, Artificial Sequence
  primer
cagagttaaagcaggagagaggttgt

SEQ ID NO: 122 - DNA, 24 bp, Artificial Sequence
  primer
tgcaaagaacctggctgtacttaa

SEQ ID NO: 123 - DNA, 23 bp, Artificial Sequence
  primer
caagctgagtctccctaagtgga

SEQ ID NO: 124 - DNA, 26 bp, Artificial Sequence
  primer
cagagttaaagcaggagagaggttgt

SEQ ID NO: 125 - DNA, 24 bp, Artificial Sequence
  primer
tgcaaagaacctggctgtacttaa

SEQ ID NO: 126 - DNA, 21 bp, Artificial Sequence
  primer
atatggcaaaaatgcagctgc

SEQ ID NO: 127 - DNA, 23 bp, Artificial Sequence
  primer
gcagaaaacaaaggccctagagt

SEQ ID NO: 128 - DNA, 23 bp, Artificial Sequence
  primer
gcagaaaacaaaggccctagagt

SEQ ID NO: 129 - DNA, 20 bp, Artificial Sequence
  primer
tttgcccctgcagtttttgt

SEQ ID NO: 130 - DNA, 24 bp, Artificial Sequence
  primer
tgcaaagaacctggctgtacttaa

SEQ ID NO: 131 - DNA, 26 bp, Artificial Sequence
  primer
cagagttaaagcaggagagaggttgt

SEQ ID NO: 132 - DNA, 26 bp, Artificial Sequence
  primer
cagagttaaagcaggagagaggttgt

SEQ ID NO: 133 - DNA, 24 bp, Artificial Sequence
  primer
tgcaaagaacctggctgtacttaa

SEQ ID NO: 134 - DNA, 26 bp, Artificial Sequence
  primer
cagagttaaagcaggagagaggttgt

SEQ ID NO: 135 - DNA, 24 bp, Artificial Sequence
  primer
tgcaaagaacctggctgtacttaa

SEQ ID NO: 136 - DNA, 23 bp, Artificial Sequence
  primer
gcagaaaacaaaggccctagagt

SEQ ID NO: 137 - DNA, 26 bp, Artificial Sequence
  primer
cagagttaaagcaggagagaggttgt

SEQ ID NO: 138 - DNA, 18 bp, Artificial Sequence
  primer
ggcattccgtcaggcaaa

SEQ ID NO: 139 - DNA, 23 bp, Artificial Sequence
  primer
aggcagaaggaaagccatcttac

SEQ ID NO: 140 - DNA, 23 bp, Artificial Sequence
  primer
aggcagaaggaaagccatcttac

SEQ ID NO: 141 - DNA, 26 bp, Artificial Sequence
  primer
cagagttaaagcaggagagaggttgt

SEQ ID NO: 142 - DNA, 22 bp, Artificial Sequence
  primer
ctgcttgctgtgtttgtctcct

SEQ ID NO: 143 - DNA, 24 bp, Artificial Sequence
  probe
ccctggtcaccgtctcctcaggtg

SEQ ID NO: 144 - DNA, 24 bp, Artificial Sequence
  probe
agcccagggcgactcctcatgagt

SEQ ID NO: 145 - DNA, 27 bp, Artificial Sequence
  probe
cacggtcaccgtctcctcaggtaagaa

SEQ ID NO: 146 - DNA, 24 bp, Artificial Sequence
  probe
ccctggtcaccgtctcctcaggtg

SEQ ID NO: 147 - DNA, 24 bp, Artificial Sequence
  probe
ccctggtcaccgtctcctcaggtg

SEQ ID NO: 148 - DNA, 32 bp, Artificial Sequence
  probe
agacccttcatctctctctgatggtgcaagta

SEQ ID NO: 149 - DNA, 20 bp, Artificial Sequence
  probe
cgggcaccaggctcacggtc

SEQ ID NO: 150 - DNA, 24 bp, Artificial Sequence
  probe
ccctggtcaccgtctcctcaggtg

SEQ ID NO: 151 - DNA, 27 bp, Artificial Sequence
  probe
cacggtcaccgtctcctcaggtaagaa

SEQ ID NO: 152 - DNA, 32 bp, Artificial Sequence; identical to SEQ ID NO: 148
  probe

SEQ ID NO: 153 - DNA, 32 bp, Artificial Sequence; identical to SEQ ID NO: 148
  probe

SEQ ID NO: 154 - DNA, 24 bp, Artificial Sequence
  probe
ccctggtcaccgtctcctcaggtg

SEQ ID NO: 155 - DNA, 24 bp, Artificial Sequence
  probe
ccctggtcaccgtctcctcaggtg

SEQ ID NO: 156 - DNA, 32 bp, Artificial Sequence; identical to SEQ ID NO: 148
  probe

SEQ ID NO: 157 - DNA, 24 bp, Artificial Sequence
  probe
ccctggtcaccgtctcctcaggtg

SEQ ID NO: 158 - DNA, 24 bp, Artificial Sequence
  probe
ccctggtcaccgtctcctcaggtg

SEQ ID NO: 159 - DNA, 32 bp, Artificial Sequence; identical to SEQ ID NO: 148
  probe

SEQ ID NO: 160 - DNA, 24 bp, Artificial Sequence
  probe
agcccagggcgactcctcatgagt

SEQ ID NO: 161 - DNA, 27 bp, Artificial Sequence
  probe
cacggtcaccgtctcctcaggtaagaa

SEQ ID NO: 162 - DNA, 27 bp, Artificial Sequence
  probe
cacggtcaccgtctcctcaggtaagaa

SEQ ID NO: 163 - DNA, 31 bp, Artificial Sequence
  probe
cgcacagtgctacaaaacctacagagacctg

SEQ ID NO: 164 - DNA, 32 bp, Artificial Sequence; identical to SEQ ID NO: 148
  probe

SEQ ID NO: 165 - DNA, 24 bp, Artificial Sequence
  probe
ccctggtcaccgtctcctcaggtg

SEQ ID NO: 166 - DNA, 24 bp, Artificial Sequence
  probe
ccctggtcaccgtctcctcaggtg

SEQ ID NO: 167 - DNA, 32 bp, Artificial Sequence; identical to SEQ ID NO: 148
  probe

SEQ ID NO: 168 - DNA, 24 bp, Artificial Sequence
  probe
ccctggtcaccgtctcctcaggtg

SEQ ID NO: 169 - DNA, 32 bp, Artificial Sequence; identical to SEQ ID NO: 148
  probe

SEQ ID NO: 170 - DNA, 27 bp, Artificial Sequence
  probe
cacggtcaccgtctcctcaggtaagaa

SEQ ID NO: 171 - DNA, 24 bp, Artificial Sequence
  probe
ccctggtcaccgtctcctcaggtg

SEQ ID NO: 172 - DNA, 34 bp, Artificial Sequence
  probe
taggatacctgaaacgtctacatccactctcacc

SEQ ID NO: 173 - DNA, 27 bp, Artificial Sequence
  probe
caagggacaatggtcaccgtctcttca

SEQ ID NO: 174 - DNA, 27 bp, Artificial Sequence
  probe
caagggacaatggtcaccgtctcttca

SEQ ID NO: 175 - DNA, 24 bp, Artificial Sequence
  probe
ccctggtcaccgtctcctcaggtg

SEQ ID NO: 176 - DNA, 25 bp, Artificial Sequence
  probe
atatcctcaccctgggtcccatgcc

SEQ ID NO: 177 - DNA, 19 bp, Artificial Sequence
  iC9 forward primer
accagctggatgccatctc

SEQ ID NO: 178 - DNA, 20 bp, Artificial Sequence
  iC9 reverse primer
cagctgcctgactttggatc

SEQ ID NO: 179 - DNA, 23 bp, Artificial Sequence
  iC9 probe
  misc_feature (9)..(10): ZEN is inserted between nucleotide positions 9 and 10
agcctgcccacaccttctgacat

SEQ ID NO: 180 - DNA, 2817 bp, Artificial Sequence
  Nucleotide sequence encoding a polypetide comprising iC9 and anti-CD19 CAR
atgctcgagatgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaaagcggaggaggatccggagtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggccccgcgggaaggccgagggagcctgctgacatgtggcgatgtggaggaaaacccaggaccaccatggatggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggcggaggaagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 181 - protein, 9 aa, Artificial Sequence
  Immunogenic peptide
STNVSPAPR

SEQ ID NO: 182 - protein, 10 aa, Artificial Sequence
  Immunogenic peptide
GSELPTQGTF

SEQ ID NO: 183 - protein, 8 aa, Artificial Sequence
  Immunogenic peptide
ELPTQGTF

SEQ ID NO: 184 - protein, 10 aa, Artificial Sequence
  Immunogenic peptide
SVTVSSPAPR

SEQ ID NO: 185 - protein, 9 aa, Artificial Sequence
  Immunogenic peptide
VTVSSPAPR

SEQ ID NO: 186 - protein, 9 aa, Artificial Sequence
  linker
SGGGGSGGG

SEQ ID NO: 187 - protein, 11 aa, Artificial Sequence
  linker
SGGGGSGGGGS

SEQ ID NO: 188 - protein, 12 aa, Artificial Sequence
  linker
SGGGGSGGGGSG

SEQ ID NO: 189 - protein, 14 aa, Artificial Sequence
  linker
SGGGGSGGGGSGGG

SEQ ID NO: 190 - protein, 10 aa, Artificial Sequence
  linker
SGGGGSGGGG

SEQ ID NO: 191 - protein, 13 aa, Artificial Sequence
  linker
SGGGGSGGGGSGG